(19) United States
(12) Patent Application Publication
(10) Pub. No.: US 2024/0174714 A1
(43) Pub. Date: May 30, 2024

(54) TREATMENT OF SKIN DISORDERS

(71) Applicant: BACOBA AG, Buonas (CH)

(72) Inventors: Michael ADAMS, Uster (CH); Michael KEMMLER, Lörrach (DE); Rainer RIEDL, Wilen b. Wollerau (CH); Dimanthi PLIATSIKA, Wädenswil (CH); Georgios IMANIDIS, Bottmingen (CH); Susanne HERZIG, Grenzach-Wyhlen (DE); Marcel KAISER, Obermumpf (CH); Pascal MÄSER, Reinach (CH)

(21) Appl. No.: 18/264,332

(22) PCT Filed: Feb. 7, 2022
(86) PCT No.: PCT/EP2022/052891

§ 371 Date: Aug. 4, 2023

(30) Foreign Application Priority Data
Feb. 8, 2021 (EP) 21155878.8

(51) Int. Cl. C07K 7/02; A61K 9/00; A61P 31/04; A61P 31/10; A61P 33/02

(52) U.S. Cl. CPC C07K 7/02; A61K 9/0014; A61P 31/04; A61P 31/10; A61P 33/02

(57) ABSTRACT

The present invention relates to a compound of formula (I), pharmaceutically acceptable salts thereof as well as pharmaceutical compositions, for use in a method of topical treatment of a skin disease or skin disorder of a mammal, wherein said method comprises topical administration of said compound to said mammal, Formula (I) wherein R₁ is selected from carbocyclyl or heterocyclyl, each independently optionally substituted with C₁-C₄alkyl, halogen, oxo, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl; R₂ is selected from C₅-C₁₂alkyl, C₄-C₁₀alkoxy, C₁-C₃ alkylene-cycloalkyl, C₁-C₃alkylene-aryl, C₁-C₃alkylene-heteroaryl, wherein said alkyl, cycloalkyl, aryl and heteroaryl are each independently optionally substituted with one or more, typically and preferably one or two, substituents selected from C₁-C₂ alkyl, C₁-C₂ haloalkyl, halogen, C₁-C₂ alkoxy; R₃ is (Ia), wherein R₇, R₈, R₉ and R₁₀ are independently at each occurrence H or C₁-C₃alkyl, preferably H or methyl, or independently at each occurrence two of said R₇, R₈, R₉ and R₁₀ together with the carbon atom to which they are attached form a carbocyclic or heterocyclic ring, preferably a carbocyclic ring, and wherein R₁₁ and R₁₂ are independently of each other H or C₁-C₄ alkyl optionally substituted with halogen, hydroxyl or C₃-C₆cycloalkyl; or together with the nitrogen atom to which they are attached form independently at each occurrence a heteroaryl or a heterocyclyl, each independently optionally substituted with halogen, C₁-C₄alkyl, ORD, NR₁₄R₁₅; wherein R₁₃, R₁₄, R₁₅ are independently at each occurrence H, C₁-C₄alkyl, and wherein the arrow indicates the attachment to the C(O)-moiety depicted in formula (I).

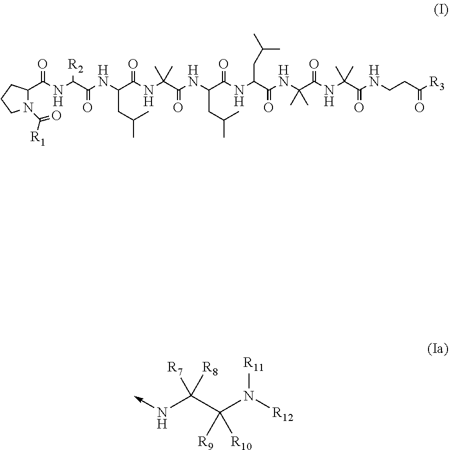

The present invention relates to compounds of formula (I), pharmaceutically acceptable salts thereof as well as pharmaceutical compositions comprising the same and, in particular, to the use of the compounds of formula (I) and pharmaceutical compositions in methods of topical treatment of skin diseases or skin disorders including protozoan skin diseases, bacterial skin infections and fungal skin infections.

### RELATED ART

The skin are recognized as an interesting route for drug delivery since topical administration of drugs for dermal and/or transdermal applications not only avoids the risks of toxicity and the gastrointestinal side-effects often associated with other classical treatments such as systemic treatments which may be parenteral or oral but further it has greater acceptability from patients and does not require of valuations before, during or after treatment to check toxicity levels of the liver, kidney and the like. Therefore, the development of drugs suitable for topical applications in particular application to the skin receives a great deal of interest.

The human skin is the largest and most accessible organ for drug delivery. However, it represents a natural physicochemical barrier of our bodies and as a result, it is characterized by low permeability, in order to limit the transport of most pathogens, toxins and drug molecules. Thus, drug delivery into and across the skin is a serious challenge.

The main transport barrier for drug delivery into or across the skin is the stratum corneum which is typically 10-20 μm thick and consists of 10-15 layers of corneocytes surrounded by a lipid-enriched lipid matrix composed of ceramides, cholesterol and free fatty acids. To penetrate through the stratum corneum drugs must navigate through the tortuous lipid pathways surrounding the keratin-rich cells, or repeatedly partition between the aqueous, keratin-rich phase and the lipid phase. In general, small molecules are able to penetrate the stratum corneum while, in contrast, the delivery of larger molecules, such as peptides and proteins, remains a major challenge. For pharmaceutical purposes, it has been argued to restrict the development of new compounds to a molecular weight (MW) of under 500 Dalton when topical dermatological therapy or percutaneous systemic therapy is addressed since around 500 Dalton is the start of a rapid decline in skin absorption due to molecular size. This is nowadays known as the “500 Dalton rule”. Further physicochemical properties of a topical drug beneficial to achieve passive transportation through the stratum corneum are a partition coefficient octanol/water logP between 1 and 3 and a water solubility greater than 1 mg/ml and the absence of polar centers (Bos and Meinardi, Exp Dermatol 2000, 9:165-169; Gorouhi and Maibach, International Journal of Cosmetic Science, 2009, 31, 327-345).

Skin diseases or skin disorders constitute a broad spectrum of diseases or disorders including inflammatory and infectious skin diseases or disorders. Infectious skin diseases or disorders can hereby be caused by bacteria, fungi, yeasts, viruses, or parasites.

Protozoan infections are parasitic diseases caused by organisms formerly classified in the Kingdom Protozoa. Protozoa are single-celled organism which can only be seen under a microscope. Protozoan infections include in particular leishmaniasis which is caused by parasites of the genus Leishmania. Leishmaniasis occurs in 3 main forms: cutaneous leishmaniasis, mucocutaneous leishmaniasis and visceral leishmaniasis (Akhoundi et al. Molecular Aspects of Medicine 2017, 57:1-29; Torres-Guerrero et al, F1000Research 2017, May 26; 6:750).

Cutaneous leishmaniasis (CL) is the most common form of leishmaniasis with estimated 1 million cases. The leishmaniases are caused by different species of the protozoan genus Leishmania, transmitted through the bite of hematophagous sand flies. Multiple Leishmania species produce CL in children and adults, primarily grouped in Old World cutaneous leishmaniasis (OWCL) species with L. major, L. tropica and L. aethiopica, L. infantum and L. chagasi; and New World cutaneous leishmaniasis (NWCL) species with L. mexicana, L. amazonenesis, L. braziliensis, L. panamensis, L. peruviana and L. guyanensis. CL produces disfiguring lesions on the infection sites, which are usually exposed body parts, like the face, arms and legs. There may be a large number of lesions, sometimes up to 200. Primary skin infections of CL sometimes resolve without treatment, with the host developing acquired immunity through cellular and humoral responses, but the infection can spread to produce secondary lesions in the skin (including diffuse cutaneous leishmaniasis), in the mucosa, disseminating to the upper oral and respiratory mucous membranes leading to mucocutaneous leishmaniasis and in the spleen, liver and bone marrow leading to visceral leishmaniasis, which is usually fatal if untreated. CL and MCL infections can lead to severe scarring and permanent disfigurement. Current drugs used to treat CL leave significant room for improvement related to efficacy, availability, safety, resistance, stability, low tolerability, long treatment duration and difficulty of administration. In the last WHO Research & Development Blueprint Review in 2018, the experts recognised the need for more effective therapeutics against leishmaniasis, and the WHO has called for new treatment options for localised CL, for which the efficacy of current medicines is limited, but for which, topical medicines and anti-infectives could be options.

Although many bacteria come in contact with or reside on the skin, they are normally unable to establish an infection. The skin provides a remarkably good barrier against bacterial infections. Bacterial skin infections develop when bacteria enter through hair follicles or through small breaks in the skin that result from scrapes, punctures, surgery, burns, sunburn, animal or insect bites, wounds, and pre-existing skin disorders. Many types of bacteria can infect the skin, while bacterial skin infections are usually caused by gram-positive strains of Staphylococcus and Streptococcus including the multi-resistant Staphylococcus aureus (MRSA) strains.

A fungal infection, also called mycosis, is a skin disease caused by a fungus. Mycoses are common and a variety of environmental and physiological conditions can contribute to the development of fungal diseases. Inhalation of fungal spores or localized colonization of the skin may initiate persistent infections; therefore, mycoses often start in the lungs or on the skin. Fungal infections of the skin was the 4th most common skin disease in 2010 affecting 984 million people. An estimated 1.6 million people die each year of fungal infections. Dermatophytes are a group of closely related keratinophilic fungi that can invade keratinized human and animal tissues such as skin, hair and nails causing dermatophytosis. They are an important cause of superficial fungal infection. Trichophyton is a genus of fungi, it belongs to dermatophytes and includes the parasitic varieties that cause tinea, including athlete's foot, ringworm, jock itch, and similar infections of the nail, beard, skin and scalp. Candida is a genus of yeasts and is the most common cause of fungal infections worldwide. Candida is located on most of mucosal surfaces and mainly the gastrointestinal tract, along with the skin. Candida albicans is the most commonly isolated species and can cause infections (candidiasis or thrush) in humans and other animals.

EP 3345917 disclosed a new class of synthetic leucinostatin derivatives and its use for the treatment of protozoan infections.

### SUMMARY OF THE INVENTION

We have surprisingly found that the inventive compounds are suitable for topical application and administration as potential pharmaceutical products for the topical treatment of skin diseases or skin disorders. In particular, it has not only been found that the inventive compounds are able to penetrate into skin as shown by pig skin permeating experiments but are active against protozoan diseases, bacterial and fungal infections as shown by in vitro assays. Moreover, a preferred inventive compound shows successful treatment and anti-leishmanial effects in an in vivo mouse model of cutaneous leishmaniasis (CL) using C67BLC/6 mice infected with Leishmania mexicana where the topical drug treatment led to the reduction of the parasite loads in the treated group of mice by about 3 orders of magnitude as compared to the control. Furthermore, in an in vivo dermal infection mouse model using strains of Staphylococcus aureus (USA300 MRSA, BAA-1556, CFU/skin) a 71% reduction in colony forming units (CFUs) over the control group has been achieved.

Thus, in a first aspect, the present invention provides a compound of formula (I) for use in a method of topical treatment of a skin disease or skin disorder of a mammal, wherein said method comprises topical administration of said compound to said mammal, preferably to a human,

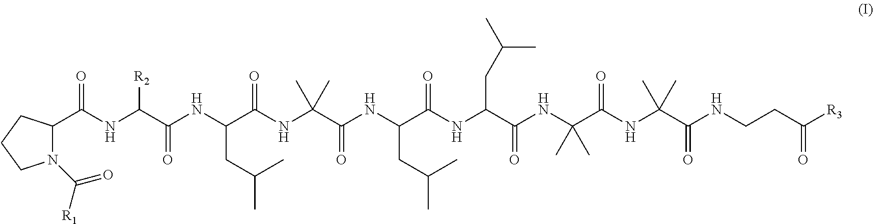

wherein
R₁ is selected from carbocyclyl or heterocyclyl, each independently optionally substituted with C₁-C₄alkyl, halogen, oxo, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl;
R₂ is selected from C₅-C₁₂alkyl, C₄-C₁₀alkoxy, C₁-C₃alkylene-cycloalkyl, C₁-C₃alkylene-aryl, C₁-C₃alkylene-heteroaryl, wherein said alkyl, cycloalkyl, aryl and heteroaryl are each independently optionally substituted with one or more, typically and preferably, one or two substituents selected from C₁-C₂alkyl, C₁-C₂haloalkyl, halogen, C₁-C₂alkoxy;
R₃ is

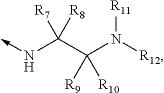

wherein
   R₇, R₈, R₉ and R₁₀ are independently at each occurrence H or C₁-C₃alkyl, preferably H or methyl, or independently at each occurrence two of said R₇, R₈, R₉ and R₁₀ together with the carbon atom to which they are attached form a carbocyclic or heterocyclic ring, preferably a carbocyclic ring, and wherein
      R₁₁ and R₁₂ are independently of each other H or C₁-C₄alkyl optionally substituted with halogen, hydroxyl or C₃-C₆cycloalkyl; or together with the nitrogen atom to which they are attached form independently at each occurrence a heteroaryl or a heterocyclyl, each independently optionally substituted with halogen, C₁-C₄alkyl, OR₁₃, NR₁₄R₁₅; wherein R₁₃, R₁₄, R₁₅ are independently at each occurrence H, C₁-C₄alkyl, and wherein the arrow indicates the attachment to the C(O)-moiety depicted in formula (I);
         and pharmaceutically acceptable salts of said compound of formula (I), wherein preferably said method comprises topical administration of an effective amount of said compound to said mammal, preferably to a human.

In a further aspect, the present invention provides a compound of formula (I) for use in a method of treating a skin disease or skin disorder of a mammal, wherein said method comprises topical administration of said compound to said mammal, preferably to a human,

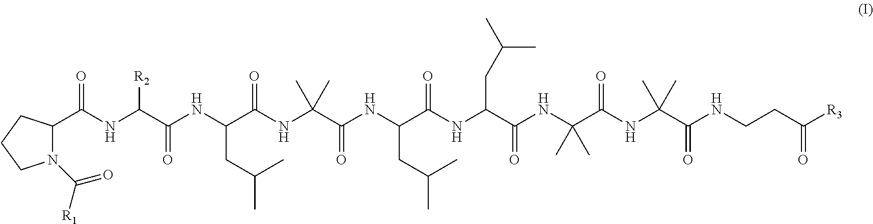

wherein
R₁ is selected from carbocyclyl or heterocyclyl, each independently optionally substituted with C₁-C₄alkyl, halogen, oxo, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl;
R₂ is selected from C₅-C₁₂alkyl, C₄-C₁₀alkoxy, C₁-C₃alkylene-cycloalkyl, C₁-C₃alkylene-aryl, C₁-C₃alkylene-heteroaryl, wherein said alkyl, cycloalkyl, aryl and heteroaryl are each independently optionally substituted with one or more, typically and preferably, one or two substituents selected from C₁-C₂alkyl, C₁-C₂haloalkyl, halogen, C₁-C₂alkoxy;
R₃ is

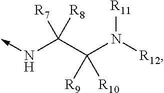

wherein
   R₇, R₈, R₉ and R₁₀ are independently at each occurrence H or C₁-C₃alkyl, preferably H or methyl, or independently at each occurrence two of said R₇, R₈, R₉ and R₁₀ together with the carbon atom to which they are attached form a carbocyclic or heterocyclic ring, preferably a carbocyclic ring, and wherein
      R₁₁ and R₁₂ are independently of each other H or C₁-C₄alkyl optionally substituted with halogen, hydroxyl or C₃-C₆cycloalkyl; or together with the nitrogen atom to which they are attached form independently at each occurrence a heteroaryl or a heterocyclyl, each independently optionally substituted with halogen, C₁-C₄alkyl, OR₁₃, NR₁₄R₁₅, wherein R₁₃, R₁₄, R₁₅ are independently at each occurrence H, C₁-C₄alkyl, and wherein the arrow indicates the attachment to the C(O)-moiety depicted in formula (I);
         and pharmaceutically acceptable salts of said compound of formula (I), wherein preferably said method comprises topical administration of an effective amount of said compound to said mammal, preferably to a human.

In a further aspect, the present invention provides a pharmaceutical composition for use in a method of topical treatment of a skin disease or skin disorder of a mammal, wherein said pharmaceutical composition comprises a compound of formula (I) or a pharmaceutically acceptable salt thereof as defined herein, in particular as in any one of the claims 1 to 14, and a pharmaceutically acceptable carrier or adjuvant, and wherein said method comprises topical administration of said pharmaceutical composition to said mammal, wherein preferably said pharmaceutical composition an effective amount of said compound.

Thus, in particular, the present invention provides for compounds and pharmaceutical compositions for use in methods of topical treatment of skin diseases or disorders, in particular skin infections, which skin diseases or disorders, in particular skin infections, are caused by protozoal parasites, and here in particular by species of the genus Leishmania, by bacteria, and here in particular by gram positive bacteria such as those of the genera Staphylococcus or Streptococcus including multi-resistant Staphylococcus aureus (MRSA) strains, which show resistance to known antibiotics like Methicillin, Oxacillin, Gentamicin, Levofloxacin, Vancomycin, Clindamycin, Erythromycin, and/or Linezolid, as well by fungi, and here in particular by fungi of the genus Trichophyton, wherein said methods comprise topical administration of an effective amount of a compound of formula (I) to a mammal, preferably to a human.

In another aspect, the present invention provides a method for topically treating a skin disease or skin disorder of a mammal, preferably of a human, wherein said method comprises topical administration of a compound of formula (I) of the present invention, or the pharmaceutical composition comprising a compound of formula (I), to said mammal, preferably to said human, and wherein preferably said method comprises topical administration of an effective amount of said compound to said mammal, preferably to said human.

In a further aspect, the present invention provides for the use of a compound of formula (I) of the present invention, or the pharmaceutical composition comprising a compound of formula (I), for the preparation of a medicament for topically treating a skin disease or skin disorder of a mammal, preferably of a human, wherein said method comprises topical administration of said compound of formula (I), or said pharmaceutical composition of the invention, to said mammal, preferably to said human, and wherein preferably said method comprises topical administration of an effective amount of said compound, or said pharmaceutical composition, to said mammal, preferably to said human.

Further aspects and embodiments of the present invention will be become apparent as this description continues.

### DESCRIPTION OF FIGS

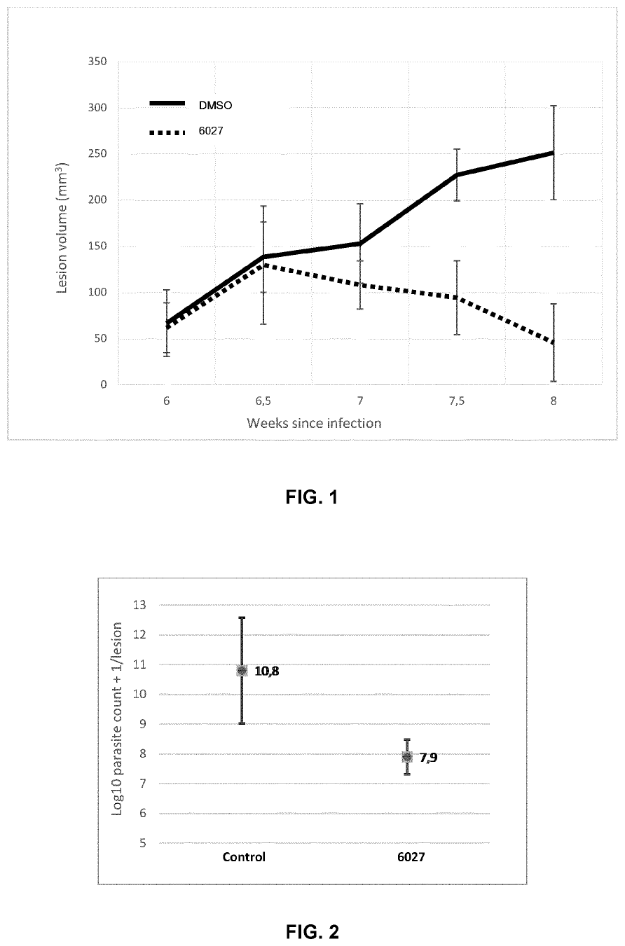

FIG. 1: shows the cutaneous leishmaniasis (CL) lesion progression in Leishmania. mexicana infected C₆₇BLC/6 mice. Measurements were done every 3 days by calliper over the 14-day treatment duration and shown in mm 3 with standard deviation (SD). The volume of the induced CL lesions gradually reduced in the 6027 treated group over time, whereas lesions in the vehicle (DMSO) control group increased in volume.

FIG. 2: shows parasite load in lesions in L. mexicana infected C₆₇BLC/6 mice after 14-day treatment. Quantification of parasites in mouse lesions treated with 6027 and vehicle control (DMSO) for 14 days (N=6 each per group). Parasites were harvested from lesions and cultivated for 10 days. Values represent the highest log dilution with viable parasites, shown with standard deviation (SD).

### DETAILED DESCRIPTION OF THE INVENTION

Unless defined otherwise, all technical and scientific terms used herein have the same meanings as commonly understood by one of ordinary skill in the art to which this invention belongs. The herein described and disclosed embodiments, preferred embodiments and very preferred embodiments should apply to all aspects and other embodiments, preferred embodiments and very preferred embodiments irrespective of whether is specifically again referred to.

When the terms “a,” or “an” are used herein, they mean “at least one” unless indicated otherwise.

Each alkyl moiety either alone or as part of a larger group such as alkoxy, aminoalkyl or haloalkoxy or alkylene refers to a monovalent saturated acyclic (i.e., non-cyclic) hydrocarbon group which may be linear or branched. Accordingly, an “alkyl” group does not comprise any carbon-to-carbon double bond or any carbon-to-carbon triple bond. A “C1-6 alkyl” denotes an alkyl group having 1 to 6 carbon atoms. Examples include methyl, ethyl, n-propyl, prop-2-yl, n-butyl, but-2-yl, 2-methyl-prop-1-yl or 2-methyl-prop-2-yl. Examples of an alkoxy include methoxy, ethoxy, propoxy, iso-propoxy, n-butoxy, sec-butoxy, tert-butoxy, n-pentoxy, neo-pentoxy, n-hexoxy. Examples of aminoalkyl include aminomethyl, aminoethyl, dimethylaminomethyl, dimethylaminoethyl. Haloalkoxy refers to alkoxy with further substitution of halogen.

As used herein, the term “alkylene” refers to an alkanediyl group, i.e. a divalent saturated acyclic hydrocarbon group which may be linear or branched. A “C₁₋₆alkylene” denotes an alkylene group having 1 to 6 carbon atoms, and the term “C₀₋₃alkylene” indicates that a covalent bond (corresponding to the option “C₀alkylene”) or a C₁₋₃alkylene is present. Preferred exemplary alkylene groups are methylene (—CH₂—), ethylene (e.g., —CH₂—CH₂— or —CH(—CH₃)—), propylene (e.g., —CH₂—CH₂—CH₂—, —CH(—CH₂—CH₃)—, —CH₂—CH(—CH₃)—, or —CH(—CH₃)—CH₂—), or butylene (e.g., —CH₂—CH₂—CH₂—CH₂—).

Each haloalkyl moiety either alone or as part of a larger group such as haloalkoxy is an alkyl group substituted by one or more of the same or different halogen atoms. Haloalkyl include for example 1 to 5 halo substituents, or 1 to 3 halo substituents. Examples include in particular fluoromethyl, difluoromethyl, trifluoromethyl, chlorodifluoromethyl and 2,2,2-trifluoro-ethyl.

Each alkenyl moiety either alone or as part of a larger group such as alkenyloxy or alkenylene is a straight or branched chain and is preferably C₂-C₁₆alkenyl, more preferably C₂-C₁₄alkenyl. Each moiety can be of either the (E)- or (Z)-configuration. Examples include vinyl and allyl. A compound of the present invention comprising an alkenyl moiety thus may include, if applicable, either said compound with said alkenyl moiety in its (E)-configuration, said compound with said alkenyl moiety in its (Z)-configuration and mixtures thereof in any ratio.

Halogen is fluorine, chlorine, bromine, or iodine.

As used herein, the term “carbocyclyl” refers to a monovalent hydrocarbon ring group, including monocyclic rings as well as bridged ring, spiro ring and/or fused ring systems (which may be composed, e.g., of two or three rings), wherein said ring group may be saturated, partially unsaturated (i.e., unsaturated but not aromatic) or aromatic. Unless defined otherwise, “carbocyclyl” preferably refers to aryl, cycloalkyl or cycloalkenyl. The number of carbon atoms in the carbocyclyl group is not particularly limited and is preferably 3 to 14, more preferably 4 to 12 or 5 to 10.

As used herein, the term “heterocyclyl” refers to a monovalent ring group, including monocyclic rings as well as bridged ring, spiro ring and/or fused ring systems (which may be composed, e.g., of two or three rings), wherein said ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group may be saturated, partially unsaturated (i.e., unsaturated but not aromatic) or aromatic. Unless defined otherwise, “heterocyclyl” preferably refers to heteroaryl, heterocycloalkyl or heterocycloalkenyl. The number of carbon atoms in the heterocyclyl group is not particularly limited and is preferably 5 to 14, more preferably 5 to 12 or 5 to 10.

The term “aryl”, as used herein, refers to a monovalent aromatic hydrocarbon radical of 6-14 carbon atoms (C₆-C₁₄). Aryl includes bicyclic, tricyclic or tetracyclic, preferably bicyclic, radicals comprising an aromatic ring to which saturated, partially unsaturated or aromatic carbocyclic or heterocyclic ring(s) are fused or bridged. Aryl groups are optionally substituted independently with one or more substituents, typically and preferably with one or two substituents, wherein said substituents are typically and preferably independently at each occurrence selected from C₁-C₄alkyl, halogen, oxo, CF₃, OH, OC₁-C₃alkyl, NH₂, NH(C₁-C₃alkyl), N(C₁-C₃alkyl)₂, C₆H₅, C₆H₅ substituted with halogen, C₁-C₃alkyl, OH, OC₁-C₃alkyl, NH₂, NH(C₁-C₃alkyl), N(C₁-C₃alkyl)₂. Typical aryl groups include, but are not limited to, phenyl, substituted phenyls, naphthyl, 1,2-dihydronapthalenyl, 1,2,3,4-tetrahydronaphthenyl, anthracenyl, phenanthrenyl, biphenyl, indenyl and indanyl. Unless defined otherwise, an “aryl” preferably has 5 to 14 ring atoms, more preferably 5 to 10 ring atoms, and most preferably refers to phenyl or phenyl substituted by one or two substituents, preferably by one substituent, independently selected from C₁-C₄alkyl, halogen, CF₃, OH, OC₁-C₃alkyl, NH₂, NH(C₁-C₃alkyl), N(C₁-C₃alkyl)₂, C₆H₅, C₆H₅ substituted with halogen, C₁-C₃alkyl, OH, OC₁-C₃alkyl, NH₂, NH(C₁-C₃alkyl), N(C₁-C₃alkyl)₂.

As used herein, the term “heteroaryl” refers to an aromatic ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic), wherein said aromatic ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). Heteroaryl groups are optionally substituted independently with one or more substituents, typically and preferably with one or two substituents independently selected from C₁-C₄alkyl, halogen, oxo, CF₃, OH, OC₁-C₃alkyl, NH₂, NH(C₁-C₃alkyl), N(C₁-C₃alkyl)₂, C₆H₅, C₆H₅ substituted with halogen, C₁-C₃alkyl, OH, OC₁-C₃alkyl, NH₂, NH(C₁-C₃alkyl), N(C₁-C₃alkyl)₂. Unless defined otherwise, a “heteroaryl” preferably has 5 to 14 ring atoms, more preferably 5 to 12 or 5 to 10 ring atoms.

In a preferred embodiment, said heteroaryl is a monovalent monocyclic aromatic or bicyclic aromatic ring group, wherein said aromatic ring group comprises one or more (such as, e.g., one, two, three, or four) ring heteroatoms independently selected from O, S and N, and the remaining ring atoms are carbon atoms, and wherein said aromatic ring group is optionally substituted independently with one or more substituents, typically and preferably with one or two substituents independently selected from C₁-C₄alkyl, halogen, oxo, CF₃, OH, OC₁-C₃alkyl, NH₂, NH(C₁-C₃alkyl), N(C₁-C₃alkyl)₂, C₆H₅, C₆H₅ substituted with halogen, C₁-C₃alkyl, OH, OC₁-C₃alkyl, NH₂, NH(C₁-C₃alkyl), N(C₁-C₃alkyl)₂. Unless defined otherwise, such a monocyclic or bicyclic heteroaryl preferably has 5 to 12, preferably 5 to 10 ring atoms.

Examples of heteroaryl groups are pyridinyl, imidazolyl, imidazopyridinyl, pyrimidinyl, pyrazolyl, triazolyl, pyrazinyl, tetrazolyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, thiadiazolyl, furazanyl, benzofurazanyl, benzopyranyl, benzothiophenyl, benzothiazolyl, benzooxazolyl, quinazolinyl, quinoxalinyl, naphthyridinyl, and furopyridinyl, wherein said heteroaryl are optionally substituted independently with one or more, preferably one or two substituents independently selected from C₁-C₄alkyl, halogen, oxo, CF₃, OH, OC₁-C₃alkyl, NH₂, NH(C₁-C₃alkyl), N(C₁-C₃alkyl)₂, C₆H₅, C₆H₅ substituted with halogen, C₁-C₃alkyl, OH, OC₁-C₃alkyl, NH₂, NH(C₁-C₃alkyl), N(C₁-C₃alkyl)₂.

Further examples of such monocyclic heteroaryl radicals include, but are not limited to: 2-pyridyl, 3-pyridyl, 4-pyridyl, 3-isoxazolyl, 4-isoxazolyl, 5-isoxazolyl, 2-imidazolyl, 4-imidazolyl, 3-pyrazolyl, 4-pyrazolyl, 2-pyrrolyl, 3-pyrrolyl, 2-thiazolyl, 4-thiazolyl, 5-thiazolyl, 3-pyridazinyl, 4-pyridazinyl, 5-pyridazinyl, 2-pyrimidinyl, 5-pyrimidinyl, 6-pyrimidinyl, 2-pyrazinyl, 2-oxazolyl, 4-oxazolyl, 5-oxazolyl, 2-furanyl, 3-furanyl, 2-thienyl, 3-thienyl, 3-triazolyl, 1-triazolyl, 5-tetrazolyl, 1-tetrazolyl, and 2-tetrazolyl, which are optionally substituted independently with one or more substituents, typically and preferably with one or two substituents, wherein said substituents are independently at each occurrence independently selected from C₁-C₄alkyl, halogen, oxo, CF 3, OH, OC₁-C₃alkyl, NH₂, NH(C₁-C₃alkyl), N(C₁-C₃alkyl)₂, C₆H₅, C₆H₅ substituted with halogen, C₁-C₃alkyl, OH, OC₁-C₃alkyl, NH₂, NH(C₁-C₃alkyl), N(C₁-C₃alkyl)₂.

As used herein, the term “cycloalkyl” refers to a monovalent saturated hydrocarbon ring group, including monocyclic rings as well as bridged ring, spiro ring and/or fused ring systems (which may be composed, e.g., of two, three or four rings; such as, e.g., a fused ring system composed of two or three fused rings). Cycloalkyl groups are optionally substituted independently with one or more substituents, typically and preferably with one or two substituents independently selected from C₁-C₄alkyl, halogen, oxo, CF 3, OH, OC₁-C₃alkyl, NH₂, NH(C₁-C₃alkyl), N(C₁-C₃alkyl)₂, C₆H₅, C₆H₅ substituted with halogen, C₁-C₃alkyl, OH, OC₁-C₃alkyl, NH₂, NH(C₁-C₃alkyl), N(C₁-C₃alkyl)₂. Unless defined otherwise, “cycloalkyl” preferably refers to a C₃₋₁₄ cycloalkyl. Examples of cycloalkyl are cyclopropyl, cyclobutyl, cyclopentyl, cyclohexyl, cycloheptyl, or adamantyl.

As used herein, the term “heterocycloalkyl” refers to a monovalent saturated ring group, including monocyclic rings as well as bridged ring, Spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). Heterocycloalkyl groups are optionally substituted independently with one or more substituents, typically and preferably with one or two substituents independently selected from C₁-C₄alkyl, halogen, oxo, CF₃, OH, OC₁-C₃alkyl, NH₂, NH(C₁-C₃alkyl), N(C₁-C₃alkyl)₂, C₆H₅, C₆H₅ substituted with halogen, C₁-C₃alkyl, OH, OC₁-C₃alkyl, NH₂, NH(C₁-C₃alkyl), N(C₁-C₃alkyl)₂. Unless defined otherwise, “heterocycloalkyl” preferably refers to a 3 to 14 membered saturated ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, and wherein one or more carbon ring atoms are optionally oxidized; more preferably, “heterocycloalkyl” refers to a 5 to 7 membered saturated monocyclic ring group containing one or more (e.g., one, two, or three) ring heteroatoms independently selected from O, S and N, wherein one or more S ring atoms (if present) and/or one or more N ring atoms (if present) are optionally oxidized, and wherein one or more carbon ring atoms are optionally oxidized.

As used herein, the term “cycloalkenyl” refers to an unsaturated alicyclic (non-aromatic) hydrocarbon ring group, including monocyclic rings as well as bridged ring, Spiro ring and/or fused ring systems (which may be composed, e.g., of two or three rings; such as, e.g., a fused ring system composed of two or three fused rings), wherein said hydrocarbon ring group comprises one or more (e.g., one or two) carbon-to-carbon double bonds and does not comprise any carbon-to-carbon triple bond. Cycloalkenyl groups are optionally substituted independently with one or more substituents, typically and preferably with one or two substituents, wherein said substituents are typically and preferably independently at each occurrence selected from C₁-C₄alkyl, halogen, oxo, CF₃, OH, OC₁-C₃alkyl, NH₂, NH(C₁-C₃alkyl), N(C₁-C₃alkyl)₂, C₆H₅, C₆H₅ substituted with halogen, C₁-C₃alkyl, OH, OC₁-C₃alkyl, NH₂, NH(C₁-C₃alkyl), N(C₁-C₃alkyl)₂. “Cycloalkenyl” may, e.g., refer to cyclopropenyl, cyclobutenyl, cyclopentenyl, cyclohexenyl, cyclohexadienyl, cycloheptenyl, or cycloheptadienyl. Unless defined otherwise, “cycloalkenyl” preferably refers to a C₃₋₁₄cycloalkenyl, and more preferably refers to a C₃₋₇ cycloalkenyl.

As used herein, the term “heterocycloalkenyl” refers to an unsaturated alicyclic (non-aromatic) ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms and carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group comprises at least one double bond between adjacent ring atoms and does not comprise any triple bond between adjacent ring atoms. Heterocycloalkenyl groups are optionally substituted independently with one or more substituents, typically and preferably with one or two substituents, wherein said substituents are typically and preferably independently at each occurrence selected from C₁-C₄alkyl, halogen, oxo, CF₃, OH, OC₁-C₃alkyl, NH₂, NH(C₁-C₃alkyl), N(C₁-C₃alkyl)₂, C₆H₅, C₆H₅ substituted with halogen, C₁-C₃alkyl, OH, OC₁-C₃alkyl, NH₂, NH(C₁-C₃alkyl), N(C₁-C₃alkyl)₂. Unless defined otherwise, “heterocycloalkenyl” preferably refers to a 3 to 14 membered unsaturated alicyclic ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms.

The terms “spiranyl”, and “spirocycle” are used interchangeably herein and refer to a saturated or a partially unsaturated (i.e., having one or more double within the rings), typically and preferably to a saturated, bicyclic radical of 3 to 8 ring atoms per ring, wherein the rings are linked together by one common atom, typically and preferably by a carbon atom. The individual rings may be both carbocyclic rings or may contain independently of each other at least one heteroatom selected from nitrogen, oxygen and sulphur, the remaining ring atoms being C, where one or both ring atoms are optionally substituted independently with one or more substituents as described herein. Examples of heterocyclic spiranyl include diazaspiranyl of the formulas —([CH₂]ₘ)ₙC([CH₂]ₘ)ₙ—N—C₁-C₃alkyl wherein m and n are independently at each occurrence selected from 1 or 2, typically and preferably —([CH₂])₂C([CH₂])₂—N—C₁-C₃alkyl, —([CH₂])₂C([CH_{2]2})—N—C₁-C₃alkyl, —([CH₂]₂)₂C([CH₂]₂)₂—N—C₁-C₃alkyl, ([CH₂]₂[CH₂])C([CH₂]₂[CH₂])—N—C₁-C₃alkyl.

Where a group is said to be optionally substituted, preferably there are optionally 1-5 substituents, more preferably optionally 1-3 substituents, and again more preferably optionally one or two substituents.

The term “amino acid”, as used herein, refers to organic compounds containing the functional groups amine (—NH₂) and carboxylic acid (—COOH) and its zwitterions, typically and preferably, along with a side chain specific to each amino acid. The term “amino acid” typically and preferably includes amino acids that occur naturally, such as proteinogenic amino acids (produced by RNA-translation), non-proteinogenic amino acids (produced by other metabolic mechanisms, e.g. posttranslational modification), standard or canonical amino acids (that are directly encoded by the codons of the genetic code) and non-standard or non-canonical amino acids (not directly encoded by the genetic code). Naturally occurring amino acids include non-eukaryotic and eukaryotic amino acids. The term “amino acid”, as used herein, also includes unnatural amino acids that are chemically synthesized. Moreover, the term covers alpha- (α-), beta- (β-), gamma- (γ-) and delta- (δ-) etc. amino acids as well as mixtures thereof in any ratio, and any isomeric form of an amino acid, i.e. D- and L-stereoisomers (alternatively addressed by the (R) and (S) nomenclature) as well as mixtures thereof in any ratio, preferably in a racemic ratio of 1:1. Amino acids in this invention are preferably in L-configuration. The term “D-stereoisomer”, “L-stereoisomer”, “D-amino acid” or “L-amino acid” refers to the chiral alpha carbon of the amino acids.

Certain compounds of formula (I) of the present invention may contain one or two or more centers of chirality and such compounds may be provided as pure enantiomers or pure diastereoisomers as well as mixtures thereof in any ratio. The compounds of the invention also include all tautomeric forms of the compounds of formula (I). The compounds of formula (I) may also be solvated, especially hydrated, which are also included in the compounds of formula (I). The term “chiral” refers to compounds, which have the property of non-superimposability of the mirror image partner, while the term “achiral” refers to compounds, which are superimposable on their mirror image partner. The term “stereoisomers” refers to compounds, which have identical chemical constitution, but differ with regard to the arrangement of the atoms or groups in space. “Diastereomer” refers to a stereoisomer with two or more centers of chirality in which the compounds are not mirror images of one another. Diastereomers have different physical properties, e.g. melting points, boiling points, spectral properties, and chemical and biological reactivities. Mixtures of diastereomers may be separated under high resolution analytical procedures such as electrophoresis and chromatography. “Enantiomers” refer to two stereoisomers of a compound which are non-superimposable mirror images of one another. Stereochemical definitions and conventions used herein generally follow S. P. Parker, Ed., McRaw-Hiff Dictionary of Chemical Terms (1984), McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric or a scalemic mixture. A 50:50 mixture of enantiomers is referred to as a racemic mixture or a racemate. The term “tautomer” or “tautomeric form” refers to structural isomers of different energies, which are interconvertible via a low energy barrier. For example, proton tautomers include interconversions via migration of a proton, such as keto-enol and imine-enamine isomerizations.

The phrase “pharmaceutically acceptable salt” as used herein, refers to pharmaceutically acceptable organic or inorganic salts of a compound of the invention, in particular acid addition salts. Exemplary salts include, but are not limited to, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate (mesylate), ethanesulfonate, benzenesulfonate, p-toluenesulfonate, and pamoate salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion. If the compound of the invention is a base, the desired pharmaceutically acceptable salt may be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, trifluoroacetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

A “solvate” refers to an association or complex of one or more solvent molecules and a compound of the invention. Examples of solvents that form solvates include, but are not limited to, water, isopropanol, ethanol, methanol, dimethyl sulfoxide (DMSO), ethyl acetate, acetic acid, and ethanolamine. The term “hydrate” refers to the complex where the solvent molecule is water.

The terms “compound of this invention” and “compounds of the present invention” and “compounds of formula (I)” include stereoisomers, geometric isomers, tautomers, solvates, pharmaceutically acceptable salts, and solvates of the salts thereof.

The term “mammal” includes, but is not limited to, humans, mice, rats, guinea pigs, monkeys, dogs, cats, horses, cows, pigs, and sheep. The term “mammal”, as used herein, preferably refers to humans.

The term “treatment” of a disorder or disease as used herein (e.g., “treatment” of cutaneous leishmaniasis) is well known in the art. “Treatment” of a disorder or disease implies that a disorder or disease is suspected or has been diagnosed in a patient/subject. A patient/subject suspected of suffering from a disorder or disease typically shows specific clinical and/or pathological symptoms which a skilled person can easily attribute to a specific pathological condition (i.e., diagnose a disorder or disease).

The “treatment” of a disorder or disease may, for example, lead to a halt in the progression of the disorder or disease (e.g., no deterioration of symptoms) or a delay in the progression of the disorder or disease (in case the halt in progression is of a transient nature only). The “treatment” of a disorder or disease may also lead to a partial response (e.g., amelioration of symptoms) or complete response (e.g., disappearance of symptoms) of the subject/patient suffering from the disorder or disease. Accordingly, the “treatment” of a disorder or disease may also refer to an amelioration of the disorder or disease, which may, e.g., lead to a halt in the progression of the disorder or disease or a delay in the progression of the disorder or disease. Such a partial or complete response may be followed by a relapse. It is to be understood that a subject/patient may experience a broad range of responses to a treatment. The treatment of a disorder or disease may, inter alia, comprise curative treatment (preferably leading to a complete response and eventually to healing of the disorder or disease) and palliative treatment (including symptomatic relief). The “amelioration” of a disorder or disease may, for example, lead to a halt in the progression of the disorder or disease or a delay in the progression of the disorder or disease. “Treatment” can also mean prolonging survival as compared to expected survival if not receiving treatment.

The term “effective amount” means an amount of a compound of the present invention that (i) treats the particular disease or disorder or (ii) attenuates, ameliorates, or eliminates one or more symptoms of the particular disease or disorder. In the case of protozoan diseases, in particular in case of cutaneous leishmaniasis, the effective amount of the drug may reduce the number of Leishmania parasites and/or reduce clinical symptoms.

The term “topical administration” as used herein, refers to application to body surfaces such as the skin. Typically and preferably, the term “topical administration” as used herein, refers to epicutaneous application meaning application directly to the skin. Thus, in a very preferred embodiment of the present invention, said topical administration is application on the skin, wherein preferably said topical administration is application directly on the skin. Typically and preferably, the present invention relates to locally applied and locally acting compounds for cutaneous use to cure infections of the skin, as referred to in the quality-equivalence guidelines for topical products of EMA (https://www.ema.europa.eu/en/documents/scientific-guideline/draft-guideline-quality-equivalence-topical-products_en.pdf).

In a first aspect, the present invention provides for a compound of formula (I) for use in a method of topical treatment of a skin disease or skin disorder of a mammal, wherein said method comprises topical administration of an effective amount of said compound to said mammal,

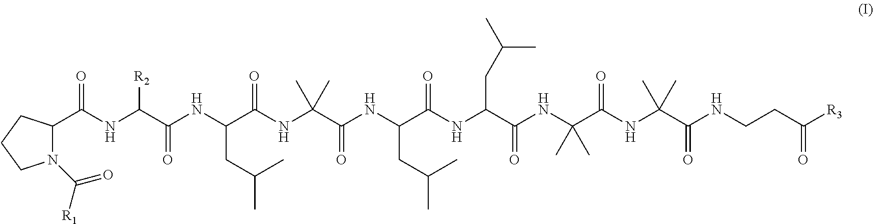

wherein
R₁ is selected from carbocyclyl or heterocyclyl, each independently optionally substituted with C₁-C₄alkyl, halogen, oxo, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl;
R₂ is selected from C₅-C₁₂alkyl, C₄-C₁₀alkoxy, C₁-C₃alkylene-cycloalkyl, C₁-C₃alkylene-aryl, C₁-C₃alkylene-heteroaryl, wherein said alkyl, cycloalkyl, aryl and heteroaryl are each independently optionally substituted with one or more, typically and preferably, one or two substituents selected from C₁-C₂alkyl, C₁-C₂haloalkyl, halogen,
C₁-C₂alkoxy;
R₃ is

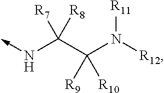

wherein
   R₇, R₈, R₉ and R₁₀ are independently at each occurrence H or C₁-C₃alkyl, preferably H or methyl, or independently at each occurrence two of said R₇, R₈, R₉ and R₁₀ together with the carbon atom to which they are attached form a carbocyclic or heterocyclic ring, preferably a carbocyclic ring, and wherein
      R₁₁ and R₁₂ are independently of each other H or C₁-C₄alkyl optionally substituted with halogen, hydroxyl or C₃-C₆cycloalkyl; or together with the nitrogen atom to which they are attached form independently at each occurrence a heteroaryl or a heterocyclyl, each independently optionally substituted with halogen, C₁-C₄alkyl, OR₁₃, NR₁₄R₁₅; wherein R₁₃, R₁₄, R₁₅ are independently at each occurrence H, C₁-C₄alkyl, and wherein the arrow indicates the attachment to the C(O)-moiety depicted in formula (I);
and pharmaceutically acceptable salts of said compound of formula (I).

In a further aspect, the present invention provides a compound of formula (I) for use in a method of treating a skin disease or skin disorder of a mammal, wherein said method comprises topical administration of said compound to said mammal, preferably to a human,

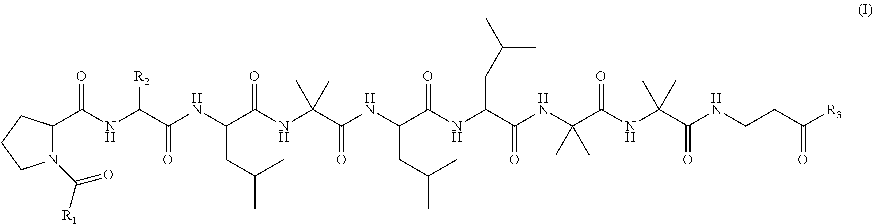

wherein
R₁ is selected from carbocyclyl or heterocyclyl, each independently optionally substituted with C₁-C₄alkyl, halogen, oxo, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl;
R₂ is selected from C₅-C₁₂alkyl, C₄-C₁₀alkoxy, C₁-C₃alkylene-cycloalkyl, C₁-C₃alkylene-aryl, C₁-C₃alkylene-heteroaryl, wherein said alkyl, cycloalkyl, aryl and heteroaryl are each independently optionally substituted with one or more, typically and preferably, one or two substituents selected from C₁-C₂alkyl, C₁-C₂haloalkyl, halogen, C₁-C₂alkoxy;
R₃ is

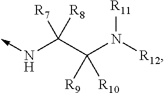

wherein
   R₇, R₈, R₉ and R₁₀ are independently at each occurrence H or C₁-C₃alkyl, preferably H or methyl, or independently at each occurrence two of said R₇, R₈, R₉ and R₁₀ together with the carbon atom to which they are attached form a carbocyclic or heterocyclic ring, preferably a carbocyclic ring, and wherein
      R₁₁ and R₁₂ are independently of each other H or C₁-C₄alkyl optionally substituted with halogen, hydroxyl or C₃-C₆cycloalkyl; or together with the nitrogen atom to which they are attached form independently at each occurrence a heteroaryl or a heterocyclyl, each independently optionally substituted with halogen, C₁-C₄alkyl, OR₁₃, NR₁₄R₁₅, wherein R₁₃, R₁₄, R₁₅ are independently at each occurrence H, C₁-C₄alkyl, and wherein the arrow indicates the attachment to the C(O)-moiety depicted in formula (I);
and pharmaceutically acceptable salts of said compound of formula (I), wherein preferably said method comprises topical administration of an effective amount of said compound to said mammal, preferably to a human.

In another aspect, the present invention provides a method for topically treating a skin disease or skin disorder of a mammal, preferably of a human, wherein said method comprises topical administration of a compound of formula (I) of the present invention, or the pharmaceutical composition comprising a compound of formula (I), to said mammal, preferably to said human, and wherein preferably said method comprises topical administration of an effective amount of said compound to said mammal, preferably to said human.

In a further aspect, the present invention provides for the use of a compound of formula (I) of the present invention, or the pharmaceutical composition comprising a compound of formula (I), for the preparation of a medicament for topically treating a skin disease or skin disorder of a mammal, preferably of a human, wherein said method comprises topical administration of said compound of formula (I), or said pharmaceutical composition of the invention, to said mammal, preferably to said human, and wherein preferably said method comprises topical administration of an effective amount of said compound, or said pharmaceutical composition, to said mammal, preferably to said human.

In a preferred embodiment, said compound of formula (I) is a compound of any one of the formula (II) to (IV)

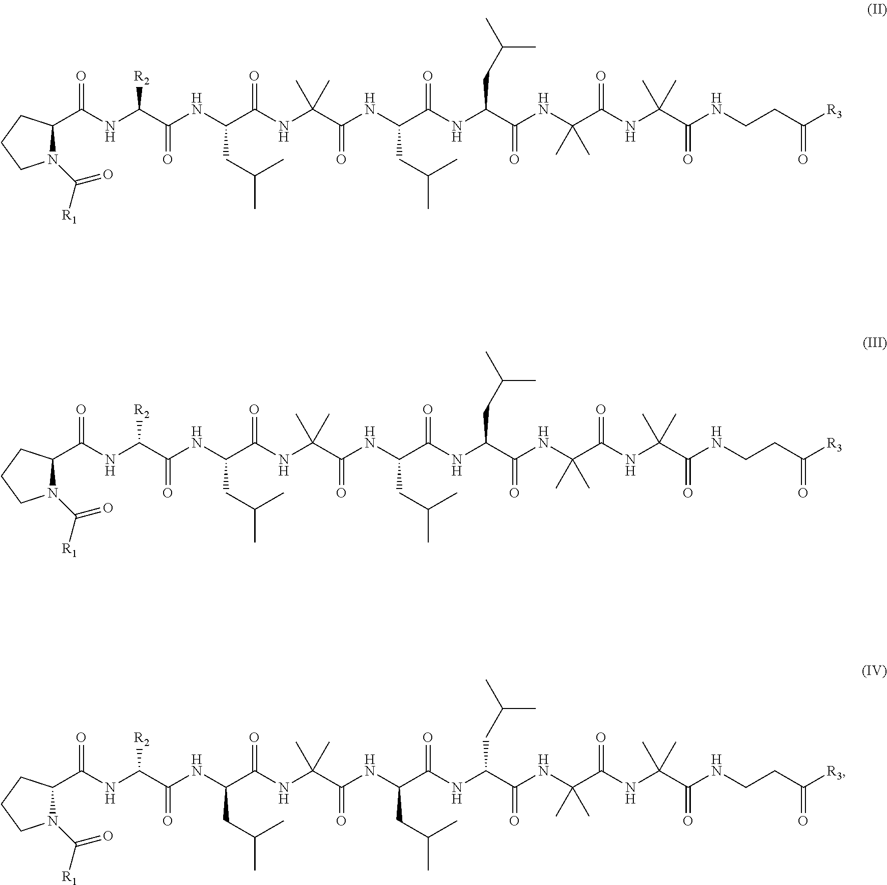

wherein preferably said compound of formula (I) is a compound of formula (II).

In another preferred embodiment, said compound of formula (I) is a compound of formula (II), formula (III) or formula (IV), wherein preferably said compound of formula (I) is a compound of formula (II). In another preferred embodiment, said compound of formula (I) is a compound of formula (II). In another preferred embodiment, said compound of formula (I) is a compound of formula (III). In another preferred embodiment, said compound of formula (I) is a compound of formula (IV).

In a further preferred embodiment, said R₁ is selected from phenyl, naphthyl, 1,2-dihydronapthalenyl, 1,2,3,4-tetrahydronaphthenyl, anthracenyl, phenanthrenyl, biphenyl, indenyl, indanyl, pyridinyl, imidazolyl, imidazopyridinyl, pyrimidinyl, pyrazolyl, triazolyl, pyrazinyl, tetrazolyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, thiadiazolyl, furazanyl, benzofurazanyl, benzopyranyl, benzothiophenyl, benzothiazolyl, benzooxazolyl, quinazolinyl, quinoxalinyl, naphthyridinyl, furopyridinyl, cyclopentyl, cyclopentenyl, cyclohexyl, cyclohexenyl, adamantanyl, each independently optionally substituted with C₁-C₄alkyl, halogen, oxo, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl.

In another preferred embodiment, said R₁ is selected from cycloalkyl, aryl or heteroaryl, each independently optionally substituted with C₁-C₄alkyl, halogen, oxo, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl.

In a further preferred embodiment, said R₁ is selected from cycloalkyl, monocyclic or bicyclic aryl or heteroaryl, each independently optionally substituted with C₁-C₄alkyl, halogen, oxo, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl. In a further preferred embodiment, said R₁ is selected from cycloalkyl, monocyclic or bicyclic aromatic aryl or heteroaryl, each independently optionally substituted with C₁-C₄alkyl, halogen, oxo, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl.

In a further preferred embodiment, said R₁ is selected from phenyl, naphthyl, 1,2-dihydronapthalenyl, 1,2,3,4-tetrahydronaphthenyl, biphenyl, indenyl, indanyl, pyridinyl, imidazolyl, imidazopyridinyl, pyrimidinyl, pyrazolyl, triazolyl, pyrazinyl, tetrazolyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, thiadiazolyl, furazanyl, benzofurazanyl, benzopyranyl, benzothiophenyl, benzothiazolyl, benzooxazolyl, quinazolinyl, quinoxalinyl, naphthyridinyl, furopyridinyl, cyclopentyl, cyclohexyl, adamantanyl, each independently optionally substituted with C₁-C₄alkyl, halogen, oxo, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl.

In a further preferred embodiment, said R₁ is selected from the formula

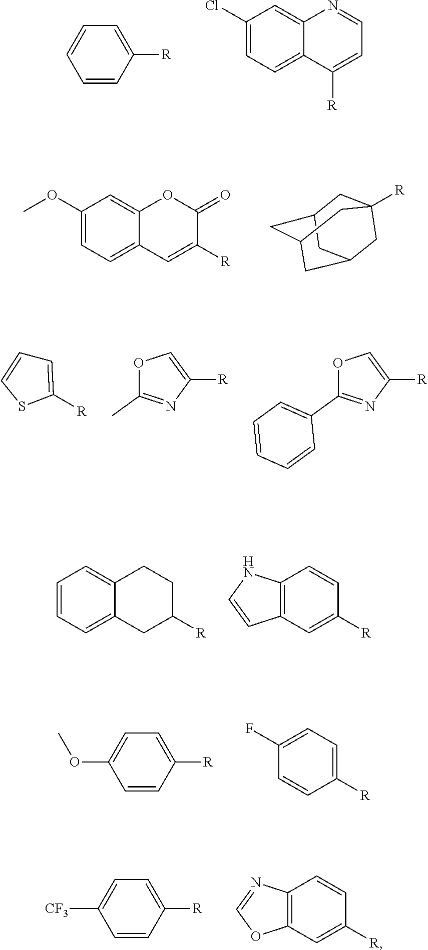

wherein R indicates the attachment to the C(O)-moiety depicted in formula (I).

In a further preferred embodiment, said R₁ is selected from phenyl or monocyclic or bicyclic heteroaryl, each independently optionally substituted with C₁-C₄alkyl, halogen, oxo, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl. In a further preferred embodiment, said R₁ is selected from phenyl or monocyclic or bicyclic aromatic heteroaryl, each independently optionally substituted with C₁-C₄alkyl, halogen, oxo, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl.

In a further preferred embodiment, said R₁ is selected from phenyl, pyridinyl, imidazolyl, imidazopyridinyl, pyrimidinyl, pyrazolyl, triazolyl, pyrazinyl, tetrazolyl, furyl, thienyl, isoxazolyl, thiazolyl, oxadiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, thiadiazolyl, benzofurazanyl, benzopyranyl, benzothiophenyl, benzothiazolyl, benzooxazolyl, each independently optionally substituted with C₁-C₄alkyl, halogen, oxo, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl.

In a further preferred embodiment, said R₁ is selected from phenyl, imidazolyl, furyl, thienyl, isoxazolyl, thiazolyl, oxazolyl, isothiazolyl, pyrrolyl, quinolinyl, isoquinolinyl, indolyl, benzimidazolyl, benzopyranyl, benzothiophenyl, benzothiazolyl, benzooxazolyl, each independently optionally substituted with C₁-C₄alkyl, halogen, oxo, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl.

In a further preferred embodiment, said R₁ is selected from phenyl, imidazolyl, furyl, thienyl, isoxazolyl, thiazolyl, oxazolyl, isothiazolyl, pyrrolyl, quinolinyl, isoquinolinyl, indolyl, benzimidazolyl, benzopyranyl, benzothiophenyl, benzothiazolyl, benzooxazolyl, each independently optionally substituted with methyl, ethyl, chlorine, fluorine, oxo, CF₃, OC₁-C₂alkyl, NR₁₁R₁₂, C₆H₅, C₆H₅ substituted with methyl, ethyl, chlorine, fluorine, OC₁-C₂alkyl, NR₁₁R₁₂, wherein RH, R₁₂ are independently at each occurrence H, methyl, ethyl.

In a further preferred embodiment, said R₁ is selected from the formula

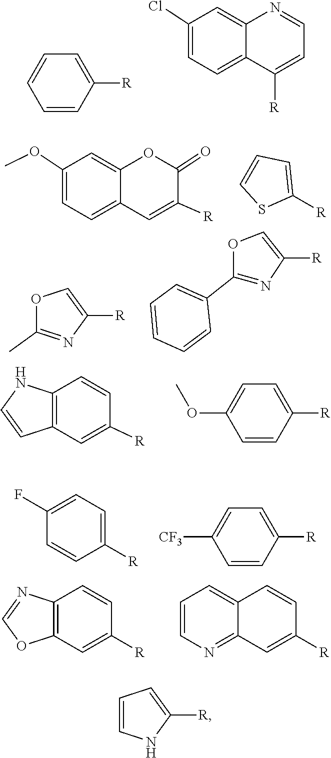

wherein R indicates the attachment to the C(O)-moiety depicted in formula (I).

In a further preferred embodiment said R₁ is selected from phenyl or a monocyclic heteroaryl, each independently optionally substituted with C₁-C₄alkyl, halogen, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl.

In a further preferred embodiment, said R₁ is selected from phenyl or a monocyclic heteroaryl, each independently optionally substituted, preferably mono-substituted, with methyl, ethyl, chlorine, fluorine, CF₃, OC₁-C₂alkyl, NR₅R₆, C₆H₅, C₆H₅ substituted with methyl, ethyl, chlorine, fluorine, OC₁-C₂alkyl, NR₅R₆, wherein R₅, R₆ are independently at each occurrence H, methyl, ethyl.

In a further preferred embodiment said R₁ is selected from phenyl or a monocyclic heteroaryl comprising one or two heteroatoms selected from N, O and S; each independently optionally substituted with C₁-C₄alkyl, halogen, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl.

In a further preferred embodiment, said R₁ is selected from phenyl or a monocyclic heteroaryl comprising one or two heteroatoms selected from N, O and S; each independently optionally substituted, preferably mono-substituted, with methyl, ethyl, chlorine, fluorine, CF₃, OC₁-C₂alkyl, NR₅R₆, C₆H₅, C₆H₅ substituted with methyl, ethyl, chlorine, fluorine, OC₁-C₂alkyl, NR₅R₆, wherein R₅, R₆ are independently at each occurrence H, methyl, ethyl.

In a further preferred embodiment, said R₁ is selected from phenyl, imidazolyl, furyl, thienyl, isoxazolyl, thiazolyl, oxazolyl, isothiazolyl, pyrrolyl, indolyl, each independently optionally substituted with C₁-C₄alkyl, halogen, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl.

In a further preferred embodiment, said R₁ is selected from phenyl, imidazolyl, furyl, thienyl, isoxazolyl, thiazolyl, oxazolyl, isothiazolyl, pyrrolyl, indolyl, each independently optionally substituted, preferably mono-substituted, with methyl, ethyl, chlorine, fluorine, CF 3, OC₁-C₂alkyl, NR₅R₆, C₆H₅, C₆H₅ substituted with methyl, ethyl, chlorine, fluorine, OC₁-C₂alkyl, NR₅R₆, wherein R₅, R₆ are independently at each occurrence H, methyl, ethyl.

In a further preferred embodiment, said R₁ is selected from phenyl, thienyl, oxazolyl, pyrrolyl, each independently optionally substituted with C₁-C₄alkyl, halogen, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl. In a further preferred embodiment, said R₁ is selected from phenyl, thienyl, oxazolyl, pyrrolyl, each independently optionally substituted, preferably mono-substituted, with methyl, ethyl, chlorine, fluorine, CF 3, OC₁-C₂alkyl, NR₅R₆, C₆H₅, C₆H₅ substituted with methyl, ethyl, chlorine, fluorine, OC₁-C₂alkyl, NR₅R₆, wherein R₅, R₆ are independently at each occurrence H, methyl, ethyl.

In a further preferred embodiment, said R₁ is selected from the formula

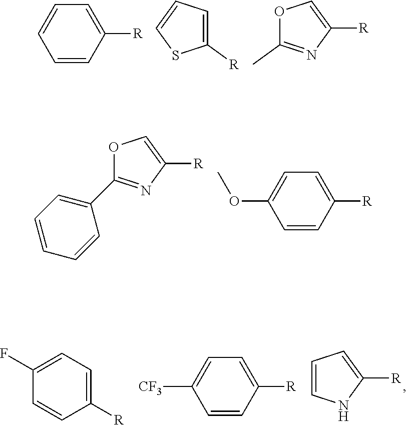

wherein R indicates the attachment to the C(O)-moiety depicted in formula (I).

In a further preferred embodiment, said R₁ is phenyl or oxazolyl, each independently optionally substituted with C₁-C₄alkyl, halogen, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl.

In a further preferred embodiment, said R₁ is phenyl or oxazolyl, each independently optionally substituted, preferably mono-substituted, with methyl, ethyl, chlorine, fluorine, CF₃, OC₁-C₂alkyl, NR₅R₆, C₆H₅, C₆H₅ substituted with methyl, ethyl, chlorine, fluorine, OC₁-C₂alkyl, NR₅R₆, wherein Rut, R₁₂ are independently at each occurrence H, methyl, ethyl.

In a further preferred embodiment, said R₁ is phenyl or oxazolyl, each independently optionally substituted, preferably mono-substituted, with methyl, chlorine, fluorine, CF₃, OCH₃ C₆H₅, C₆H₅ substituted, preferably mono-substituted, with methyl or fluorine.

In a further preferred embodiment, said R₁ is selected from the formula

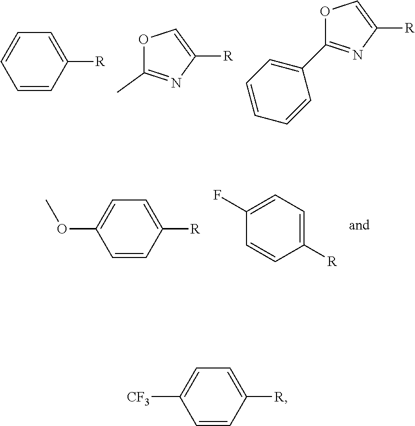

wherein R indicates the attachment to the C(O)-moiety depicted in formula (I).

In a further very preferred embodiment, said R₁ is

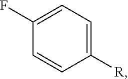

wherein R indicates the attachment to the C(O)-moiety depicted in formula (I).

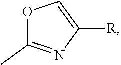

In a another very preferred embodiment, said R₁ is wherein R indicates the attachment to the C(O)-moiety depicted in formula (I).

In a another very preferred embodiment, said R₂ is selected from C₅-C₁₂alkyl, C₄-C₁₀alkoxy, C₁-C₃alkylene-cycloalkyl, C₁-C₃alkylene-aryl, C₁-C₃ alkylene-heteroaryl, wherein said alkyl, cycloalkyl, aryl and heteroaryl are each independently optionally substituted with one or more, typically and preferably one or two, substituents selected from C₁-C₂alkyl, C₁-C₂haloalkyl, halogen, C₁-C₂alkoxy.

In a another very preferred embodiment, said R₂ is selected from C₅-C₁₂alkyl, C₁₀alkoxy, C₁-C₃alkylene-C₅-C₆cycloalkyl, C₁-C₃alkylene-phenyl, C₁-C₃alkylene-(mono- or bicyclic-heteroaryl), wherein said phenyl, C₅-C₆-cycloalkyl, and mono- or bicyclic-heteroaryl are each independently optionally substituted with one or more, typically and preferably one or two, substituents selected from C₁-C₂alkyl, C₁-C₂haloalkyl, halogen, C₁-C₂alkoxy, wherein preferably said mono- or bicyclic-heteroaryl is selected from imidazolyl, furyl, thienyl, isoxazolyl, thiazolyl, oxazolyl, isothiazolyl, pyrrolyl, quinolinyl, isoquinolinyl, indolyl, benzimidazolyl, benzopyranyl, benzothiophenyl, benzothiazolyl, benzooxazolyl.

In a further preferred embodiment said R₂ is selected from C₅-C₁₂alkyl, C₄-C₁₀alkoxy, C₁-C₃alkylene-C₅-C₆cycloalkyl, C₁-C₃alkylene-phenyl, C₁-C₃alkylene-(mono- or bicyclic-heteroaryl), wherein said phenyl, C₅-C₆-cycloalkyl, and mono- or bicyclic-heteroaryl are each independently optionally substituted with one or more, typically and preferably one or two, substituents selected from methyl, ethyl, fluorine, chlorine, methoxy, wherein preferably said mono- or bicyclic-heteroaryl is selected from imidazolyl, furyl, thienyl, isoxazolyl, thiazolyl, oxazolyl, isothiazolyl, pyrrolyl, quinolinyl, isoquinolinyl, indolyl, benzimidazolyl, benzopyranyl, benzothiophenyl, benzothiazolyl, benzooxazolyl.

In a further preferred embodiment said R₂ is selected from C₅-C₁₂alkyl, C₄-C₁₀alkoxy, C₁-C₂alkylene-C₅-C₆cycloalkyl, C₁-C₂alkylene-phenyl, C₁-C₂alkylene-(mono- or bicyclic-heteroaryl), wherein said phenyl, C₅-C₆-cycloalkyl, and mono- or bicyclic-heteroaryl are each independently optionally substituted with one or more, typically and preferably one or two, substituents selected from C₁-C₂alkyl, C₁-C₂haloalkyl, halogen, C₁-C₂alkoxy, wherein preferably said mono- or bicyclic-heteroaryl is selected from imidazolyl, furyl, thienyl, isoxazolyl, thiazolyl, oxazolyl, isothiazolyl, pyrrolyl, quinolinyl, isoquinolinyl, indolyl, benzimidazolyl, benzopyranyl, benzothiophenyl, benzothiazolyl, benzooxazolyl, and wherein further preferably said mono- or bicyclic-heteroaryl is selected from isoxazolyl, thiazolyl, oxazolyl, isothiazolyl, indolyl, benzimidazolyl, benzothiazolyl, benzooxazolyl.

In a further preferred embodiment said R₂ is selected from C₅-C₁₂alkyl, C₄-C₁₀alkoxy, C₁-C₂alkylene-C₅-C₆cycloalkyl, C₁-C₂alkylene-phenyl, C₁-C₂alkylene-(mono- or bicyclic-heteroaryl), wherein said phenyl, C₅-C₆-cycloalkyl, and mono- or bicyclic-heteroaryl are each independently optionally substituted with one or more, typically and preferably one or two, substituents selected from methyl, ethyl, fluorine, chlorine, methoxy, wherein preferably said mono- or bicyclic-heteroaryl is selected from imidazolyl, furyl, thienyl, isoxazolyl, thiazolyl, oxazolyl, isothiazolyl, pyrrolyl, quinolinyl, isoquinolinyl, indolyl, benzimidazolyl, benzopyranyl, benzothiophenyl, benzothiazolyl, benzooxazolyl, and wherein further preferably said mono- or bicyclic-heteroaryl is selected from isoxazolyl, thiazolyl, oxazolyl, isothiazolyl, indolyl, benzimidazolyl, benzothiazolyl, benzooxazolyl.

In a further preferred embodiment said R₂ is selected from C₅-C₁₂alkyl, C₄-C₁₀alkoxy, C₁-C₂alkylene-C₅-C₆cycloalkyl, C₁-C₂alkylene-phenyl, C₁-C₂alkylene-(mono- or bicyclic-heteroaryl), wherein said phenyl, C₅-C₆-cycloalkyl, and mono- or bicyclic-heteroaryl are each independently optionally substituted with one or more, typically and preferably one or two, substituents selected from methyl, ethyl, fluorine, chlorine, methoxy, wherein said mono- or bicyclic-heteroaryl is selected from imidazolyl, furyl, thienyl, isoxazolyl, thiazolyl, oxazolyl, isothiazolyl, pyrrolyl, quinolinyl, isoquinolinyl, indolyl, benzimidazolyl, benzopyranyl, benzothiophenyl, benzothiazolyl, benzooxazolyl.

In a further preferred embodiment said R₂ is selected from C₅-C₁₂alkyl, C₄-C₁₀alkoxy, C₁-C₂alkylene-C₅-C₆cycloalkyl, C₁-C₂alkylene-phenyl, C₁-C₂alkylene-(mono- or bicyclic-heteroaryl), wherein said phenyl, C₅-C₆-cycloalkyl, and mono- or bicyclic-heteroaryl are each independently optionally substituted with one or more, typically and preferably one or two, substituents selected from methyl, ethyl, fluorine, chlorine, methoxy, wherein said mono- or bicyclic-heteroaryl is selected from isoxazolyl, thiazolyl, oxazolyl, isothiazolyl, indolyl, benzimidazolyl, benzothiazolyl, benzooxazolyl.

In a further preferred embodiment said R₂ is selected from C₅-C₁₂alkyl, C₄-C₁₀alkoxy, C₁-C₂alkylene-C₅-C₆cycloalkyl, CH₂-phenyl, CH₂-(mono- or bicyclic-heteroaryl), wherein said phenyl, C₅-C₆-cycloalkyl, and mono- or bicyclic-heteroaryl are each independently optionally substituted with one or more, typically and preferably one or two, substituents selected from C₁-C₂alkyl, C₁-C₂haloalkyl, halogen, C₁-C₂ alkoxy, wherein preferably said mono- or bicyclic-heteroaryl is selected from isoxazolyl, thiazolyl, oxazolyl, isothiazolyl, indolyl, benzimidazolyl, benzothiazolyl, benzooxazolyl, and wherein further preferably said mono- or bicyclic-heteroaryl is selected from, thiazolyl, indolyl, and benzothiazolyl.

In a further preferred embodiment said R₂ is selected from C₅-C₁₂alkyl, C₄-C₁₀alkoxy, C₁-C₂alkylene-C₅-C₆cycloalkyl, CH₂-phenyl, CH₂-(mono- or bicyclic-heteroaryl), wherein said phenyl, C₅-C₆-cycloalkyl, and mono- or bicyclic-heteroaryl are each independently optionally substituted with one or more, typically and preferably one or two, substituents selected from methyl, ethyl, fluorine, chlorine, methoxy, wherein preferably said mono- or bicyclic-heteroaryl is selected from isoxazolyl, thiazolyl, oxazolyl, isothiazolyl, indolyl, benzimidazolyl, benzothiazolyl, benzooxazolyl, and wherein further preferably said mono- or bicyclic-heteroaryl is selected from, thiazolyl, indolyl, and benzothiazolyl.

In a further preferred embodiment said R₂ is selected from C₅-C₁₂alkyl, C₄-C₁₀alkoxy, C₁-C₂alkylene-C₅-C₆cycloalkyl, CH₂-phenyl, CH₂-(mono- or bicyclic-heteroaryl), wherein said phenyl, C₅-C₆-cycloalkyl, and mono- or bicyclic-heteroaryl are each independently optionally substituted with one or more, typically and preferably one or two, substituents selected from methyl, ethyl, fluorine, chlorine, methoxy, wherein said mono- or bicyclic-heteroaryl is selected from isoxazolyl, thiazolyl, oxazolyl, isothiazolyl, indolyl, benzimidazolyl, benzothiazolyl, benzooxazolyl.

In a further preferred embodiment said R₂ is selected from C₅-C₁₂alkyl, C₄-C₁₀alkoxy, C₁-C₂alkylene-C₅-C₆cycloalkyl, CH₂-phenyl, CH₂-(mono- or bicyclic-heteroaryl), wherein said phenyl, C₅-C₆-cycloalkyl, and mono- or bicyclic-heteroaryl are each independently optionally substituted with one or more, typically and preferably one or two, substituents selected from methyl, ethyl, fluorine, chlorine, methoxy, wherein said mono- or bicyclic-heteroaryl is selected from, thiazolyl, indolyl, and benzothiazolyl.

In a further preferred embodiment said R₂ is selected from

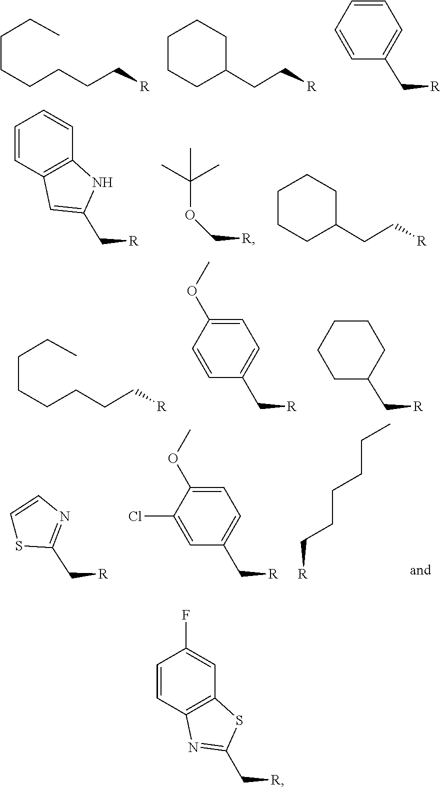

wherein R indicates the attachment to the CH-moiety depicted in formula (I).

In a further preferred embodiment, said R₂ is selected from C₅-C₁₂alkyl, C₁-C₂alkylene-C₅-C₆cycloalkyl and CH₂-phenyl, wherein said phenyl is optionally substituted with one or two substituents selected from methyl, ethyl, fluorine, chlorine and methoxy.

In a further preferred embodiment, said R₂ is selected from

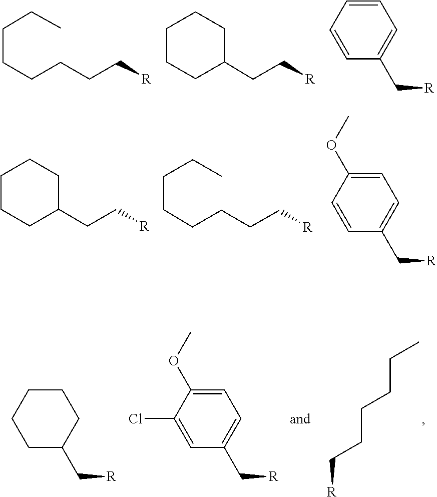

wherein R indicates the attachment to the CH-moiety depicted in formula (I).

In a further preferred embodiment, said R₂ is selected from C₅-C₁₂alkyl and C₁-C₂alkylene-C₅-C₆cycloalkyl.

In a further preferred embodiment, said R₂ is selected from

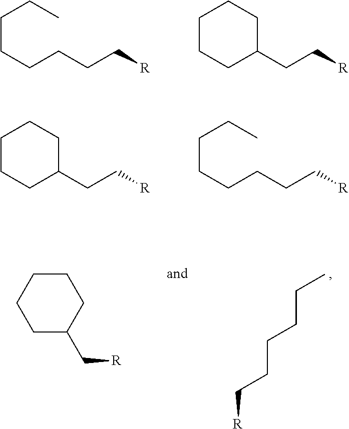

wherein R indicates the attachment to the CH-moiety depicted in formula (I).

In a further very preferred embodiment, said R₂ is

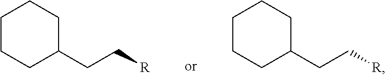

wherein R indicates the attachment to the CH-moiety depicted in formula (I).

In a further very preferred embodiment, said R₂ is

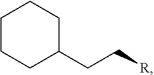

wherein R indicates the attachment to the CH-moiety depicted in formula (I).

In a further preferred embodiment, said R₃ is

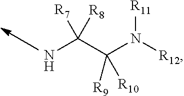

wherein
R₇, R₈, R₉ and R₁₀ are independently at each occurrence H or C₁-C₃alkyl, preferably H or methyl, or independently at each occurrence two of said R₇, R₈, R₉ and R₁₀ together with the carbon atom to which they are attached form a carbocyclic ring, and wherein
   R₁₁ and R₁₂ are independently of each other H or C₁-C₄alkyl optionally substituted with halogen, hydroxyl or C₃-C₆cycloalkyl; or together with the nitrogen atom to which they are attached form independently at each occurrence a heteroaryl or a heterocyclyl, each independently optionally substituted with halogen, C₁-C₄alkyl, OR₁₃, NR₁₄R₁₅; wherein R₁₃, R₁₄, R₁₅ are independently at each occurrence H, C₁-C₄alkyl, and wherein the arrow indicates the attachment to the C(O)-moiety depicted in formula (I).

In a further preferred embodiment, said R₇, R₈, R₉ and R₁₀ are independently at each occurrence H or C₁-C₃alkyl, preferably H or methyl, or independently at each occurrence two of said R₇, Its, R₉ and R₁₀ together with the carbon atom to which they are attached form a carbocyclic or heterocyclic ring, preferably a carbocyclic ring, and wherein RH and R₁₂ are independently of each other H or C₁-C₄alkyl optionally substituted with halogen, hydroxyl or C₃-C₆cycloalkyl; or together with the nitrogen atom to which they are attached form independently at each occurrence a mono- or bicyclic heteroaryl or a mono- or bicyclic heterocyclyl, preferably selected from a pyridinyl, imidazolyl, imidazopyridinyl, pyrimidinyl, pyrazolyl, triazolyl, pyrazinyl, tetrazolyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, indazolyl, indolizinyl, pyridazinyl, triazinyl, isoindolyl, purinyl, pyrrolidinyl, piperidinyl, morpholinyl, thiomorpholinyl, piperazinyl, homopiperazinyl, 2-pyrrolinyl, 3-pyrrolinyl, indolinyl, dihyrooxazolyl, pyrazolinyl, pyrazolidinyl, imidazolinyl, imidazolidinyl, each independently optionally substituted with halogen, C₁-C₄alkyl, OR₁₃, NR₁₄R₁₅; wherein R₁₃, R₁₄, R₁₅ are independently at each occurrence H, C₁-C₄alkyl.

In a further preferred embodiment, R₇, R₈, R₉ and R₁₀ are independently at each occurrence H or C₁-C₃alkyl, preferably H or methyl, or independently at each occurrence two of said R₇, R₈, R₉ and R₁₀ together with the carbon atom to which they are attached form a monocyclic carbocyclic or monocyclic heterocyclic ring, preferably a monocyclic carbocyclic ring, wherein further preferably said monocyclic carbocyclic ring is selected from cyclopropyl, cyclobutyl, cyclopentyl or cyclohexyl, and wherein again further preferably said monocyclic carbocyclic ring is cyclobutyl or cyclopentyl, and wherein R₁₁ and R₁₂ are independently of each other H or C₁-C₄alkyl optionally substituted with halogen, hydroxyl or C₃-C₆cycloalkyl; or together with the nitrogen atom to which they are attached form independently at each occurrence a mono- or bicyclic heteroaryl or a a mono- or bicyclic heterocyclyl, preferably selected from a pyridinyl, imidazolyl, imidazopyridinyl, pyrimidinyl, pyrazolyl, triazolyl, pyrazinyl, tetrazolyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, indazolyl, indolizinyl, pyridazinyl, triazinyl, isoindolyl, purinyl, pyrrolidinyl, piperidinyl, morpholinyl, thiomorpholinyl, piperazinyl, homopiperazinyl, 2-pyrrolinyl, 3-pyrrolinyl, indolinyl, dihyrooxazolyl, pyrazolinyl, pyrazolidinyl, imidazolinyl, imidazolidinyl, each independently optionally substituted with halogen, C₁-C₄alkyl, OR₁₃, NR₁₄R₁₅; wherein R₁₃, RN, R₁₅ are independently at each occurrence H, C₁-C₄alkyl.

In a further preferred embodiment, R₇, R₈, R₉ and R₁₀ are independently at each occurrence H or C₁-C₃alkyl, preferably H or methyl, or independently at each occurrence two of said R₇, R₈, R₉ and R₁₀ together with the carbon atom to which they are attached form a monocyclic carbocyclic or monocyclic heterocyclic ring, preferably a monocyclic carbocyclic ring, wherein further preferably said monocyclic carbocyclic ring is selected from cyclopropyl, cyclobutyl, cyclopentyl or cyclohexyl, and wherein again further preferably said monocyclic carbocyclic ring is cyclobutyl or cyclopentyl, and wherein R₁₁ and R₁₂ are independently of each other H or C₁-C₄alkyl optionally substituted with halogen, hydroxyl or C₃-C₆cycloalkyl;

or together with the nitrogen atom to which they are attached form independently at each occurrence a monocyclic heteroaryl or a monocyclic heterocyclyl, each independently optionally substituted with halogen, C₁-C₄alkyl, OR₁₃, NR₁₄R₁₅; wherein R₁₃, R₁₄, R₁₅ are independently at each occurrence H, C₁-C₄alkyl.

In a further preferred embodiment, R₇, R₈, R₉ and R₁₀ are independently at each occurrence H or C₁-C₃alkyl, preferably H or methyl, or independently at each occurrence two of said R₇, R₈, R₉ and R₁₀ together with the carbon atom to which they are attached form a monocyclic carbocyclic or monocyclic heterocyclic ring, preferably a monocyclic carbocyclic ring, wherein further preferably said monocyclic carbocyclic ring is selected from cyclopropyl, cyclobutyl, cyclopentyl or cyclohexyl, and wherein again further preferably said monocyclic carbocyclic ring is cyclobutyl or cyclopentyl, and wherein RH and R₁₂ are independently of each other H or C₁-C₄alkyl optionally substituted with halogen, hydroxyl or C₃-C₆cycloalkyl; or together with the nitrogen atom to which they are attached form independently at each occurrence a monocyclic heteroaryl or a monocyclic heterocyclyl, wherein said monocyclic heteroaryl or said monocyclic heterocyclyl comprise one or two heteroatoms (including said nitrogen atom to which RH and R₁₂ are attached) selected from nitrogen, oxygen and sulphur, each independently optionally substituted with halogen, C₁-C₄alkyl, OR₁₃, NR₁₄R₁₅; wherein R₁₃, R₁₄, R₁₅ are independently at each occurrence H, C₁-C₄alkyl.

In a further preferred embodiment, R₇, R₈, R₉ and R₁₀ are independently at each occurrence H or C₁-C₃alkyl, preferably H or methyl, or independently at each occurrence two of said R₇, R₈, R₉ and R₁₀ together with the carbon atom to which they are attached form a monocyclic carbocyclic or monocyclic heterocyclic ring, preferably a monocyclic carbocyclic ring, wherein further preferably said monocyclic carbocyclic ring is selected from cyclopropyl, cyclobutyl, cyclopentyl or cyclohexyl, and wherein again further preferably said monocyclic carbocyclic ring is cyclobutyl or cyclopentyl, and wherein RH and R₁₂ are independently of each other H or C₁-C₄alkyl optionally substituted with halogen, hydroxyl or C₃-C₆cycloalkyl; or together with the nitrogen atom to which they are attached form independently at each occurrence a monocyclic heteroaryl or a monocyclic heterocyclyl selected from a pyridinyl, imidazolyl, pyrimidinyl, pyrazolyl, triazolyl, pyrazinyl, tetrazolyl, isoxazolyl, thiazolyl, oxadiazolyl, oxazolyl, isothiazolyl, pyrrolyl, pyridazinyl, triazinyl, pyrrolidinyl, piperidinyl, morpholinyl, thiomorpholinyl, piperazinyl, 2-pyrrolinyl, 3-pyrrolinyl, dihyrooxazolyl, pyrazolinyl, pyrazolidinyl, imidazolinyl, imidazolidinyl, each independently optionally substituted with halogen, C₁-C₄alkyl, OR₁₃, NR₁₄R₁₅; wherein R₁₃, R₁₄, R₁₅ are independently at each occurrence H, C₁-C₄alkyl.

In a further preferred embodiment, R₇, R₈, R₉ and R₁₀ are independently at each occurrence H or C₁-C₃alkyl, preferably H or methyl, or independently at each occurrence two of said R₇, R₈, R₉ and R₁₀ together with the carbon atom to which they are attached form a monocyclic carbocyclic ring, wherein further preferably said monocyclic carbocyclic ring is selected from cyclopropyl, cyclobutyl, cyclopentyl or cyclohexyl, and wherein again further preferably said monocyclic carbocyclic ring is cyclobutyl or cyclopentyl, and wherein R₁₁ and R₁₂ are independently of each other H or C₁-C₄alkyl optionally substituted with halogen, hydroxyl or C₃-C₆cycloalkyl; or together with the nitrogen atom to which they are attached form independently at each occurrence a monocyclic heterocyclic ring, each independently optionally substituted with halogen, C₁-C₄alkyl, OR₁₃, NR₁₄R₁₅; wherein Rn, R₁₀, R₁₅ are independently at each occurrence H, C₁-C₄alkyl.

In a further preferred embodiment, R₇, R₈, R₉ and R₁₀ are independently at each occurrence H or C₁-C₃alkyl, preferably H or methyl, or independently at each occurrence two of said R₇, R₈, R₉ and R₁₀ together with the carbon atom to which they are attached form a monocyclic carbocyclic or monocyclic heterocyclic ring, preferably a monocyclic carbocyclic ring, wherein further preferably said monocyclic carbocyclic ring is selected from cyclopropyl, cyclobutyl, cyclopentyl or cyclohexyl, and wherein again further preferably said monocyclic carbocyclic ring is cyclobutyl or cyclopentyl, and wherein RH and R₁₂ are independently of each other H or C₁-C₄alkyl optionally substituted with halogen, hydroxyl or C₃-C₆cycloalkyl; or together with the nitrogen atom to which they are attached form independently at each occurrence a monocyclic heterocyclyl, wherein said monocyclic heterocyclyl comprise one or two heteroatoms (including said nitrogen atom to which R₁₁ and R₁₂ are attached) selected from nitrogen, oxygen and sulphur, each independently optionally substituted with halogen, C₁-C₄alkyl, OR₁₃, NR₁₄R₁₅; wherein R₁₃, R₁₄, R₁₅ are independently at each occurrence H, C₁-C₄alkyl.

In a further preferred embodiment, R₇, R₈, R₉ and R₁₀ are independently at each occurrence H or C₁-C₃alkyl, preferably H or methyl, or independently at each occurrence two of said R₇, R₈, R₉ and R₁₀ together with the carbon atom to which they are attached form a monocyclic carbocyclic ring, wherein further preferably said monocyclic carbocyclic ring is selected from cyclopropyl, cyclobutyl, cyclopentyl or cyclohexyl, and wherein again further preferably said monocyclic carbocyclic ring is cyclobutyl or cyclopentyl, and wherein RH and R₁₂ are independently of each other H or C₁-C₄alkyl optionally substituted with halogen, hydroxyl or C₃-C₆cycloalkyl; or together with the nitrogen atom to which they are attached form independently at each occurrence a monocyclic heterocyclic ring selected from imidazolyl, pyrimidinyl, pyrazolyl, triazolyl, pyrazinyl, tetrazolyl, isoxazolyl, thiazolyl, oxadiazolyl, oxazolyl, isothiazolyl, pyrrolyl, pyridazinyl, triazinyl, pyrrolidinyl, piperidinyl, morpholinyl, thiomorpholinyl, piperazinyl, 2-pyrrolinyl, 3-pyrrolinyl, dihyrooxazolyl, pyrazolinyl, pyrazolidinyl, imidazolinyl, imidazolidinyl, each independently optionally substituted with halogen, C₁-C₄alkyl, OR₁₃, NR₁₄R₁₅; wherein R₁₃, R₁₄, R₁₅ are independently at each occurrence H, C₁-C₄alkyl.

In a further preferred embodiment, R₇, R₈, R₉ and R₁₀ are independently at each occurrence H or C₁-C₃alkyl, preferably H or methyl, or independently at each occurrence two of said R₇, R₈, R₉ and R₁₀ together with the carbon atom to which they are attached form a monocyclic carbocyclic ring selected from cyclopropyl, cyclobutyl, cyclopentyl or cyclohexyl, preferably said monocyclic carbocyclic ring is cyclobutyl or cyclopentyl, and wherein R₁₁ and R₁₂ are independently of each other H or C₁-C₄alkyl optionally substituted with halogen, hydroxyl or C₃-C₆cycloalkyl; or together with the nitrogen atom to which they are attached form independently at each occurrence a monocyclic heterocyclic ring selected from piperidinyl, morpholinyl, thiomorpholinyl, preferably from piperidinyl or morpholinyl, and further preferably from morpholinyl, each independently optionally substituted with halogen, C₁-C₄alkyl, OR₁₃, NR₁₄R₁₅; wherein R₁₃, R₁₄, R₁₅ are independently at each occurrence H, C₁-C₄alkyl.

In a further preferred embodiment, said R₃ is selected from

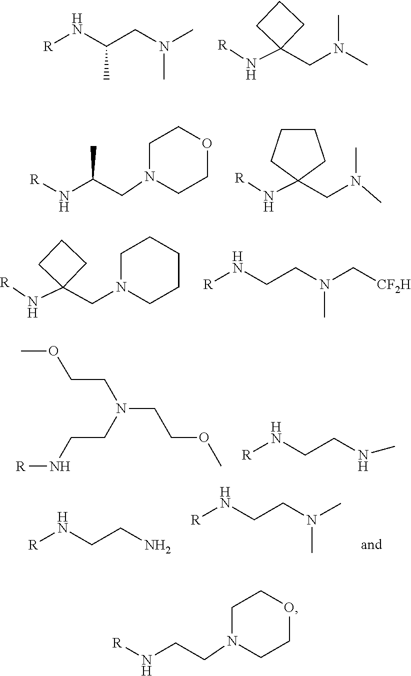

wherein R indicates the attachment to the C(O)-moiety depicted in formula (I).

In a further very preferred embodiment, said compound of formula (I) is selected from
5469: (2S)-2-{[(2S)-1-benzoylpyrrolidin-2-yl]formamido}-N-[(1S)-1-[(1-{[(1S)-1-{[(1S)-1-{[1-({1-[(2-{[(2S)-1-(dimethylamino)propan-2-yl]carbamoyl}ethyl)carbamoyl]-1-methylethyl}carbamoyl)-1-methylethyl]carbamoyl}-3-methylbutyl]carbamoyl}-3-methylbutyl]carbamoyl}-1-methylethyl)carbamoyl]-3-methylbutyl]decanamide;
5768: (2S)-2-{[(2S)-1-(7-chloroquinoline-4-carbonyl)pyrrolidin-2-yl]formamido}-N-[(1S)-1-[(1-{[(1S)-1-{[(1S)-1-{[1-({1-[(2-{[(2S)-1-(dimethylamino)propan-2-yl]carbamoyl}ethyl)carbamoyl]-1-methylethyl}carbamoyl)-1-methylethyl]carbamoyl}-3-methylbutyl]carbamoyl}-3-methylbutyl]carbamoyl}-1-methylethyl)carbamoyl]-3-methylbutyl]decanamide;
5769: (2S)-N-[(1S)-1-[(1-{[(1S)-1-{[(1S)-1-{[1-({1-[(2-{[(2S)-1-(dimethylamino)propan-2-yl]carbamoyl}ethyl)carbamoyl]-1-methylethyl}carbamoyl)-1-methylethyl]carbamoyl}-3-methylbutyl]carbamoyl}-3-methylbutyl]carbamoyl}-1-methylethyl)carbamoyl]-3-methylbutyl]-2-{[(2S)-1-(7-methoxy-2-oxo-2H-chromene-3-carbonyl)pyrrolidin-2-yl]formamido}decanamide;
5770: (2S)-2-{[(2S)-1-(adamantane-1-carbonyl)pyrrolidin-2-yl]formamido}-N-[(1S)-1-[(1-{[(1S)-1-{[(1S)-1-{[1-({1-[(2-{[(2S)-1-(dimethylamino)propan-2-yl]carbamoyl}ethyl)carbamoyl]-1-methylethyl}carbamoyl)-1-methylethyl]carbamoyl}-3-methylbutyl]carbamoyl}-3-methylbutyl]carbamoyl}-1-methylethyl)carbamoyl]-3-methylbutyl]decanamide;
5904: (2S)-N-[(1S)-1-[(1-{[(1S)-1-{[(1S)-1-{[1-({1-[(2-{[(2S)-1-(dimethylamino)propan-2-yl]carbamoyl}ethyl)carbamoyl]-1-methylethyl}carbamoyl)-1-methylethyl]carbamoyl}-3-methylbutyl]carbamoyl}-3-methylbutyl]carbamoyl}-1-methylethyl)carbamoyl]-3-methylbutyl]-2-{[(2S)-1-(thiophene-2-carbonyl)pyrrolidin-2-yl]formamido}decanamide;
5905: (2S)-N-[(1S)-1-[(1-{[(1S)-1-{[(1S)-1-{[1-({1-[(2-{[(2S)-1-(dimethylamino)propan-2-yl]carbamoyl}ethyl)carbamoyl]-1-methylethyl}carbamoyl)-1-methylethyl]carbamoyl}-3-methylbutyl]carbamoyl}-3-methylbutyl]carbamoyl}-1-methylethyl)carbamoyl]-3-methylbutyl]-2-{[(2S)-1-(2-methyl-1,3-oxazole-4-carbonyl)pyrrolidin-2-yl]formamido}decanamide;
5906: (2S)-N-[(1S)-1-[(1-{[(1S)-1-{[(1S)-1-{[1-({1-[(2-{[(2S)-1-(dimethylamino)propan-2-yl]carbamoyl}ethyl)carbamoyl]-1-methylethyl}carbamoyl)-1-methylethyl]carbamoyl}-3-methylbutyl]carbamoyl}-3-methylbutyl]carbamoyl}-1-methylethyl)carbamoyl]-3-methylbutyl]-2-{[(2S)-1-(2-phenyl-1,3-oxazole-4-carbonyl)pyrrolidin-2-yl]formamido}decanamide;
5910: (2S)—N-[(1S)-1-[(1-{[(1S)-1-{[(1S)-1-{[1-({1-[(2-{[(2S)-1-(dimethylamino)propan-2-yl]carbamoyl}ethyl)carbamoyl]-1-methylethyl}carbamoyl)-1-methylethyl]carbamoyl}-3-methylbutyl]carbamoyl}-3-methylbutyl]carbamoyl}-1-methylethyl)carbamoyl]-3-methylbutyl]-2-{[(2S)-1-(1,2,3,4-tetrahydronaphthalene-2-carbonyl)pyrrolidin-2-yl]formamido}decanamide;
5911: (2S)—N-[(1S)-1-[(1-{[(1S)-1-{[(1S)-1-{[1-({1-[(2-{[(2S)-1-(dimethylamino)propan-2-yl]carbamoyl}ethyl)carbamoyl]-1-methylethyl}carbamoyl)-1-methylethyl]carbamoyl}-3-methylbutyl]carbamoyl}-3-methylbutyl]carbamoyl}-1-methylethyl)carbamoyl]-3-methylbutyl]-2-{[(2S)-1-(1H-indole-5-carbonyl)pyrrolidin-2-yl]formamido}decanamide;
5912: (2S)—N-[(1S)-1-[(1-{[(1S)-1-{[(1S)-1-{[1-({1-[(2-{[(2S)-1-(dimethylamino)propan-2-yl]carbamoyl}ethyl)carbamoyl]-1-methylethyl}carbamoyl)-1-methylethyl]carbamoyl}-3-methylbutyl]carbamoyl}-3-methylbutyl]carbamoyl}-1-methylethyl)carbamoyl]-3-methylbutyl]-2-{[(2S)-1-(4-methoxybenzoyl)pyrrolidin-2-yl]formamido}decanamide;
5934: (2S)-N-[(1S)-1-[(1-{[(1S)-1-{[(1S)-1-{[1-({1-[(2-{[(2S)-1-(dimethylamino)propan-2-yl]carbamoyl}ethyl)carbamoyl]-1-methylethyl}carbamoyl)-1-methylethyl]carbamoyl}-3-methylbutyl]carbamoyl}-3-methylbutyl]carbamoyl}-1-methylethyl)carbamoyl]-3-methylbutyl]-2-{[(2S)-1-(4-fluorobenzoyl)pyrrolidin-2-yl]formamido}decanamide;
5936: (2S)-N-[(1S)-1-[(1-{[(1S)-1-{[(1S)-1-{[1-({1-[(2-{[(2S)-1-(dimethylamino)propan-2-yl]carbamoyl}ethyl)carbamoyl]-1-methylethyl}carbamoyl)-1-methylethyl]carbamoyl}-3-methylbutyl]carbamoyl}-3-methylbutyl]carbamoyl}-1-methylethyl)carbamoyl]-3-methylbutyl]-2-{[(2S)-1-[4-(trifluoromethyl)benzoyl]pyrrolidin-2-yl]formamido}decanamide;
5937: (2S)-2-{[(2S)-1-(1,3-benzoxazole-5-carbonyl)pyrrolidin-2-yl]formamido}-N-[(1S)-1-[(1-{[(1S)-1-{[(1S)-1-{[1-({1-[(2-{[(2S)-1-(dimethylamino)propan-2-yl]carbamoyl}ethyl)carbamoyl]-1-methylethyl}carbamoyl)-1-methylethyl]carbamoyl}-3-methylbutyl]carbamoyl}-3-methylbutyl]carbamoyl}-1-methylethyl)carbamoyl]-3-methylbutyl]decanamide;
5938: (2S)-N-[(1S)-1-[(1-{[(1S)-1-{[(1S)-1-{[1-({1-[(2-{[(2S)-1-(dimethylamino)propan-2-yl]carbamoyl}ethyl)carbamoyl]-1-methylethyl}carbamoyl)-1-methylethyl]carbamoyl}-3-methylbutyl]carbamoyl}-3-methylbutyl]carbamoyl}-1-methylethyl)carbamoyl]-3-methylbutyl]-2-{[(2S)-1-(quinoline-7-carbonyl)pyrrolidin-2-yl]formamido}decanamide;
5939: (2S)-N-[(1S)-1-[(1-{[(1S)-1-{[(1S)-1-{[1-({1-[(2-{[(2S)-1-(dimethylamino)propan-2-yl]carbamoyl}ethyl)carbamoyl]-1-methylethyl}carbamoyl)-1-methylethyl]carbamoyl}-3-methylbutyl]carbamoyl}-3-methylbutyl]carbamoyl}-1-methylethyl)carbamoyl]-3-methylbutyl]-2-{[(2S)-1-(1H-pyrrole-2-carbonyl)pyrrolidin-2-yl]formamido}decanamide;
6025: (2S)-2-[(2S)-4-cyclohexyl-2-{[(2S)-1-(2-methyl-1,3-oxazole-4-carbonyl)pyrrolidin-2-yl]formamido}butanamido]-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide;
6026: (2S)-2-[(2S)-2-{2-[(2S)-2-[(2S)-4-cyclohexyl-2-{[(2S)-1-(2-methyl-1,3-oxazole-4-carbonyl)pyrrolidin-2-yl]formamido}butanamido]-4-methylpentanamido]-2-methylpropanamido}-4-methylpentanamido]-4-methyl-N-[1-methyl-1-({1-methyl-1-[(2-{[(2S)-1-(morpholin-4-yl)propan-2-yl]carbamoyl}ethyl)carbamoyl]ethyl}carbamoyl)ethyl]pentanamide;
6027: (2S)-2-[(2S)-4-cyclohexyl-2-{[(2S)-1-(4-fluorobenzoyl)pyrrolidin-2-yl]formamido}butanamido]-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide;
6028: (2S)-2-[(2S)-2-{2-[(2S)-2-[(2S)-4-cyclohexyl-2-{[(2S)-1-(4-fluorobenzoyl)pyrrolidin-2-yl]formamido}butanamido]-4-methylpentanamido]-2-methylpropanamido}-4-methylpentanamido]-4-methyl-N-[1-methyl-1-({1-methyl-1-[(2-{[(2S)-1-(morpholin-4-yl)propan-2-yl]carbamoyl}ethyl)carbamoyl]ethyl}carbamoyl)ethyl]pentanamide,
6253: (2S)-N-[(1S)-1-[(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)carbamoyl]-3-methylbutyl]-2-{[(2S)-1-(2-methyl-1,3-oxazole-4-carbonyl)pyrrolidin-2-yl]formamido}decanamide;
6254: (2S)-N-[(1S)-1-[(1-{[(1S)-1-{[(1S)-1-({1-[(1-{[2-({1-[(dimethylamino)methyl]cyclopentyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)carbamoyl]-3-methylbutyl]-2-{[(2S)-1-(2-methyl-1,3-oxazole-4-carbonyl)pyrrolidin-2-yl]formamido}decanamide;
6328: (2S)-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methyl-2-[(2S)-2-{[(2S)-1-(2-methyl-1,3-oxazole-4-carbonyl)pyrrolidin-2-yl]formamido}-3-phenylpropanamido]pentanamide;
6483: (2S)-2-[(2S)-4-cyclohexyl-2-{[(2S)-1-(2-methyl-1,3-oxazole-4-carbonyl)pyrrolidin-2-yl]formamido}butanamido]-4-methyl-N-(1-methyl-1-{[(1S)-3-methyl-1-{[(1S)-3-methyl-1-({1-methyl-1-[(1-methyl-1-{[2-({1-[(piperidin-1-yl)methyl]cyclobutyl}carbamoyl)ethyl]carbamoyl}ethyl)carbamoyl]ethyl}carbamoyl)butyl]carbamyl}butyl]carbamoyl}ethyl)pentanamide;
6488: (2S)-2-[(2S)-4-cyclohexyl-2-{[(2S)-1-(2-methyl-1,3-oxazole-4-carbonyl)pyrrolidin-2-yl]formamido}butanamido]-N-(1-{[(1S)-1-{[(1S)-1-({1-[(1-{[2-({2-[(2,2-difluoroethyl)(methyl)amino]ethyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide;
6779: (2S)-N-(1-{[(1S)-1-{[(1 S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-2-[(2S)-3-(1H-indol-3-yl)-2-{[(2S)-1-(2-methyl-1,3-oxazole-4-carbonyl)pyrrolidin-2-yl]formamido}propanamido]-4-methylpentanamide;
6780: (2S)-2-[(2S)-2-{2-[(2S)-2-[(2S)-3-(tert-butoxy)-2-{[(2S)-1-(2-methyl-1,3-oxazole-4-carbonyl)pyrrolidin-2-yl]formamido}propanamido]-4-methylpentanamido]-2-methylpropanamido}-4-methylpentanamido]-N-{1-[(1-{[2-({1-[(dimethylamino)methyl]cyclobutyl}carbamoyl)ethyl]carbamoyl}-1-methylethyl)carbamoyl]-1-methylethyl}-4-methylpentanamide;
6789: (2R)-N-[(1S)-1-[(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)carbamoyl]-3-methylbutyl]-2-{[(2S)-1-(2-methyl-1,3-oxazole-4-carbonyl)pyrrolidin-2-yl]formamido}decanamide;
6791: (2S)-N-(1-{[(1S)-1-{[(1S)-1-({1-[(1-{[2-({2-[bis(2-methoxyethyl)amino]ethyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-2-[(2S)-4-cyclohexyl-2-{[(2S)-1-(2-methyl-1,3-oxazole-4-carbonyl)pyrrolidin-2-yl]formamido}butanamido]-4-methylpentanamide;
6894: (2S)—N-[(1S)-1-[(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)carbamoyl]-3-methylbutyl]-2-{[(2S)-1-(2-methyl-1,3-oxazole-4-carbonyl)pyrrolidin-2-yl]formamido}octanamide;
6896: (2S)-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-2-[(2S)-3-(4-methoxyphenyl)-2-{[(2S)-1-(2-methyl-1,3-oxazole-4-carbonyl)pyrrolidin-2-yl]formamido}propanamido]-4-methylpentanamide;
7050: (2R)-2-[(2R)-4-cyclohexyl-2-{[(2R)-1-(2-methyl-1,3-oxazole-4-carbonyl)pyrrolidin-2-yl]formamido}butanamido]-N-(1-{[(1R)-1-{[(1R)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide;
7058: (2S)-2-{[(2S)-1-(2-methyl-1,3-oxazole-4-carbonyl)pyrrolidin-2-yl]formamido}-N-[(1S)-3-methyl-1-[(1-methyl-1-{[(1S)-3-methyl-1-{[(1S)-3-methyl-1-{[1-methyl-1-({1-methyl-1-[(2-{[2-(methylamino)ethyl]carbamoyl}ethyl)carbamoyl]ethyl}carbamoyl) ethyl]carbamoyl}butyl]carbamoyl}butyl]carbamoyl}ethyl)carbamoyl]butyl]decanamide;
7059: (2S)-2-[(2S)-3-cyclohexyl-2-{[(2S)-1-(2-methyl-1,3-oxazole-4-carbonyl)pyrrolidin-2-yl]formamido}propanamido]-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide;
7192: (2S)-N-(1-{[(1S)-1-{[(1S)-1-[(1-{[1-({2-[(2-aminoethyl)carbamoyl]ethyl}carbamoyl)-1-methylethyl]carbamoyl}-1-methylethyl)carbamoyl]-3-methylbutyl]carbamoyl}-3-methylbutyl]carbamoyl}-1-methylethyl)-2-[(2S)-4-cyclohexyl-2-{[(2S)-1-(2-methyl-1,3-oxazole-4-carbonyl)pyrrolidin-2-yl]formamido}butanamido]-4-methylpentanamide;
7842: (2S)-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-2-[(2S)-2-{[(2S)-1-(4-fluorobenzoyl)pyrrolidin-2-yl]formamido}-3-(1,3-thiazol-2-yl)propanamido]-4-methylpentanamide;
7846: (2S)-2-[(2S)-3-(3-chloro-4-methoxyphenyl)-2-{[(2S)-1-(4-fluorobenzoyl)pyrrolidin-2-yl]formamido}propanamido]-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide;
7848: (2S)-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-2[(2S)-3-(6-fluoro-1,3-benzothiazol-2-yl)-2-{[(2S)-1-(4-fluorobenzoyl)pyrrolidin-2-yl]formamido}propanamido]-4-methylpentanamide;
8341: (2S)-2-[(2S)-4-cyclohexyl-2-{[(2S)-1-(4-fluorobenzoyl)pyrrolidin-2-yl]formamido}butanamido]-4-methyl-N-(1-methyl-1-{[(1S)-3-methyl-1-{[(1S)-3-methyl-1-{[1-methyl-1-({1-methyl-1-[(2-{[2-(morpholin-4-yl)ethyl]carbamoyl}ethyl)carbamoyl]ethyl}carbamoyl)ethyl]carbamoyl}butyl]carbamoyl}butyl]carbamoyl}ethyl)pentanamide;
8342: (2S)-2-[(2S)-4-cyclohexyl-2-{[(2S)-1-(4-fluorobenzoyl)pyrrolidin-2-yl]formamido}butanamido]-N-(1-{[(1S)-1-{[(1S)-1-({1-[(1-{[2-({2-[(2,2-difluoroethyl)(methyl)amino]ethyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide;
8343: (2S)-2-[(2S)-4-cyclohexyl-2-{[(2S)-1-(4-fluorobenzoyl)pyrrolidin-2-yl]formamido}butanamido]-N-(1-{[(1S)-1-{[(1S)-1-{[1-({1-[(2-{[2-(dimethylamino)ethyl]carbamoyl}ethyl)carbamoyl]-1-methylethyl}carbamoyl)-1-methylethyl]carbamoyl}-3-methylbutyl]carbamoyl}-3-methylbutyl]carbamoyl}-1-methylethyl)-4-methylpentanamide; and wherein preferably said compound of formula (I) is
6025: (2S)-2-[(2S)-4-cyclohexyl-2-{[(2S)-1-(2-methyl-1,3-oxazole-4-carbonyl)pyrrolidin-2-yl]formamido}butanamido]-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide; 6027: (2S)-2-[(2S)-4-cyclohexyl-2-{[(2S)-1-(4-fluorobenzoyl) pyrrolidin-2-yl]formamido}butanamido]-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide or 8341: (2S)-2-[(2S)-4-cyclohexyl-2-{[(2S)-1-(4-fluorobenzoyl)pyrrolidin-2-yl]formamido}butanamido]-4-methyl-N-(1-methyl-1-{[(1S)-3-methyl-1-{[(1S)-3-methyl-1-{[1-methyl-1-({1-methyl-1-[(2-{[2-(morpholin-4-yl)ethyl]carbamoyl}ethyl)carbamoyl]ethyl}carbamoyl)ethyl]carbamoyl}butyl]carbamoyl}butyl]carbamoyl}ethyl)pentanamide.

In a further very preferred embodiment, said compound of formula (I) is 6025: (2S)-2-[(2S)-4-cyclohexyl-2-{[(2S)-1-(2-methyl-1,3-oxazole-4-carbonyl)pyrrolidin-2-yl]formamido}butanamido]-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide.

In a further very preferred embodiment, said compound of formula (I) is 6027: (2S)-2-[(2S)-4-cyclohexyl-2-{[(2S)-1-(4-fluorobenzoyl) pyrrolidin-2-yl]formamido}butanamido]-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide.

In a further very preferred embodiment, said compound of formula (I) is 8341: (2S)-2-[(2S)-4-cyclohexyl-2-{[(2S)-1-(4-fluorobenzoyl)pyrrolidin-2-yl]formamido}butanamido]-4-methyl-N-(1-methyl-1-{[(1S)-3-methyl-1-{[(1S)-3-methyl-1-{[1-methyl-1-({1-methyl-1-[(2-{[2-(morpholin-4-yl)ethyl]carbamoyl}ethyl)carbamoyl]ethyl}carbamoyl)ethyl]carbamoyl}butyl]carbamoyl}butyl]carbamoyl}ethyl)pentanamide.

In a further very preferred embodiment, said compound is selected from a formula as depicted in claim 10 and selected from 5469, 5768, 5769, 5770, 5904, 5905, 5906, 5910, 5911, 5912, 5934, 5936, 5937, 5938, 5939, 6025, 6026, 6027, 6028, 6253, 6254, 6328, 6483, 6488, 6779, 6780, 6789, 6791, 6894, 6896, 7050, 7058, 7059, 7192, 7842, 7846, 7848, 8341, 8342 or 8343 and wherein preferably said compound of formula (I) is selected from a formula as depicted in claim 10 and selected from 6025, 6027 or 8341. In a further very preferred embodiment, said compound of formula (I) is formula 6025 as depicted in claim 10. In a further very preferred embodiment, said compound of formula (I) is formula 6027 as depicted in claim 10. In a further very preferred embodiment, said compound of formula (I) is formula 8341 as depicted in claim 10.

In a further aspect, the present invention provides for a compound of formula (I), wherein said compound is selected from

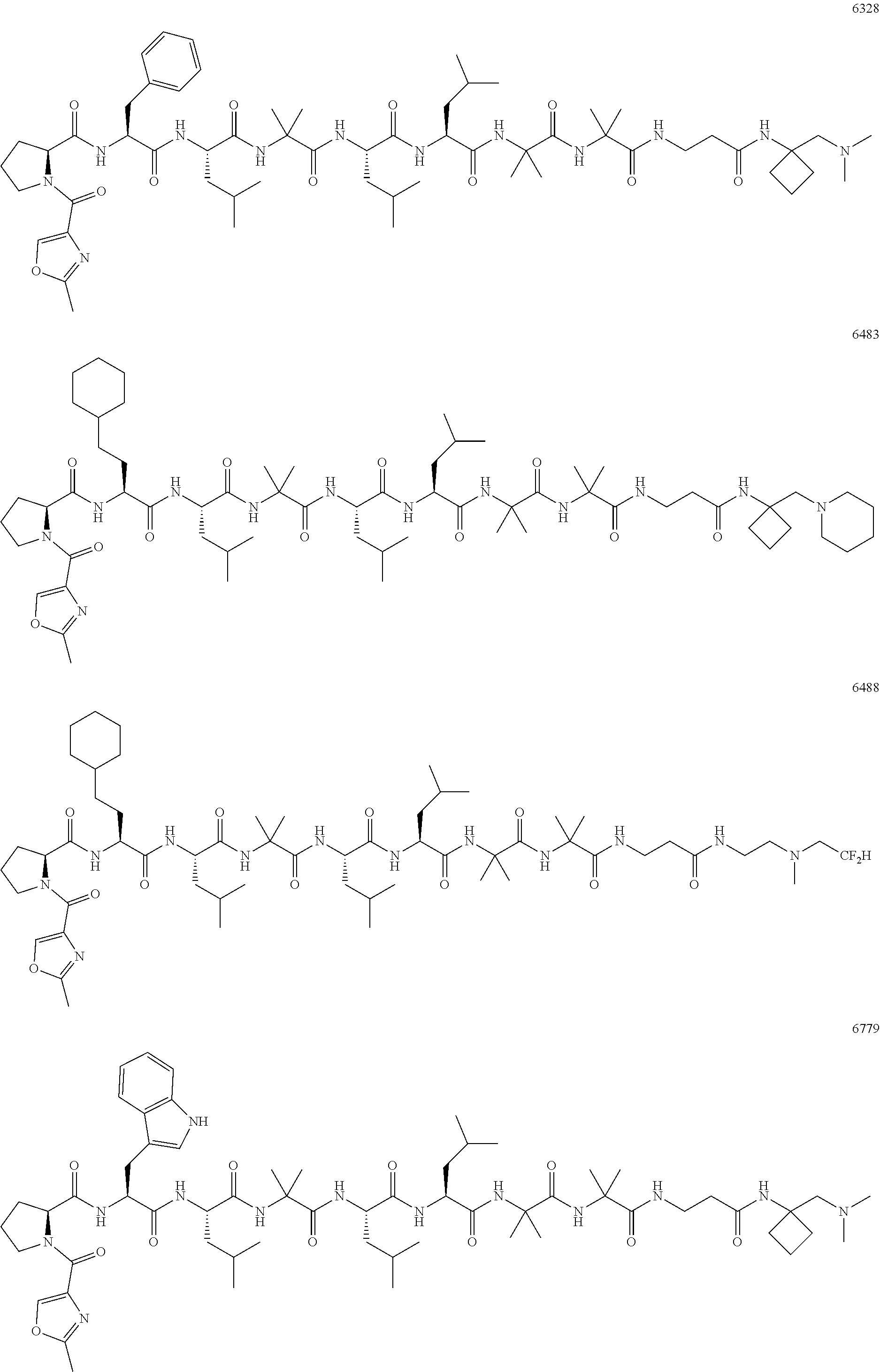

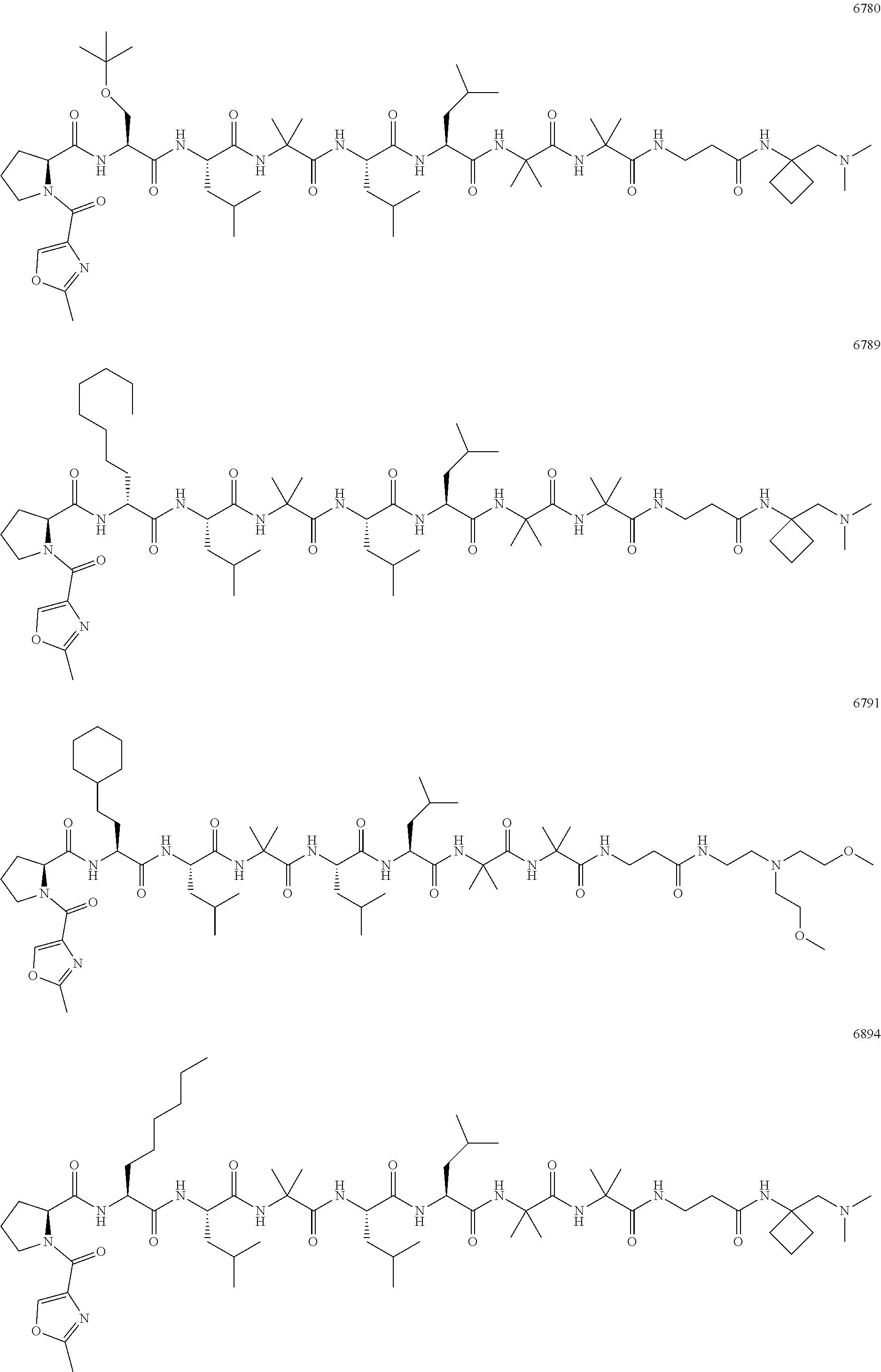

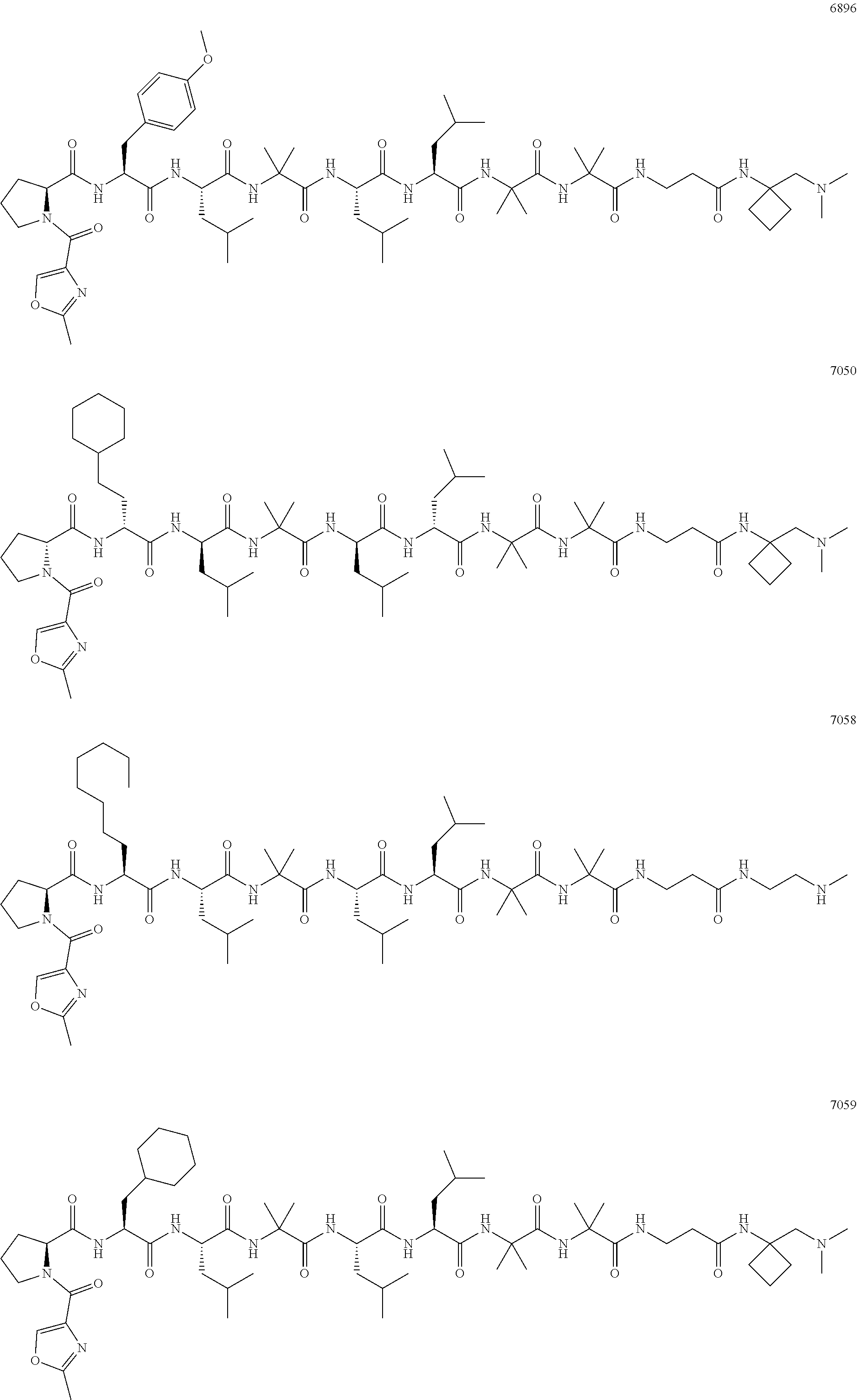

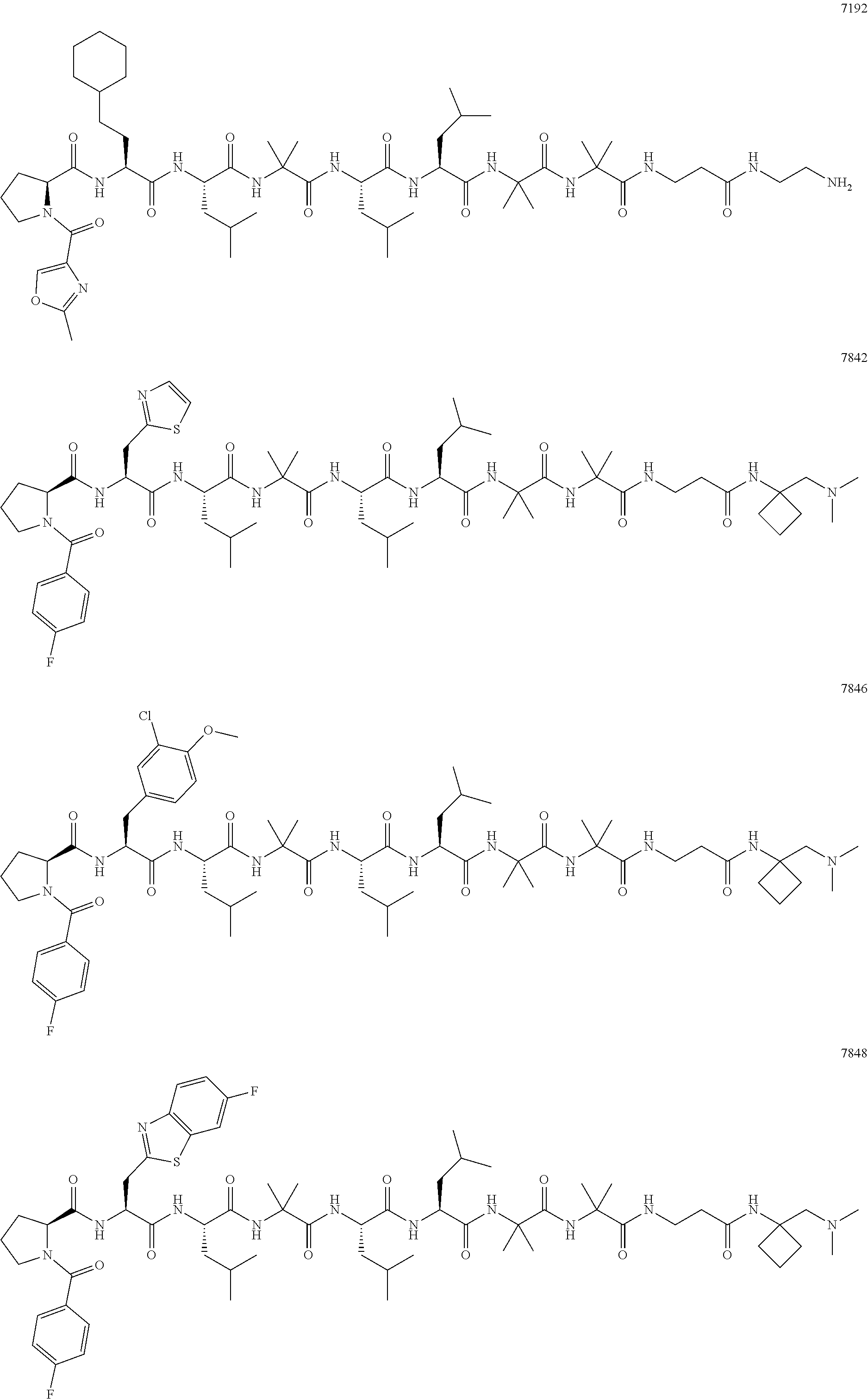

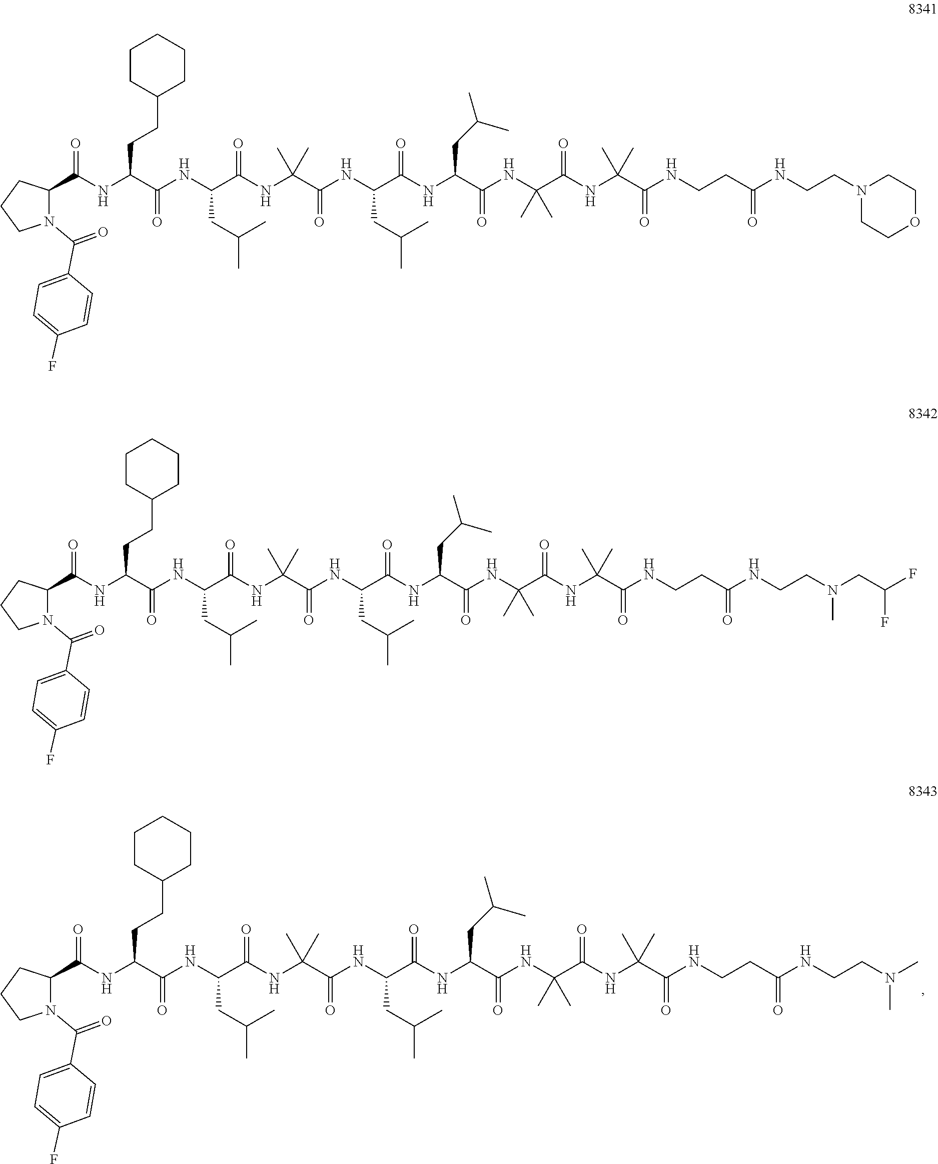

wherein preferably a compound of formula (I) is

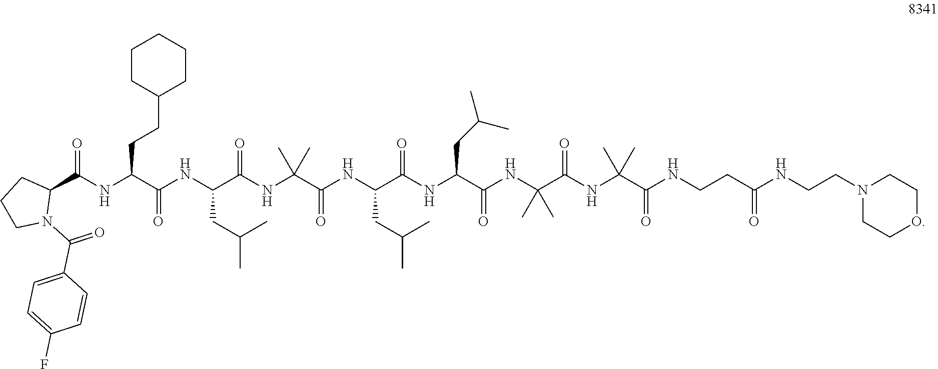 [SMILES: CC(C)C[C@H](NC(=O)[C@H](CCC1CCCCC1)NC(=O)[C@@H]1CCCN1C(=O)c1ccc(F)cc1)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NCCN1CCOCC1]

In a further aspect, the present invention provides for a compound of formula (I), wherein said compound is selected from
6328: (2S)-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methyl-2-[(2S)-2-{[(2S)-1-(2-methyl-1,3-oxazole-4-carbonyl)pyrrolidin-2-yl]formamido}-3-phenylpropanamido]pentanamide;
6483: (2S)-2-[(2S)-4-cyclohexyl-2-{[(2S)-1-(2-methyl-1,3-oxazole-4-carbonyl)pyrrolidin-2-yl]formamido}butanamido]-4-methyl-N-(1-methyl-1-{[(1S)-3-methyl-1-{[(1S)-3-methyl-1-({1-methyl-1-[(1-methyl-1-{[2-({1-[(piperidin-1-yl)methyl]cyclobutyl}carbamoyl)ethyl]carbamoyl}ethyl)carbamoyl]ethyl}carbamoyl)butyl]carbamyl}butyl]carbamoyl}ethyl)pentanamide,
6488: (2S)-2-[(2S)-4-cyclohexyl-2-{[(2S)-1-(2-methyl-1,3-oxazole-4-carbonyl)pyrrolidin-2-yl]formamido}butanamido]-N-(1-{[(1S)-1-{[(1S)-1-({1-[(1-{[2-({2-[(2,2-difluoroethyl)(methyl)amino]ethyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide;
6779: (2S)-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-2-[(2S)-3-(1H-indol-3-yl)-2-{[(2S)-1-(2-methyl-1,3-oxazole-4-carbonyl)pyrrolidin-2-yl]formamido}propanamido]-4-methylpentanamide;
6780: (2S)-2-[(2S)-2-{2-[(2S)-2-[(2S)-3-(tert-butoxy)-2-{[(2S)-1-(2-methyl-1,3-oxazole-4-carbonyl)pyrrolidin-2-yl]formamido}propanamido]-4-methylpentanamido]-2-methylpropanamido}-4-methylpentanamido]-N-{1-[(1-{[2-({1-[(dimethylamino)methyl]cyclobutyl}carbamoyl)ethyl]carbamoyl}-1-methylethyl)carbamoyl]-1-methylethyl}-4-methylpentanamide;
6789: (2R)-N-[(1S)-1-[(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)carbamoyl]-3-methylbutyl]-2-{[(2S)-1-(2-methyl-1,3-oxazole-4-carbonyl)pyrrolidin-2-yl]formamido}decanamide;
6791: (2S)-N-(1-{[(1S)-1-{[(1S)-1-({1-[(1-{[2-({2-[bis(2-methoxyethyl)amino]ethyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-2-[(2S)-4-cyclohexyl-2-{[(2S)-1-(2-methyl-1,3-oxazole-4-carbonyl)pyrrolidin-2-yl]formamido}butanamido]-4-methylpentanamide;
6894: (2S)—N-[(1S)-1-[(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)carbamoyl]-3-methylbutyl]-2-{[(2S)-1-(2-methyl-1,3-oxazole-4-carbonyl)pyrrolidin-2-yl]formamido}octanamide;
6896: (2S)-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-2-[(2S)-3-(4-methoxyphenyl)-2-{[(2S)-1-(2-methyl-1,3-oxazole-4-carbonyl)pyrrolidin-2-yl]formamido}propanamido]-4-methylpentanamide,
7050: (2R)-2-[(2R)-4-cyclohexyl-2-{[(2R)-1-(2-methyl-1,3-oxazole-4-carbonyl)pyrrolidin-2-yl]formamido}butanamido]-N-(1-{[(1R)-1-{[(1R)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide;
7058: (2S)-2-{[(2S)-1-(2-methyl-1,3-oxazole-4-carbonyl)pyrrolidin-2-yl]formamido}-N-[(1S)-3-methyl-1-[(1-methyl-1-{[(1S)-3-methyl-1-{[(1S)-3-methyl-1-{[1-methyl-1-({1-methyl-1-[(2-{[2-(methylamino)ethyl]carbamoyl}ethyl)carbamoyl]ethyl}carbamoyl) ethyl]carbamoyl}butyl]carbamoyl}butyl]carbamoyl}ethyl)carbamoyl]butyl]decanamide;
7059: (2S)-2-[(2S)-3-cyclohexyl-2-{[(2S)-1-(2-methyl-1,3-oxazole-4-carbonyl)pyrrolidin-2-yl]formamido}propanamido]-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide;
7192: (2S)-N-(1-{[(1S)-1-{[(1S)-1-[(1-{[1-({2-[(2-aminoethyl)carbamoyl]ethyl}carbamoyl)-1-methylethyl]carbamoyl}-1-methylethyl)carbamoyl]-3-methylbutyl]carbamoyl}-3-methylbutyl]carbamoyl}-1-methylethyl)-2-[(2S)-4-cyclohexyl-2-{[(2S)-1-(2-methyl-1,3-oxazole-4-carbonyl)pyrrolidin-2-yl]formamido}butanamido]-4-methylpentanamide;
7842: (2S)-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-2-[(2S)-2-{[(2S)-1-(4-fluorobenzoyl)pyrrolidin-2-yl]formamido}-3-(1,3-thiazol-2-yl)propanamido]-4-methylpentanamide;
7846: (2S)-2-[(2S)-3-(3-chloro-4-methoxyphenyl)-2-{[(2S)-1-(4-fluorobenzoyl)pyrrolidin-2-yl]formamido}propanamido]-N-(1-{[(1S)-1-{[(1S)-1-({1-[(1-1 [2-({1-[(dimethylamino)methyl]cyclobutyl 1 carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide;
7848: (2S)-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-2-[(2S)-3-(6-fluoro-1,3-benzothiazol-2-yl)-2-{[(2S)-1-(4-fluorobenzoyl)pyrrolidin-2-yl]formamido}propanamido]-4-methylpentanamide,
8341: (2S)-2-[(2S)-4-cyclohexyl-2-{[(2S)-1-(4-fluorobenzoyl)pyrrolidin-2-yl]formamido}butanamido]-4-methyl-N-(1-methyl-1-{[(1S)-3-methyl-1-{[(1S)-3-methyl-1-{[1-methyl-1-({1-methyl-1-[(2-{[2-(morpholin-4-yl)ethyl]carbamoyl}ethyl)carbamoyl]ethyl}carbamoyl)ethyl]carbamoyl}butyl]carbamoyl}butyl]carbamoyl}ethyl)pentanamide;
8342: (2S)-2-[(2S)-4-cyclohexyl-2-{[(2S)-1-(4-fluorobenzoyl)pyrrolidin-2-yl]formamido}butanamido]-N-(1-{[(1S)-1-{[(1S)-1-({1-[(1-{[2-({2-[(2,2-difluoroethyl)(methyl)amino]ethyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide;
8343: (2S)-2-[(2S)-4-cyclohexyl-2-{[(2S)-1-(4-fluorobenzoyl)pyrrolidin-2-yl]formamido}butanamido]-N-(1-{[(1S)-1-{[(1S)-1-{[1-({1-[(2-{[2-(dimethylamino)ethyl]carbamoyl}ethyl)carbamoyl]-1-methylethyl}carbamoyl)-1-methylethyl]carbamoyl}-3-methylbutyl]carbamoyl}-3-methylbutyl]carbamoyl}-1-methylethyl)-4-methylpentanamide;
   wherein preferably said compound is 8341: (2S)-2-[(2S)-4-cyclohexyl-2-{[(2S)-1-(4-fluorobenzoyl)pyrrolidin-2-yl]formamido}butanamido]-4-methyl-N-(1-methyl-1-{[(1S)-3-methyl-1-{[(1S)-3-methyl-1-{[1-methyl-1-({1-methyl-1-[(2-{[2-(morpholin-4-yl)ethyl]carbamoyl}ethyl)carbamoyl]ethyl}carbamoyl)ethyl]carbamoyl}butyl]carbamoyl}butyl]carbamoyl}ethyl)pentanamide.

The present invention provides a compound of formula (I) for use in a method of topical treatment of a skin disease or skin disorder of a mammal, wherein said method comprises topical administration of an effective amount of said compound to said mammal. In a preferred embodiment, said mammal is a human.

In a further preferred embodiment, said topical administration is applying said compound to a skin of a mammal. In a preferred embodiment, said topical administration is applying said compound to a skin of a human. In a further preferred embodiment, said topical administration is applying said compound directly to a skin of a mammal. In a preferred embodiment, said topical administration is applying said compound directly to a skin of a human.

In a further preferred embodiment, said skin disease or skin disorder is selected from a protozoan disease, a bacterial infection or a fungal infection. In a further preferred embodiment, said skin disease or skin disorder is a protozoan disease. In a further preferred embodiment, said skin disease or skin disorder is a bacterial infection. In a further preferred embodiment, said skin disease or skin disorder is a fungal infection.

In a further preferred embodiment, said skin disease or skin disorder is selected from a protozoan skin disease, a bacterial skin infection or a fungal skin infection. In a further preferred embodiment, said skin disease or skin disorder is a protozoan skin disease. In a further preferred embodiment, said skin disease or skin disorder is a bacterial skin infection. In a further preferred embodiment, said skin disease or skin disorder is a fungal skin infection.

In a further preferred embodiment, said skin disease or skin disorder is caused by a parasite, wherein said parasite is preferably a protozoan, by a fungus or by a bacteria. In a further preferred embodiment, said skin disease or skin disorder is caused by a parasite, preferably by a protozoan. In a further preferred embodiment, said skin disease or skin disorder is caused by a fungus. In a further preferred embodiment, said skin disease or skin disorder is caused by a bacteria.

In a further preferred embodiment, said skin disease or skin disorder is a protozoan disease. In a further preferred embodiment, said skin disease or skin disorder is a protozoan skin disease. In a further preferred embodiment, said skin disease or skin disorder is a protozoan skin infection. In a further preferred embodiment, said skin disease or skin disorder is caused by a parasite, preferably by a protozoan. In a further preferred embodiment, said skin disease or skin disorder is caused by a species of the genus Leishmania. In a further very preferred embodiment, said skin disease or skin disorder is cutaneous leishmaniasis.

The compounds and methods described herein are advantageously used to inhibit Leishmania parasites. Preferably, the compounds and methods provide topical treatment of a mammal, preferably a human, against a parasite in the Leishmania genus. The methods provides topical treatment against the various stages of Leishmania parasite infections, in particular various stages of infection with Leishmania genus and/or cutaneous forms of leishmaniasis. Thus, topical administering of a compound of formula (I) or a pharmaceutically acceptable salt thereof, or a pharmaceutical composition comprising the same provide treatment of a patient against Leishmania parasites, and in particular of a patient in a stage of infection with Leishmania genus against cutaneous forms of the disease. Topical administering of a compound of formula (I) or a pharmaceutically acceptable salt thereof, or pharmaceutical composition comprising the same to a patient leads to inhibiting Leishmania, by directly affecting the viability.

In a further preferred embodiment, said skin disease or skin disorder is a bacterial infection. In a further preferred embodiment, said skin disease or skin disorder is a bacterial skin infection. In a further preferred embodiment, said skin disease or skin disorder is caused by a bacteria. In a further preferred embodiment, said skin disease or skin disorder is caused by a gram-positive bacteria. In a further preferred embodiment, said skin disease or skin disorder is caused by a bacteria of genera Staphylococcus or Streptococcus. In a further preferred embodiment, said skin disease or skin disorder is caused by a bacteria of genera Staphylococcus. In a further preferred embodiment, said skin disease or skin disorder is caused by a bacteria of genera Streptococcus. In a further preferred embodiment, said skin disease or skin disorder is caused by a bacteria of genera Staphylococcus or Streptococcus selected from the group consisting of Staphylococcus aureus, Streptococcus pyogenes and Streptococcus agalactiae.

In a further preferred embodiment, said skin disease or skin disorder is caused by a bacteria of Staphylococcus aureus. Staphylococcus aureus (S. aureus) is the most common pathogen worldwide in hospital-acquired infections and is the cause of various infections of the skin and mucous membranes in children and adults. Although S. aureus usually acts as a commensal of the human microbiota it can also become an opportunistic pathogen, being a common cause of skin infections, which are the most common form of S. aureus infection. This can manifest in various ways, including small benign boils, folliculitis, impetigo, cellulitis, and more severe, invasive soft-tissue infections. It is mostly found in fertile, active places, including the armpits, hair, and scalp. S. aureus is an exceptionally adaptable bacterial species. The first resistant strains of S. aureus were already detected shortly after penicillin was introduced (1941) for treatment. Resistance to penicillinase-stable methicillin, which was introduced in 1959, was identified in 1960, after only one year of clinical use. Methicillin resistance (MRSA=methicillin-resistant S. aureus) now stands for resistance of S. aureus to (nearly) all beta-lactam antibiotics. Methicillin itself is no longer commercially available, although other isoxazolyl penicillins (e.g., flucloxacillin) are still used in clinical practice. Other, later, reported resistances include resistance to ciprofloxacin (1985), vancomycin (1998), and linezolid. For methicillin/oxacillin-sensitive S. aureus (MSSA) a selection of antibiotics are recommended under individual disease descriptions. For methicillin-/oxacillin-resistant S. aureus (MRSA) systemic topical antibiotics are recommended in combination with systemic antibiotics. Thus, in a further preferred embodiment, said skin disease or skin disorder is caused by multi-resistant Staphylococcus aureus (MRSA) strains, wherein preferably said multi-resistance is against one or more, preferably at least two, further preferably at least three, again further preferably at least four antibiotics selected from Methicillin, Oxacillin, Gentamicin, Levofloxacin, Vancomycin, Clindamycin, Erythromycin, and Linezolid.

In a further preferred embodiment, said skin disease or skin disorder is caused by a bacteria of Streptococcus pyogenes. Streptococcus pyogenes is the causative agent in a wide range of group A streptococcal infections (GAS). These infections may be non-invasive or invasive. The non-invasive infections tend to be more common and less severe. The most common of these infections include impetigo and streptococcal pharyngitis (strep throat). Certain strains have developed resistance to macrolides, tetracyclines, and clindamycin, making the development of new, potent drug candidates an urge. Streptococcus pyogenes is one of the most important bacterial causes of skin and soft tissue infections (SSTIs) worldwide. In addition, no other pathogen causes as many diverse clinical entities as S. pyogenes. Specifically, this organism causes infections including in the superficial keratin layer (impetigo), the superficial epidermis (erysipelas) and the subcutaneous tissue (cellulitis).

In a further preferred embodiment, said skin disease or skin disorder is caused by a bacteria of Streptococcus agalactiae. Streptococcus agalactiae, also known as Group B Streptococcus (GBS), was first differentiated from other streptococci by Rebecca Lancefield in the 1930s after it was isolated from milk and cows with bovine mastitis. Skin and soft tissue infections attributed to GBS may manifest as cellulitis, abscesses, foot infection, or decubitus ulcers. Diabetes mellitus is a common underlying condition in patients with GBS skin and soft tissue infections.

In a further preferred embodiment, said skin disease or skin disorder is a bacterial skin infection selected from carbuncles, ecthyma, erythrasma, folliculitis, furuncles, impetigo, lymphadenitis, skin abscesses, cellulitis, erysipelas, lymphangitis, necrotizing skin infections, staphylococcal scalded skin syndrome, wound infections.

In a further preferred embodiment, said skin disease or skin disorder is a fungal infection. In a further preferred embodiment, said skin disease or skin disorder is a fungal skin infection. In a further preferred embodiment, said skin disease or skin disorder is caused by a fungus. In a further preferred embodiment, said skin disease or skin disorder is caused by a fungus of the genera Trichophyton, Candida, Aspergillus, Cryptococcus, Histoplasma, Pneumocystis or Stachybotrys. In a further preferred embodiment, said skin disease or skin disorder is caused by a fungus of the genera Trichophyton or Candida. In a further preferred embodiment, said skin disease or skin disorder is caused by a fungus of the genera Trichophyton, preferably by Trichophyton mentagrophyte. In a further preferred embodiment, said skin disease or skin disorder is caused by a fungus of the genera Candida. In a further preferred embodiment, said a fungal infection is a tinea infection, wherein preferably said tinea infection is caused by Trichophyton mentagrophytes. In a further preferred embodiment, said skin disease is a tinea infection, wherein said tinea infection is caused by Trichophyton mentagrophytes.

### EXAMPLES

All reagents and solvents used in the synthesis were purchased from Sigma Aldrich, Bachem, Iris Biotech, Fluorochem, Enamine, Combi-Blocks and used as received. Solvents were stored over molecular sieves 4 Å. Peptides were synthesized on a 0.25-mmol or 0.10-mmol scale on Fmoc-β-Ala Wang resin (0.70 mmol g⁻¹) using a Liberty Blue microwave peptide synthesizer (CEM Corp., Matthews, NC) employing Fmoc solid-phase techniques with repeated steps of coupling, deprotection, and washing. Coupling was performed as follows: Fmoc-L-amino acids or the capping group (5.0 equiv., 0.2 M in DMF), DIC (5.0 equiv., 0.5 M in DMF), and Oxyma (5.0 equiv., 1.0 M in DMF) for 4 minutes with microwave irradiation at 90° C. For the second AIB coupling, a double coupling with each 15 minutes microwave irradiation at 90° C. was performed. Fmoc deprotection was performed as follows: 10% piperazine in NMP/ethanol (9:1) for 1 minute with microwave irradiation at 90° C. Following synthesis, the peptide was cleaved from the resin by treatment with a cleavage mixture (1 mL/0.1 g resin) consisting of TFA/H2O (95:5) for 90 minutes at ambient temperature. The suspended resin was removed by filtration and concentrated in vacuo. The crude peptides were dissolved in acetonitrile and were purified by ISCO chromatography system using as mobile phase H₂O/acetonitrile (95:5, v/v) and acetonitrile/H₂O, gradient ACN 10% to 100%, to give peptide acids ≥90% pure. The purified peptide acid (1.0 equiv.) was coupled in solution with suitable primary or secondary amines (2.0 equiv.) in presence of DIPEA (3.0 equiv.) and COMU (2.0 equiv.) to give the final peptide. Solvent was partially removed, and the crude peptide was directly purified by Gilson PLC 2020 Personal Purification System, as described above, to give final products >95% pure.

High-resolution mass spectrometry was performed on an Agilent Technologies 6530 Q-TOF. Mass and purity of final compounds was determined by UPLC-MS using a Waters Acquity system equipped with a Waters BEH C18 column and diode array detector (254 nm). The mobile phase consisted of H₂O-Acetonitrile (solvent A, 97:3 v/v, LC-MS Ultra Chromasolv®, UHPLC grade, Sigma-Aldrich, Germany) and 0.1% Formic acid (LC-MS grade, Sigma-Aldrich, Germany) and Acetonitrile-H₂O (solvent B, 97:3 v/v, LC-MS Ultra Chromasolv®, UHPLC grade, Sigma-Aldrich, Germany) and 0.1% Formic acid (LC-MS grade, Sigma-Aldrich, Germany) with a 4 min run time, 0.6 ml/min flow rate, 10 μL injection volume and a gradient elution according to the following program: linear increment starting with 100% A to 100% Bin 3 min and returning to the initial conditions within the next 1 min. MS detection of analytes was performed on a Waters Xevo® triple quadrupole mass spectrometer equipped with electrospray ionization (ESI) interface (Waters, Xevo® TQD QCA065) in positive and negative ion mode with mass range from 100-1500 m/z.

### Example 1

### General Procedure for the Synthesis for Compounds of Formula (I)

The peptides were synthesized using a Liberty Blue microwave peptide synthesizer (CEM Corp., Matthews, NC) starting from Fmoc-beta-Ala linked to Wang resin as depicted in Scheme 1, employing Fmoc solid-phase techniques with repeated steps of coupling, deprotection, and washing. Coupling was performed as follows: Fmoc-L-amino acids or the capping group (5.0 equiv., 0.2 M in DMF), DIC (5.0 equiv., 0.5 M in DMF), and Oxyma (5.0 equiv., 1.0 M in DMF) for 4 minutes with microwave irradiation at 90° C. For the second Aib coupling, a double coupling with each 15 minutes microwave irradiation at 90° C. was performed. Fmoc deprotection was performed as follows: 10% piperazine in NMP/ethanol (9:1) for 1 minute with microwave irradiation at 90° C. By employing sequentially the building blocks listed in Table 1 (Amino acids used in steps 1 to 8, Scheme 1) and free acid derivatives (step 9, Scheme 1) listed in Table 2, the different peptides were assembled on solid phase support in typically and preferably 9 steps. Following synthesis, the peptide was cleaved from the resin by treatment with a cleavage mixture (1 mL/0.1 g resin) consisting of TFA/H₂O (95:5) for 90 minutes at ambient temperature. The suspended resin was removed by filtration and concentrated in vacuo. The crude peptides were dissolved in acetonitrile and were purified by ISCO chromatography system. The purified peptide acid was coupled in solution with suitable primary or secondary amines reported in Table 3 (step 10, Scheme 1) in presence of DIPEA and COMU to give the final peptide, which was then directly purified on Gilson PLC 2020 Personal Purification System.

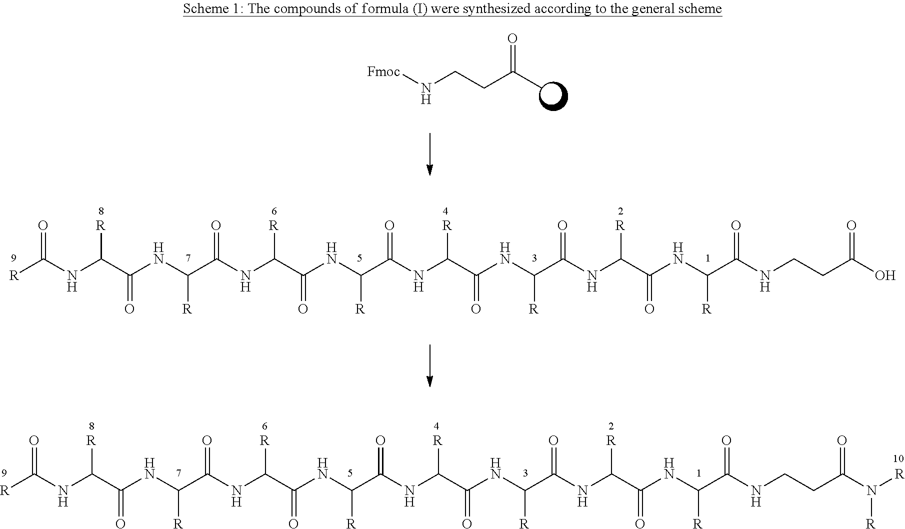

TABLE 1
Amino acid building blocks used for the synthesis of compounds of formula (I)
AMINO ACIDS

| | Structure | IUPAC Name |
|---|---|---|
| Fmoc-Aib-OH | 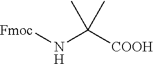 | 2-((((9H-fluoren-9-yl)methoxy) carbonyl)amino)-2- methylpropanoic acid |
| Fmoc-Leu-OH | 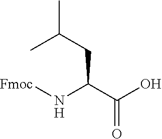 | (((9H-fluoren-9-yl)methoxy) carbonyl)-L-leucine |
| Fmoc-D-Leu-OH | 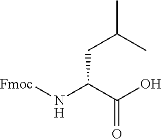 | (((9H-fluoren-9-yl)methoxy) carbonyl)-D-leucine |
| Fmoc-(S)-2-amino- decanoic acid | 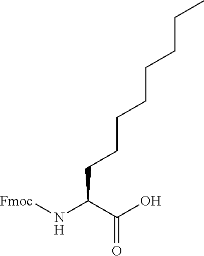 | (S)-2-((((9H-fluoren-9-yl) methoxy)carbonyl)amino) decanoic acid |
| Fmoc-Pro-OH | 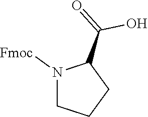 | (((9H-fluoren-9-yl)methoxy) carbonyl)-L-proline |
| Fmoc-D-Pro-OH | 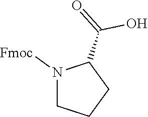 | (((9H-fluoren-9-yl)methoxy) carbonyl)-D-proline |
| Fmoc-L- homocyclohexylalanine | 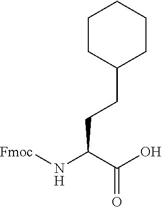 | (S)-2-((((9H-fluoren-9-yl) methoxy)carbonyl)amino)-4- cyclohexylbutanoic acid |
| Fmoc-D- homocyclohexylalanine | 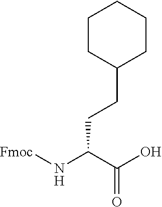 | (R)-2-((((9H-fluoren-9-yl) methoxy)carbonyl)amino)-4- cyclohexylbutanoic acid |
| Fmoc-(R)-2-amino- decanoic acid | 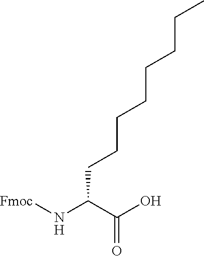 | (R)-2-((((9H-fluoren-9-yl) methoxy)carbonyl)amino) decanoic acid |
| Fmoc-(S)-2- aminooctanoic acid | 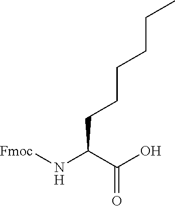 | Fmoc-(S)-2-aminooctanoic acid |
| Fmoc-Ser(tBu)-OH | 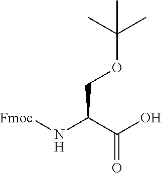 | N-(((9H-fluoren-9-yl)methoxy) carbonyl)-0-(tert-butyl)-L-serine |
| Fmoc-His-OH | 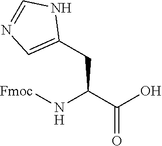 | (S)-2-((((9H-Fluoren-9-yl) methoxy)carbonyl)amino)-3-(1H- imidazol-4-yl)propanoic acid |
| Fmoc-Phe-OH | 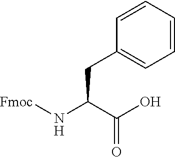 | (2S)-2-(9H-fluoren-9-yl methoxycarbonylamino)-3- phenyl-propanoic acid |
| Fmoc-Trp-OH | 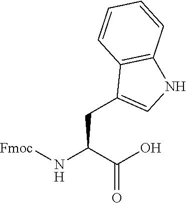 | (S)-2-(9H-Fluoren-9-yl methoxycarbonylamino)-3-(1H- indol-3-yl)-propionic acid |
| Fmoc-Cha-OH | 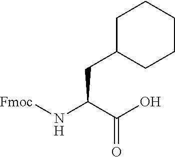 | (S)-2-((((9H-Fluoren-9-yl) methoxy)carbonyl)amino)-3- cyclohexylpropanoic acid |
| Fmoc-Tyr(Me)-OH | 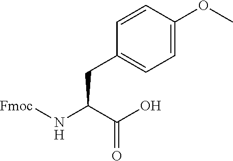 | (S)-2-((((9H-Fluoren-9-yl) methoxy)carbonyl)amino)-3-(4- methoxyphenyl)propanoic acid |
| (2S)-2-({[(9H-fluoren-9- yl)methoxy]carbonyl} amino)-3-(6-fluoro-1,3- benzothiazol-2-yl) propanoic acid | 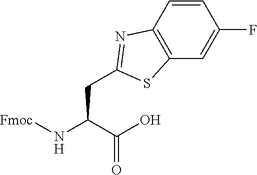 | (2S)-2-({[(9H-fluoren-9-yl) methoxy]carbonyl}amino)-3-(6- fluoro-1,3-benzothiazol-2-yl) propanoic acid |
| (2S)-3-(3-chloro-4- methoxyphenyl)-2-({[(9H- fluoren-9-yl)methoxy] carbonyl}amino) propanoic acid | 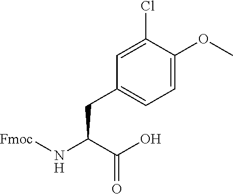 | (2S)-3-(3-chloro-4-methoxy- phenyl)-2-({[(9H-fluoren-9-yl) methoxy]carbonyl}amino) propanoic acid |

TABLE 2
Free acids used for the synthesis of compounds of formula (I)
(step 9, Scheme 1)
ACIDS

| Short Name | Structure | IUPAC Name |
|---|---|---|
| 2-methyloxazole-4- carboxylic acid | 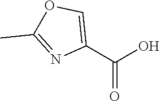 | 2-methyloxazole-4- carboxylic acid |
| 4-Fluorobenzoic acid | 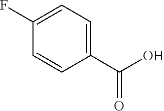 | 4-Fluorobenzoic acid |

TABLE 3
Primary or secondary amines used for the synthesis of compounds of formula (I) (step
10, Scheme 1).
AMINES

| Short Name | Structure | IUPAC Name |
|---|---|---|
| [(2S)-2-aminopropyl] dimethylamine | 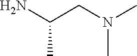 | [(2S)-2- aminopropyl]dimethylamine |
| 1-[(dimethylamino)methyl] cyclobutan-1-amine | 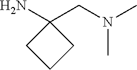 | 1-[(dimethylamino)methyl] cyclobutan-1-amine |
| (2-aminoethyl)dimethylamine | 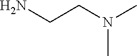 | (2-aminoethyl)dimethylamine |
| (2-aminoethyl)bis(2- methoxyethyl)amine dihydrochloride | 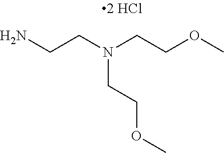 | (2-aminoethyl)bis(2- methoxyethyl)amine dihydrochloride |
| 1-(piperidin-1-ylmethyl) cyclobutan-1-amine dihydrochloride | 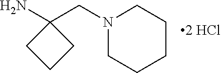 [SMILES: NC1(CN2CCCCC2)CCC1] | 1-(piperidin-1-ylmethyl) cyclobutan-1-amine dihydrochloride |
| 2-(morpholin-4-yl)ethan-1- amine | 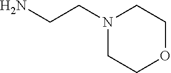 | 2-(morpholin-4-yl)ethan-1- amine |
| (2,2-difluoro-ethyl)-methyl- amine | 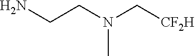 | (2,2-difluoro-ethyl)-methyl- amine |

### Example 2

### Synthesis of Amino Acids as Building Blocks for Compounds of Formula (I)

Some amino acids needed for the synthesis of compounds of formula (I) were not commercially available. In this case, they were synthesized. In Scheme 2 these syntheses are presented.

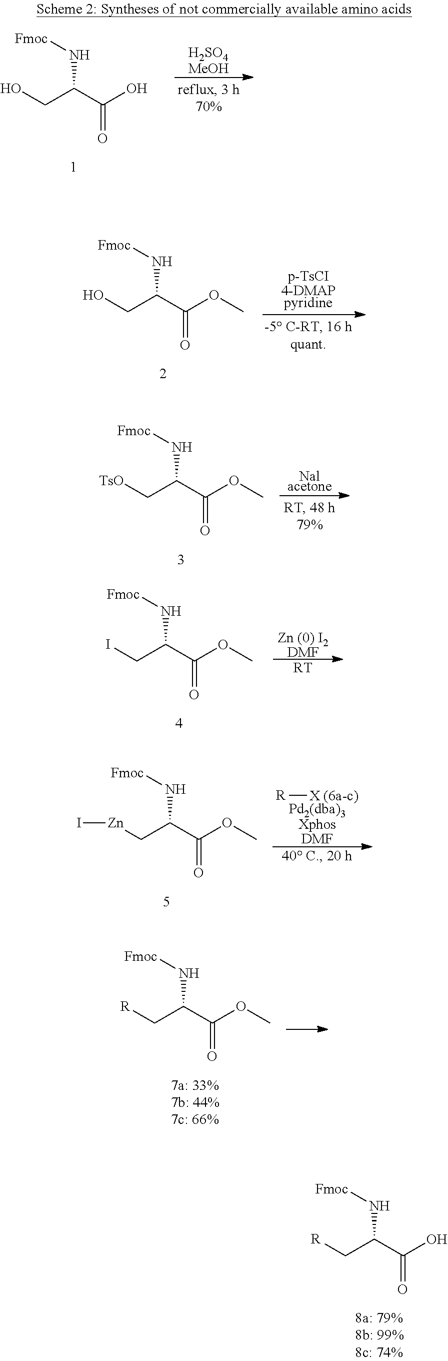

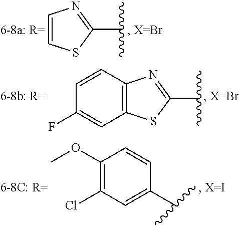

The synthesis of the amino acids 8a-c is depicted at Scheme 2. To begin with, FMOC-Ser-OH (1, 30.6 mmol, 1.0 equiv.) was suspended in MeOH (6 ml/mmol of 1) and drops of conc. H₂SO₄ were added. The reaction mixture was set under reflux for 3 h. Then it was cooled down to RT, the pH was adjusted to 8.0 using an aq. 20% w/v Na₂CO₃ sol. and extracted with EtOAc (3×150 ml). The organic layer was washed with brine, dried over MgSO₄, and evaporated to give 2 as a white solid.

In the next step, 2 (1.0 equiv.) and 4-DMAP (0.12 equiv.) were dissolved in dry pyridine (0.6 ml/mmol of 2) and cooled to −5° C. p-TsOH (3.3 equiv.) was added to the solution and the resultant yellow reaction mixture was stirred under nitrogen for 16 h. Then, the reaction was poured into ice-water and the aqueous mixture was extracted into ethyl acetate (3×100 mL). The combined organic layer was washed with water (2×100 ml), 10% w/v KHSO₄ solution (2×100 mL), sat. NaHCO₃ sol. (2×100 mL) and brine (2×100 ml). The organic layers were dried over Na₂SO₄, filtered, and concentrated under reduced pressure to give the tosylate 3. Following, a solution of sodium iodide (5.0 equiv.) in acetone (0.0 mL/mmol of NaI) was added dropwise to a solution of the tosylate 3 (1.0 equiv.) in acetone (1.0 mL/mmol of 3) under nitrogen. The resulting yellow solution was stirred for 48 h at RT. The reaction mixture was filtered, and the filtrate was concentrated under reduced pressure. The residue was dissolved in chloroform (100 mL), washed with water (2×50 mL), sat. Na₂S₂O₃ sol. (2×50 mL) and brine (2×30 mL). The organic layer was dried upon Na₂SO₄, filtered and concentrated under reduced pressure to give 4 a pale-yellow solid. Afterwards, Zn (0) (dust) (4.0 equiv.) was weighted into a flame-dried flask and 12 (0.22 equiv.) dissolved in dry DMF (1.0 ml/mmol of Zn(0)) was added. Iodine 4 (1.0 equiv.) together with 12 was added and the reaction mixture was stirred until full consumption (TLC indicated).

Most of the supernatant was taken out in a syringe and directly added to a pre-stirred solution of Pd₂(dba)₃ (0.05 mml), Xphos (0.10 mmol) and the respective halide 6a-c (1.3 mmol) in dry DMF (1.0 ml/mmol of Zn(0)). The reaction mixtures were stirred for 2 h at 40° C. Then the mixture was poured into sat. NaHCO₃ sol (10 ml) and extracted with EtOAc (2×10 ml). The combined organic layers were washed with sat. NaCl sol., dried over MgSO₄, concentrated, and dried under vacuum. The crude product was purified via flash column chromatography to furnish the esters 7a-c. Finally, to a suspension of 7a-c (1.0 equiv.) in 0.8 M CaCl₂ solution in ⁱPrOH/H₂O (20 ml/mmol of 7), NaOH (1.2 equiv.) was added and the mixture was stirred at RT overnight. The reaction was quenched by the addition of acetic acid and the solvent was removed under reduced pressure. The final amino acids 8a-c were obtained by the means of RP chromatography of the crude products.

### Example 3

### Synthesis of Compounds of Formula (I)

The synthesis of compounds of formula (I) have already described in EP17150353 including the synthesis of compounds 5649, 5768, 5769, 5770, 5904, 5905, 5906, 5910, 5911, 5912, 5934, 5936, 5937, 5938, 5939, 6025, 6026, 6027, 6028, 6253, 6254. In an analogous manner, except if mentioned otherwise, the compounds of formula (I) further described thereafter have been synthesized according to the general procedure depicted in Scheme 1. Each of the synthesized compounds are identified by its compound number (4-digit number), its structural formula and its IUPAC name as generated by the MarvinSketch software. Furthermore, the synthesis of 6328, 6483, 6488, 6779, 6780, 6789, 6791, 6894, 6896, 7050, 7058, 7059, 7192, 7842, 7846, 7848, 8341, 8342 and 8343 is described by way of the building blocks, acids and amines used as disclosed in Tables 1-3. Moreover, the experimentally measured molecular mass is provided. Finally, for each specifically identified compound of formula (I) the characterization of the substituents R₁, R₂, and R₃ in accordance with formula (I) is given, wherein the residue R within said definition of said substituents R₁, R₂, and R₃ corresponds to and indicates the respective attachment within formula (I).

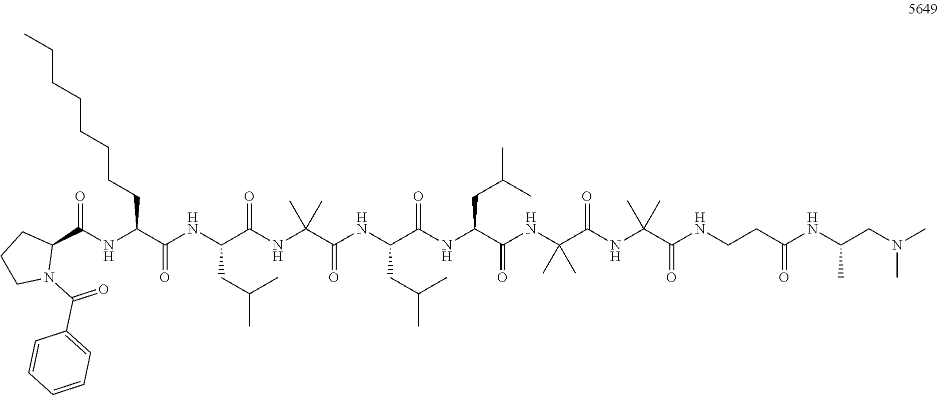 [SMILES: CCCCCCCC[C@H](NC(=O)[C@@H]1CCCN1C(=O)c1ccccc1)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)N[C@@H](C)CN(C)C]

(2S)-2-{[(2S)-1-benzoylpyrrolidin-2-yl]formamido}-N-[(1S)-1-[(1-{[(1S)-1-{[(1S)-1-{[1-({1-[(2-{[(2S)-1-(dimethylamino)propan-2-yl]carbamoyl}ethyl)carbamoyl]-1-methylethyl}carbamoyl)-1-methylethyl]carbamoyl}-3-methylbutyl]carbamoyl}-3-methylbutyl]carbamoyl}-1-methylethyl)carbamoyl]-3-methylbutyl]decanamide.

| Cpd | R1 | R2 | R3 |
|---|---|---|---|
| 5649 | 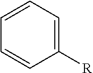 | 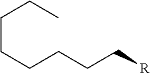 | 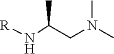 |

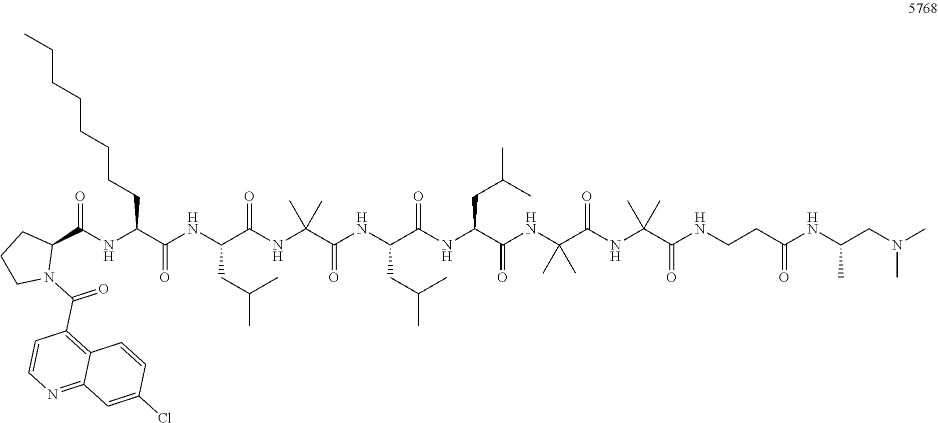 [SMILES: CCCCCCCC[C@H](NC(=O)[C@@H]1CCCN1C(=O)c1ccnc2cc(Cl)ccc12)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)N[C@@H](C)CN(C)C]

(2S)-2-{[(2S)-1-(7-chloroquinoline-4-carbonyl)pyrrolidin-2-yl]formamido}-N-[(1S)-1-[(1-{[(1S)-1-{[(1S)-1-{[1-({1-[(2-{[(2S)-1-(dimethylamino)propan-2-yl]carbamoyl}ethyl)carbamoyl]-1-methylethyl}carbamoyl)-1-methylethyl]carbamoyl}-3-methylbutyl]carbamoyl}-3-methylbutyl]carbamoyl}-1-methylethyl)carbamoyl]-3-methylbutyl]decanamide

| Cpd | R1 | R2 | R3 |
|---|---|---|---|
| 5768 | 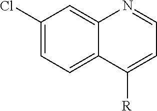 | 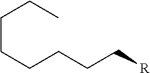 | 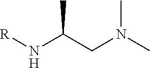 |

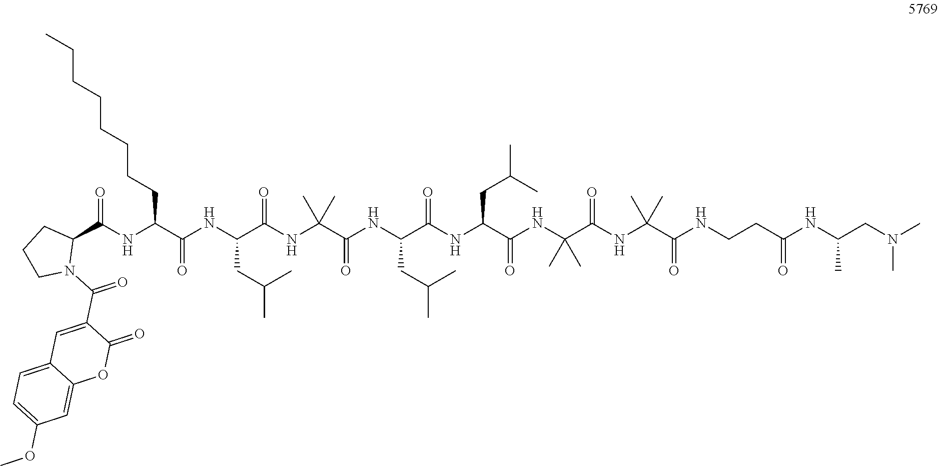

(2S)-N-[(1S)-1-[(1-{[(1S)-1-{[(1S)-1-{[1-({1-[(2-{[(2S)-1-(dimethylamino)propan-2-yl]carbamoyl}ethyl)carbamoyl]-1-methylethyl}carbamoyl)-1-methylethyl]carbamoyl}-3-methylbutyl]carbamoyl}-3-methylbutyl]carbamoyl}-1-methylethyl)carbamoyl]-3-methylbutyl]-2-{[(2S)-1-(7-methoxy-2-oxo-2H-chromene-3-carbonyl)pyrrolidin-2-yl]formamido}decanamide.

| Cpd | R1 | R2 | R3 |
|---|---|---|---|
| 5769 | 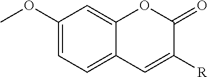 | 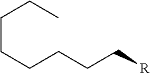 | 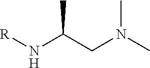 |

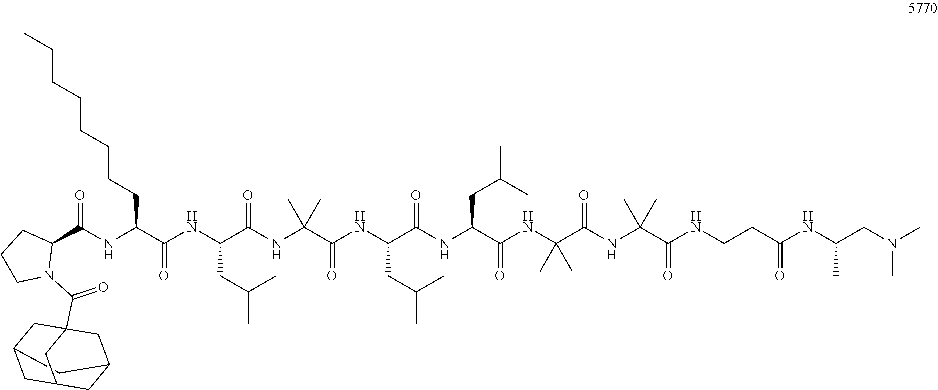 [SMILES: CCCCCCCC[C@H](NC(=O)[C@@H]1CCCN1C(=O)C12CC3CC(CC(C3)C1)C2)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)N[C@@H](C)CN(C)C]

(2S)-2-{[(2S)-1-(adamantane-1-carbonyl)pyrrolidin-2-yl]formamido}-N-[(1S)-1-[(1-{[(1S)-1-{[(1S)-1-{[1-({1-[(2-{[(2S)-1-(dimethylamino)propan-2-yl]carbamoyl}1 ethyl)carbamoyl]-1-methylethyl}carbamoyl)-1-methylethyl]carbamoyl}-3-methylbutyl]carbamoyl}-3-methylbutyl]carbamoyl}-1-methylethyl)carbamoyl]-3-methylbutyl]decanamide.

| Cpd | R1 | R2 | R3 |
|---|---|---|---|
| 5770 | 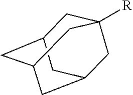 | 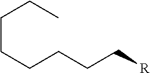 | 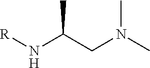 |

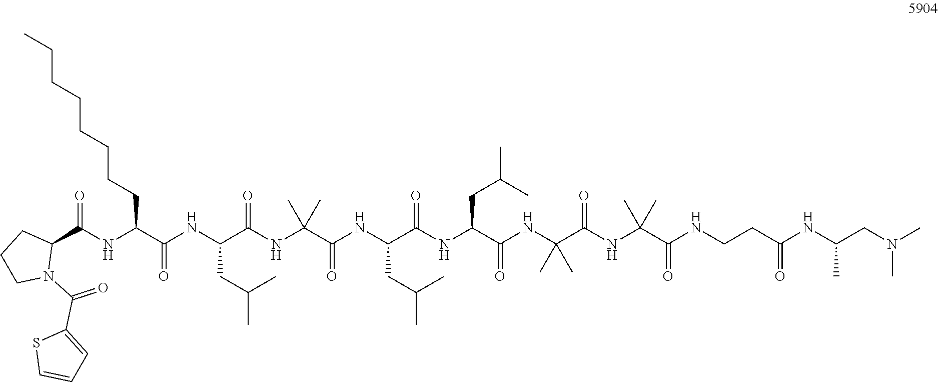 [SMILES: CCCCCCCC[C@H](NC(=O)[C@@H]1CCCN1C(=O)c1cccs1)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)N[C@@H](C)CN(C)C]

(2S)-N-[(1S)-1-{[(1-{[(1S)-1-[(1S)-1-{[1-({1-[(2-{[(2S)-1-(dimethylamino)propan-2-yl]carbamoyl}ethyl)carbamoyl]-1-methylethyl}carbamoyl)-1-methylethyl]carbamoyl}-3-methylbutyl]carbamoyl}-3-methylbutyl]carbamoyl}-1-methylethyl)carbamoyl]-3-methylbutyl]-2-{[(2S)-1-(thiophene-2-carbonyl)pyrrolidin-2-yl]formamido}decanamide.

| Cpd | R1 | R2 | R3 |
|---|---|---|---|
| 5904 | 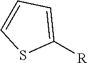 | 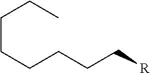 | 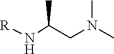 |

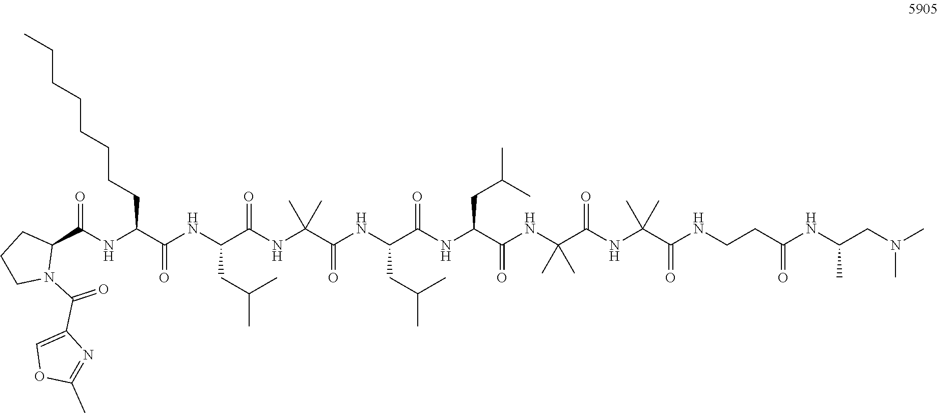 [SMILES: CCCCCCCC[C@H](NC(=O)[C@@H]1CCCN1C(=O)c1coc(C)n1)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)N[C@@H](C)CN(C)C]

(2S)-N-[(1S)-1-[(1-{[(1S)-1-{[(1S)-1-{[1-({1-[(2-{[(2S)-1-(dimethylamino)propan-2-yl]carbamoyl}ethyl)carbamoyl]-1-methylethyl}carbamoyl)-1-methylethyl]carbamoyl}-3-methylbutyl]carbamoyl}-3-methylbutyl]carbamoyl}-1-methylethyl)carbamoyl]-3-methylbutyl]-2-{[(2S)-1-(2-methyl-1,3-oxazole-4-carbonyl)pyrrolidin-2-yl]formamido}decanamide.

| Cpd | R1 | R2 | R3 |
|---|---|---|---|
| 5905 | 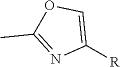 | 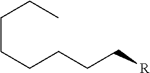 | 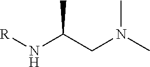 |

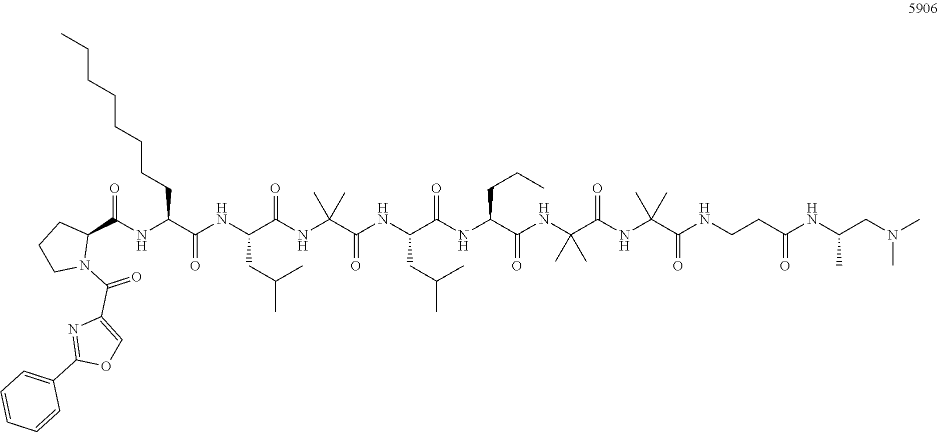 [SMILES: CCCCCCCC[C@H](NC(=O)[C@@H]1CCCN1C(=O)c1coc(-c2ccccc2)n1)C(=O)N[C@@H](CC(C)C)C(=O)NC(C)(C)C(=O)N[C@@H](CC(C)C)C(=O)N[C@@H](CCC)C(=O)NC(C)(C)C(=O)NC(C)(C)C(=O)NCCC(=O)N[C@@H](C)CN(C)C]

(2S)-N-[(1S)-1-[(1-{[(1S)-1-{[(1S)-1-{[1-({1-[(2-{[(2S)-1-(dimethylamino)propan-2-yl]carbamoyl}ethyl)carbamoyl]-1-methylethyl}carbamoyl)-1-methylethyl]carbamoyl}-3-methylbutyl]carbamoyl}-3-methylbutyl]carbamoyl}-1-methylethyl)carbamoyl]-3-methylbutyl]-2-{[(2S)-1-(2-phenyl-1,3-oxazole-4-carbonyl)pyrrolidin-2-yl]formamido}decanamide.

| Cpd | R1 | R2 | R3 |
|---|---|---|---|
| 5906 | 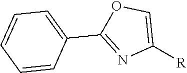 | 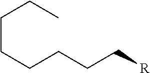 | 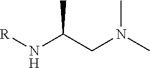 |

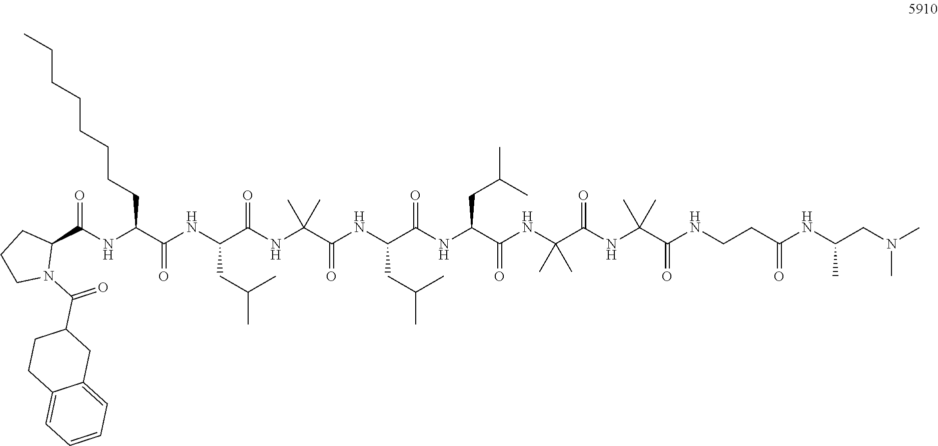 [SMILES: CCCCCCCC[C@H](NC(=O)[C@@H]1CCCN1C(=O)C1CCc2ccccc2C1)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)N[C@@H](C)CN(C)C]

(2S)-N-[(1S)-1-[(1-{[(1S)-1-{[(1S)-1-{[1-({1[(2-{[(2S)-1-(dimethylamino)propan-2-yl]carbamoyl}ethyl)carbamoyl]-1-methylethyl}carbamoyl)-1-methylethyl]carbamoyl}-3-methylbutyl]carbamoyl}-3-methylbutyl]carbamoyl}-1-methylethyl)carbamoyl]-3-methylbutyl]-2-{[(2S)-1-(1,2,3,4-tetrahydronaphthalene-2-carbonyl)pyrrolidin-2-yl]formamido}decanamide.

| Cpd | R1 | R2 | R3 |
|---|---|---|---|
| 5910 | 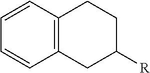 | 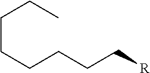 | 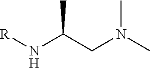 |

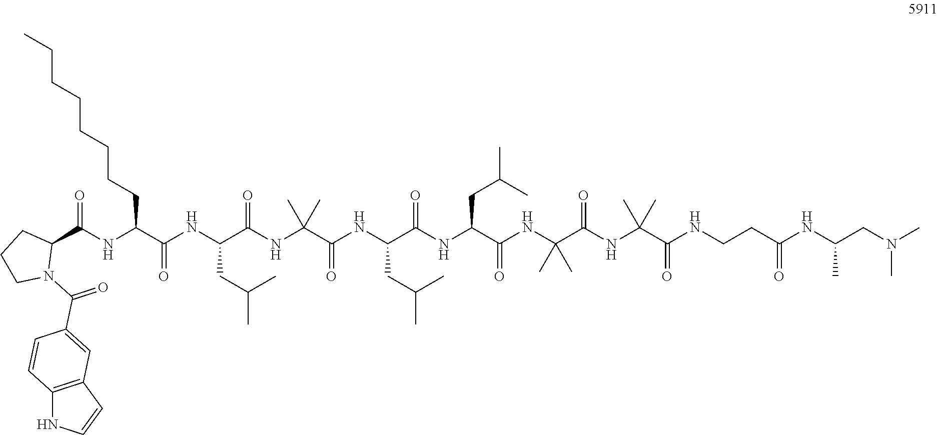 [SMILES: CCCCCCCC[C@H](NC(=O)[C@@H]1CCCN1C(=O)c1ccc2[nH]ccc2c1)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)N[C@@H](C)CN(C)C]

(2S)-N-[(1S)-1-[(1-{[(1S)-1-{[(1S)-1-{[1-({1-[(2-{[(2S)-1-(dimethylamino)propan-2-yl]carbamoyl}ethyl)carbamoyl]-1-methylethyl}carbamoyl)-1-methylethyl]carbamoyl}-3-methylbutyl]carbamoyl}-3-methylbutyl]carbamoyl}-1-methylethyl)carbamoyl]-3-methylbutyl]-2-{[(2S)-1-(1H-indole-5-carbonyl)pyrrolidin-2-yl]formamido}decanamide.

| Cpd | R1 | R2 | R3 |
|---|---|---|---|
| 5911 | 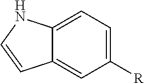 | 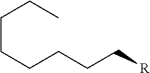 | 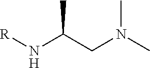 |

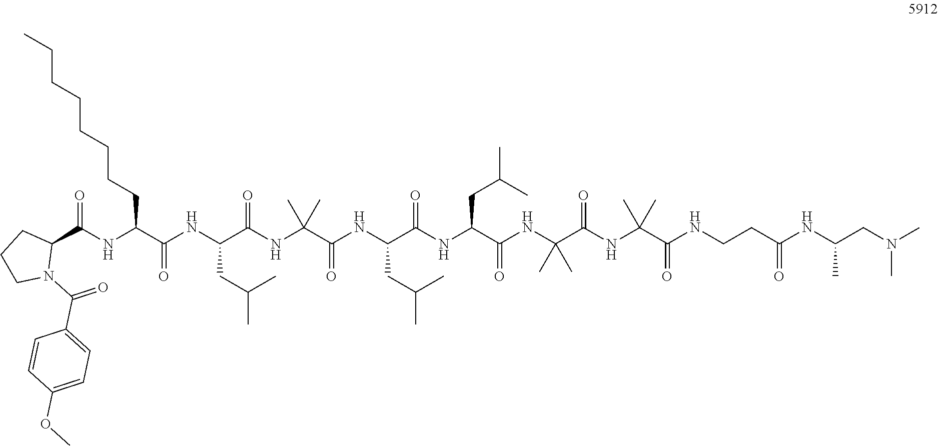 [SMILES: CCCCCCCC[C@H](NC(=O)[C@@H]1CCCN1C(=O)c1ccc(OC)cc1)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)N[C@@H](C)CN(C)C]

(2S)-N-[(1S)-1-[(1-{[(1S)-1-{[(1S)-1-{[1-({1-[(2-{[(2S)-1-(dimethylamino)propan-2-yl]carbamoyl}ethyl)carbamoyl]-1-methylethyl}carbamoyl)-1-methylethyl]carbamoyl}-3-methylbutyl]carbamoyl}-3-methylbutyl]carbamoyl}-1-methylethyl)carbamoyl]-3-methylbutyl]-2-{[(2S)-1-(4-methoxybenzoyl)pyrrolidin-2-yl]formamido}decanamide.

| Cpd | R1 | R2 | R3 |
|---|---|---|---|
| 5912 | 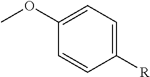 | 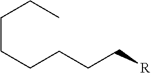 | 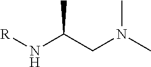 |

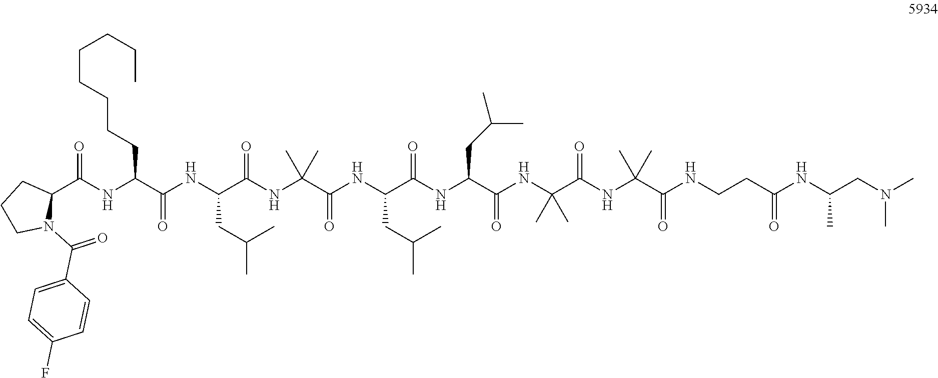 [SMILES: CCCCCCCC[C@H](NC(=O)[C@@H]1CCCN1C(=O)c1ccc(F)cc1)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)N[C@@H](C)CN(C)C]

(2S)-N-[(1S)-1-[(1-{[(1S)-1-{[(1S)-1-{[1-({1-[(2-{[(2S)-1-(dimethylamino)propan-2-yl]carbamoyl}ethyl)carbamoyl]-1-methylethyl}carbamoyl)-1-methylethyl]carbamoyl}-3-methylbutyl]carbamoyl}-3-methylbutyl]carbamoyl}-1-methylethyl)carbamoyl]-3-methylbutyl]-2-{[(2S)-1-(4-fluorobenzoyl)pyrrolidin-2-yl]formamido}decanamide

| Cpd | R1 | R2 | R3 |
|---|---|---|---|
| 5934 | 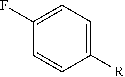 | 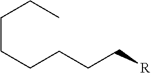 | 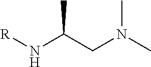 |

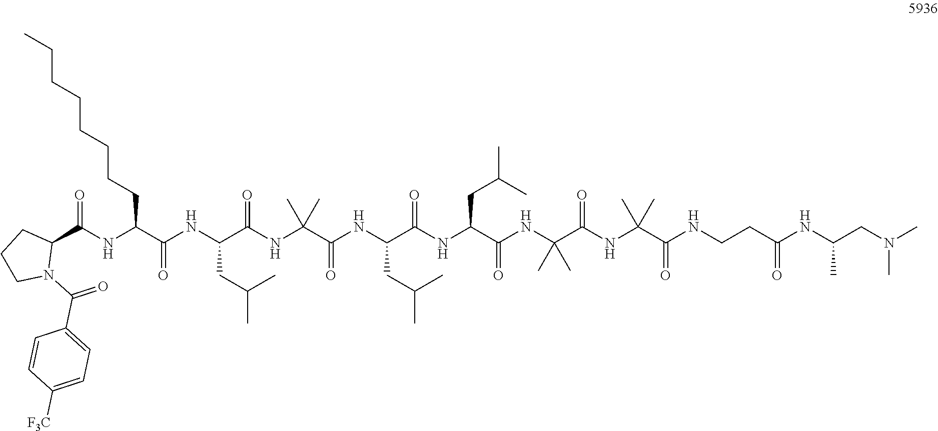 [SMILES: CCCCCCCC[C@H](NC(=O)[C@@H]1CCCN1C(=O)c1ccc(C(F)(F)F)cc1)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)N[C@@H](C)CN(C)C]

(2S)-N-[(1S)-1-[(1-{[(1S)-1-{[(1S)-1-{[1-({1-[(2-{[(2S)-1-(dimethylamino)propan-2-yl]carbamoyl}ethyl)carbamoyl]-1-methylethyl}carbamoyl)-1-methylethyl]carbamoyl}-3-methylbutyl]carbamoyl}-3-methylbutyl]carbamoyl}-1-methylethyl)carbamoyl]-3-methylbutyl]-2-{[(2S)-1-[4-(trifluoromethyl)benzoyl]pyrrolidin-2-yl]formamido}decanamide.

| Cpd | R1 | R2 | R3 |
|---|---|---|---|
| 5936 | 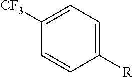 | 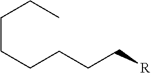 | 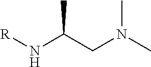 |

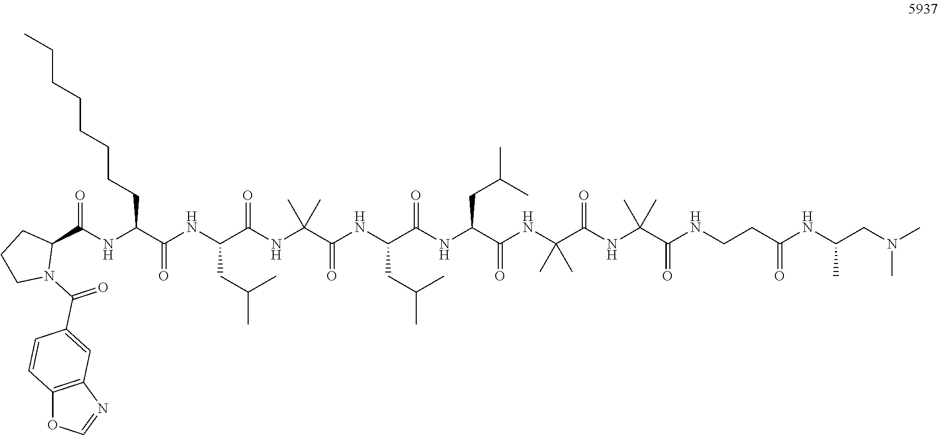 [SMILES: CCCCCCCC[C@H](NC(=O)[C@@H]1CCCN1C(=O)c1ccc2ocnc2c1)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)N[C@@H](C)CN(C)C]

(2S)-2-{[(2S)-1-(1,3-benzoxazole-5-carbonyl)pyrrolidin-2-yl]formamido}-N-[(1S)-1-[(1-{[(1S)-1-{[(1S)-1-{[1-({1-[(2-{[(2S)-1-(dimethylamino)propan-2-yl]carbamoyl}ethyl)carbamoyl]-1-methylethyl}carbamoyl)-1-methylethyl]carbamoyl}-3-methylbutyl]carbamoyl}-3-methylbutyl]carbamoyl}-1-methylethyl)carbamoyl]-3-methylbutyl]decanamide.

| Cpd | R1 | R2 | R3 |
|---|---|---|---|
| 5937 | 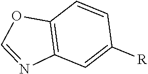 | 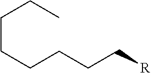 | 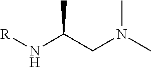 |

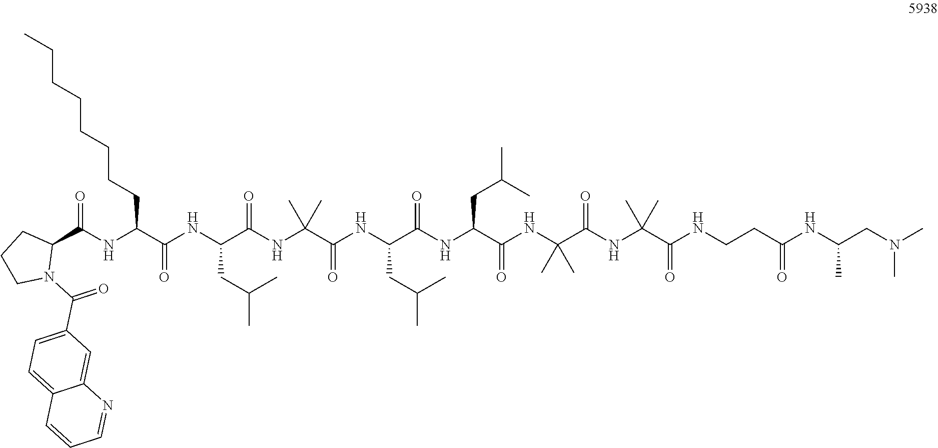 [SMILES: CCCCCCCC[C@H](NC(=O)[C@@H]1CCCN1C(=O)c1ccc2cccnc2c1)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)N[C@@H](C)CN(C)C]

(2S)-N-[(1S)-1-[(1-{[(1S)-1-{[(1S)-1-{[1-({1-[(2-{[(2S)-1-(dimethylamino)propan-2-yl]carbamoyl}ethyl)carbamoyl]-1-methylethyl}carbamoyl)-1-methylethyl]carbamoyl}-3-methylbutyl]carbamoyl}-3-methylbutyl]carbamoyl}-1-methylethyl)carbamoyl]-3-methylbutyl]-2-{[(2S)-1-(quinoline-7-carbonyl)pyrrolidin-2-yl]formamido}decanamide.

| Cpd | R1 | R2 | R3 |
|---|---|---|---|
| 5938 | 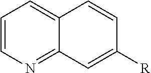 | 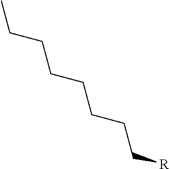 | 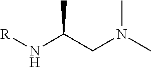 |

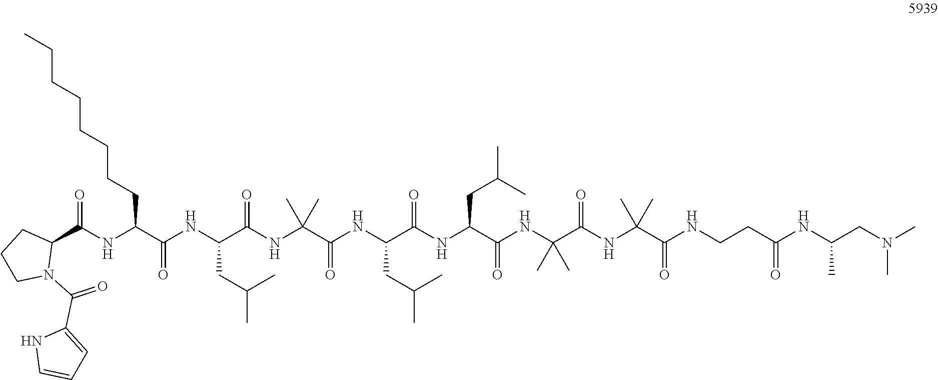 [SMILES: CCCCCCCC[C@H](NC(=O)[C@@H]1CCCN1C(=O)c1ccc[nH]1)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)N[C@@H](C)CN(C)C]

(2S)-N-[(1S)-1-[(1-{[(1S)-1-{[(1S)-1-{[1-({1-[(2-{[(2S)-1-(dimethylamino)propan-2-yl]carbamoyl}ethyl)carbamoyl]-1-methylethyl}carbamoyl)-1-methylethyl]carbamoyl}-3-methylbutyl]carbamoyl}-3-methylbutyl]carbamoyl}-1-methylethyl)carbamoyl]-3-methylbutyl]-2-{[(2S)-1-(1H-pyrrole-2-carbonyl)pyrrolidin-2-yl]formamido}decanamide.

| Cpd | R1 | R2 | R3 |
|---|---|---|---|
| 5939 | 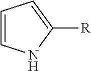 | 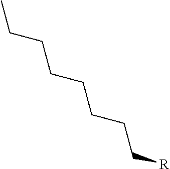 | 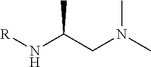 |

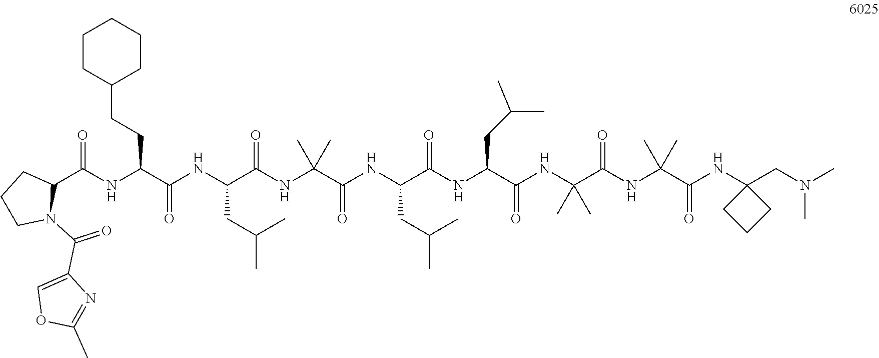

(2S)-2-[(2S)-4-cyclohexyl-2-{[(2S)-1-(2-methyl-1,3-oxazole-4-carbonyl)pyrrolidin-2-yl]formamido}butanamido]-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide

| Cpd | R1 | R2 | R3 |
|---|---|---|---|
| 6025 | 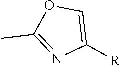 | 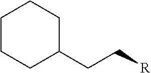 | 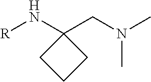 |

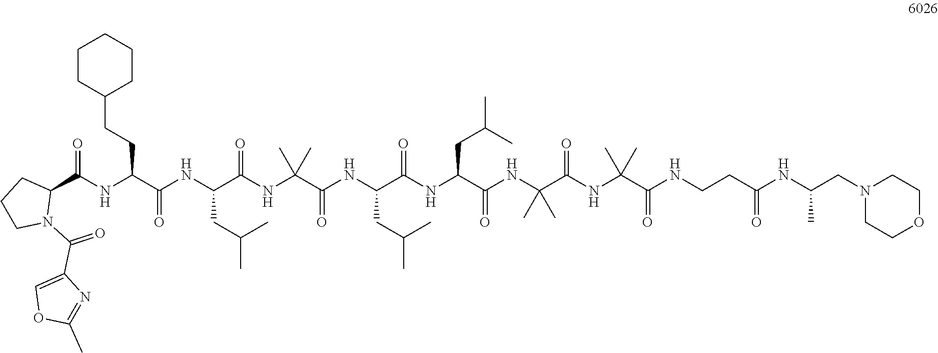 [SMILES: Cc1nc(C(=O)N2CCC[C@H]2C(=O)N[C@@H](CCC2CCCCC2)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)N[C@@H](C)CN2CCOCC2)co1]

(2S)-2-[(2S)-2-{2-[(2S)-2-[(2S)-4-cyclohexyl-2-{[(2S)-1-(2-methyl-1,3-oxazole-4-carbonyl)pyrrolidin-2-yl]formamido}butanamido]-4-methylpentanamido]-2-methylpropanamido}-4-methylpentanamido]-4-methyl-N-[1-methyl-1 ({1-methyl-1-[(2-{[(2S)-1-(morpholin-4-yl)propan-2-yl]carbamoyl}ethyl)carbamoyl]ethyl}carbamoyl)ethyl]pentanamide.

| Cpd | R1 | R2 | R3 |
|---|---|---|---|
| 6026 | 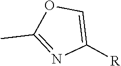 | 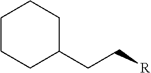 | 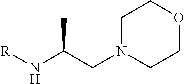 |

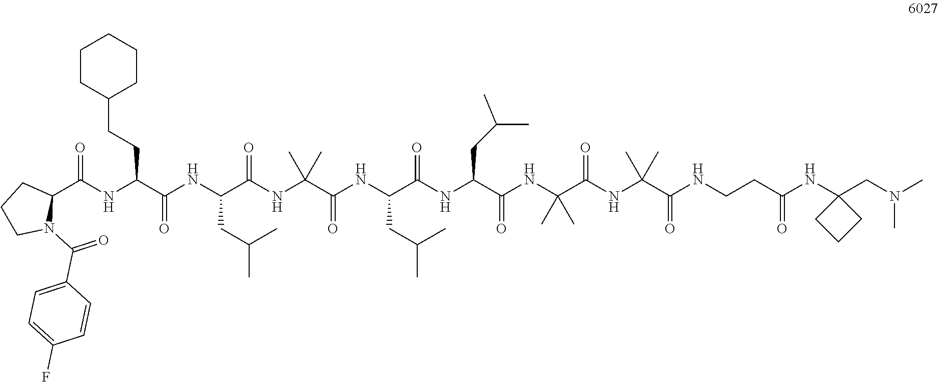 [SMILES: CC(C)C[C@H](NC(=O)[C@H](CCC1CCCCC1)NC(=O)[C@@H]1CCCN1C(=O)c1ccc(F)cc1)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NC1(CN(C)C)CCC1]

(2S)-2-[(2S)-4-cyclohexyl-2-{[(2S)-1-(4-fluorobenzoyl)pyrrolidin-2-yl]formamido}butanamido]-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide

| Cpd | R1 | R2 | R3 |
|---|---|---|---|
| 6027 | 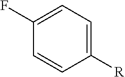 | 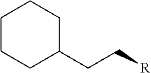 | 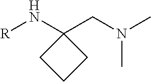 |

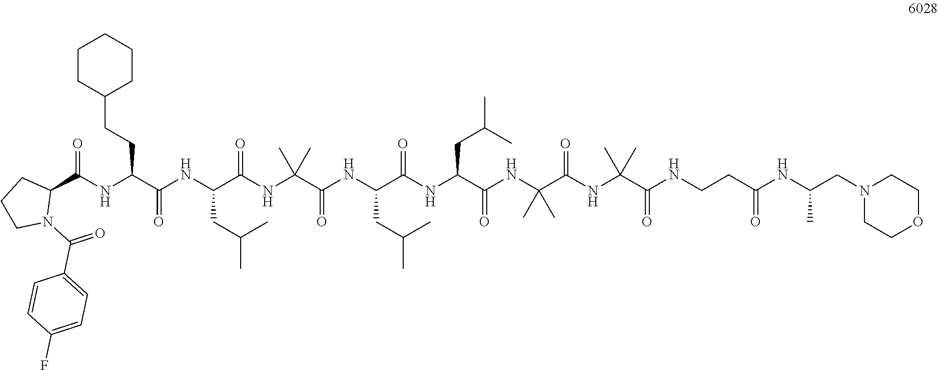 [SMILES: CC(C)C[C@H](NC(=O)[C@H](CCC1CCCCC1)NC(=O)[C@@H]1CCCN1C(=O)c1ccc(F)cc1)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)N[C@@H](C)CN1CCOCC1]

(2S)-2-[(2S)-2-{2-[(2S)-2-[(2S)-4-cyclohexyl-2-{[(2S)-1-(4-fluorobenzoyl)pyrrolidin-2-yl]formamido}butanamido]-4-methylpentanamido]-2-methylpropanamido}-4-methylpentanamido]-4-methyl-N-[1-methyl-1-({1-methyl-1-[(2-[(2S)-1-(morpholin-4-yl)propan-2-yl]carbamoyl}ethyl)carbamoyl]ethyl carbamoyl)ethyl]pentanamide.

| Cpd | R1 | R2 | R3 |
|---|---|---|---|
| 6028 | 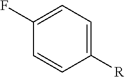 | 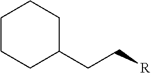 | 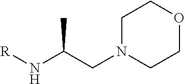 |

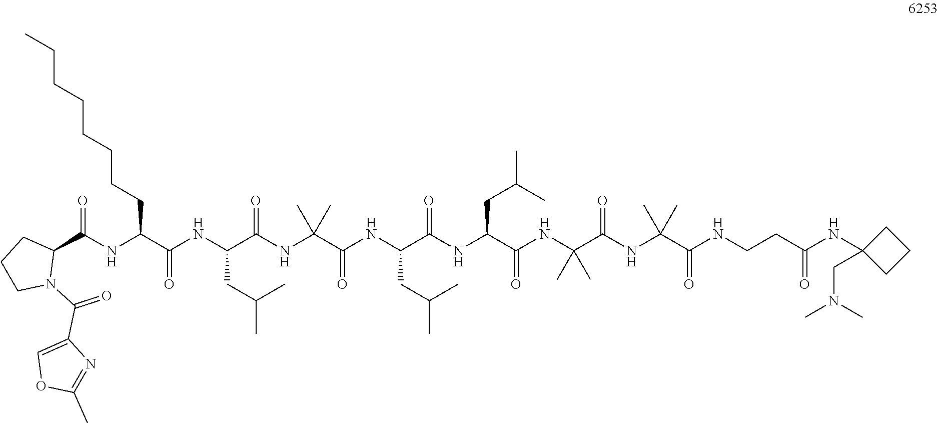 [SMILES: CCCCCCCC[C@H](NC(=O)[C@@H]1CCCN1C(=O)c1coc(C)n1)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NC1(CN(C)C)CCC1]

(2S)—N-[(1S)-1-[(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)carbamoyl]-3-methylbutyl]-2-{[(2S)-1-(2-methyl-1,3-oxazole-4-carbonyl)pyrrolidin-2-yl]formamido}decanamide

| Cpd | R1 | R2 | R3 |
|---|---|---|---|
| 6253 | 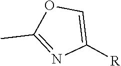 | 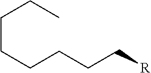 | 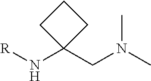 |

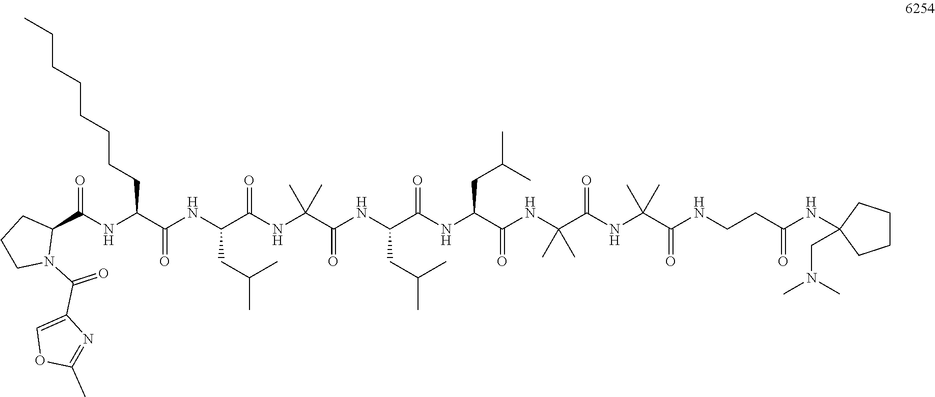 [SMILES: CCCCCCCC[C@H](NC(=O)[C@@H]1CCCN1C(=O)c1coc(C)n1)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NC1(CN(C)C)CCCC1]

(2S)—N-[(1S)-1-[(1-{[(1S)-1-{[(1S)-1-({1-[(1-{[2-({1-[(dimethylamino)methyl]cyclopentyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)carbamoyl]-3-methylbutyl]-2-{[(2S)-1-(2-methyl-1,3-oxazole-4-carbonyl)pyrrolidin-2-yl]formamido}decanamide.

| Cpd | R1 | R2 | R3 |
|---|---|---|---|
| 6254 | 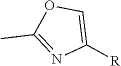 | 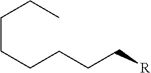 | 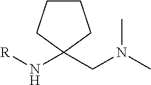 |

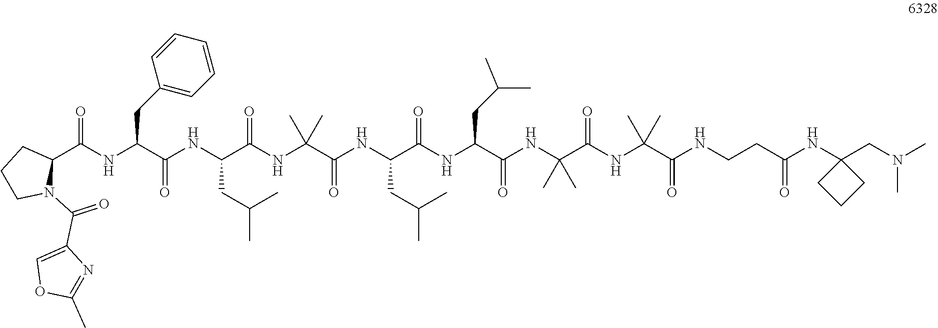 [SMILES: Cc1nc(C(=O)N2CCC[C@H]2C(=O)N[C@@H](Cc2ccccc2)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NC2(CN(C)C)CCC2)co1]

(2S)-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methyl-2-[(2S)-2-{[(2S)-1-(2-methyl-1,3-oxazole-4-carbonyl)pyrrolidin-2-yl]formamido}-3-phenylpropanamido]pentanamide

| Cpd | 1 | 2 | 3 | 4 | 5 |
|---|---|---|---|---|---|
| 6328 | Fmoc-Aib- | Fmoc-Aib- | Fmoc-Leu- | Fmoc-Leu- | Fmoc-Aib- |
| | OH | OH | OH | OH | OH |

| 6 | 7 | 8 | 9 | 10 |
|---|---|---|---|---|
| Fmoc-Leu- | Fmoc-Phe- | Fmoc-Pro- | 2- | 1-[(dimethylamino) |
| OH | OH | OH | methyloxazole- | methyl]cyclobutan- |
| | | | 4-carboxylic | 1-amine |
| | | | acid | |

| Cpd | Molecular mass | Experimentally measured mass |
|---|---|---|
| 6328 [M + H]+ | 1147.72 | 1147.76 |

| Cpd | R1 | R2 | R3 |
|---|---|---|---|
| 6328 | 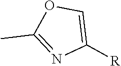 | 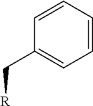 | 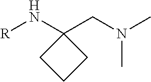 |

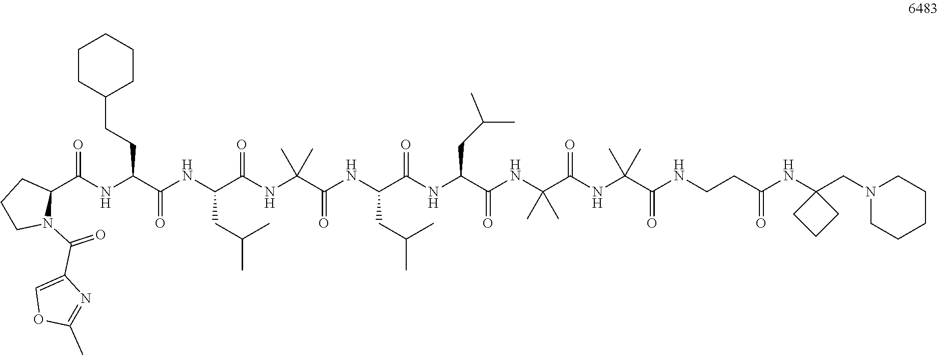 [SMILES: Cc1nc(C(=O)N2CCC[C@H]2C(=O)N[C@@H](CCC2CCCCC2)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NC2(CN3CCCCC3)CCC2)co1]

(2S)-2-[(2S)-4-cyclohexyl-2-{[(2S)-1-(2-methyl-1,3-oxazole-4-carbonyl)pyrrolidin-2-yl]formamido}butanamido]-4-methyl-N-(1-methyl-1-{[(1S)-3-methyl-1-{[(1S)-3-methyl-1-(}1-methyl-1-[(1-methyl-1-{[2-({1-[(piperidin-1-yl)methyl]cyclobutyl 1 carbamoyl)ethyl]carbamoyl 1 ethyl)carbamoyl]ethyl}carbamoyl)butyl]carb amyl}butyl]carbamoyl}ethyl)pentanamide

| Cpd | 1 | 2 | 3 | 4 | 5 |
|---|---|---|---|---|---|
| 6483 | Fmoc-Aib- | Fmoc-Aib- | Fmoc-Leu- | Fmoc-Leu- | Fmoc-Aib- |
| | OH | OH | OH | OH | OH |

| 6 | 7 | 8 | 9 | 10 |
|---|---|---|---|---|
| Fmoc-Leu- | Fmoc-L- | Fmoc-Pro- | 2- | 1-(piperidin-1- |
| OH | homocyclohexylalanine | OH | methyloxazole- | ylmethyl)cyclobutan- |
| | | | 4-carboxylic | 1-amine |
| | | | acid | dihydrochloride |

| Cpd | Molecular mass | Experimentally measured mass |
|---|---|---|
| 6384 [M + H] + | 1207.82 | 1208.03 |

| Cpd | R1 | R2 | R3 |
|---|---|---|---|
| 6483 | 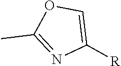 | 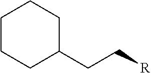 | 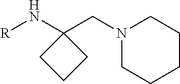 |

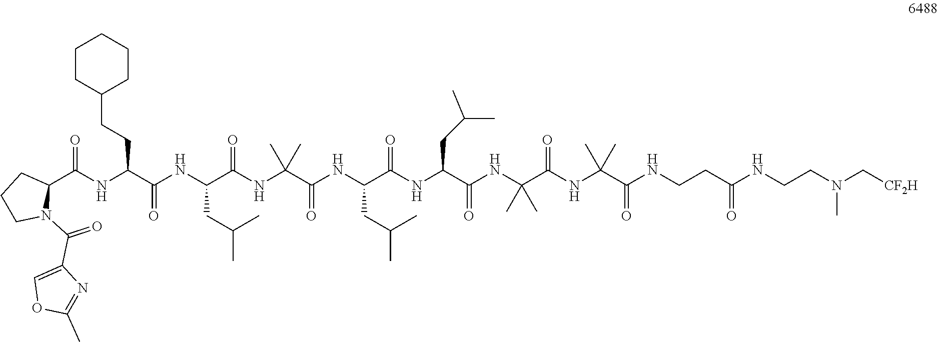 [SMILES: Cc1nc(C(=O)N2CCC[C@H]2C(=O)N[C@@H](CCC2CCCCC2)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NCCN(C)CC(F)F)co1]

(2S)-2-[(2S)-4-cyclohexyl-2-{[(2S)-1-(2-methyl-1,3-oxazole-4-carbonyl)pyrrolidin-2-yl]formamido}butanamido]-N-(1-{[(1S)-1-{[(1S)-1-({1-[(1-{[2-({2-[(2,2-difluoroethyl)(methyl)amino]ethyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide

| Cpd | 1 | 2 | 3 | 4 | 5 |
|---|---|---|---|---|---|
| 6488 | Fmoc-Aib- | Fmoc-Aib- | Fmoc-Leu- | Fmoc-Leu- | Fmoc-Aib- |
| | OH | OH | OH | OH | OH |

| 6 | 7 | 8 | 9 | 10 |
|---|---|---|---|---|
| Fmoc- | Fmoc-L- | Fmoc-Pro- | 2- | (2,2- |
| Leu-OH | homocyclohexylalanine | OH | methyloxazole- | difluoro- |
| | | | 4-carboxylic | ethyl)- |
| | | | acid | methyl- |
| | | | | amine |

| Cpd | Molecular mass | Experimentally measured mass |
|---|---|---|
| 6488 [M + H]+ | 1177.75 | 1177.86 |

| Cpd | R1 | R2 | R3 |
|---|---|---|---|
| 6488 | 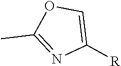 | 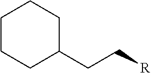 | 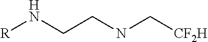 |

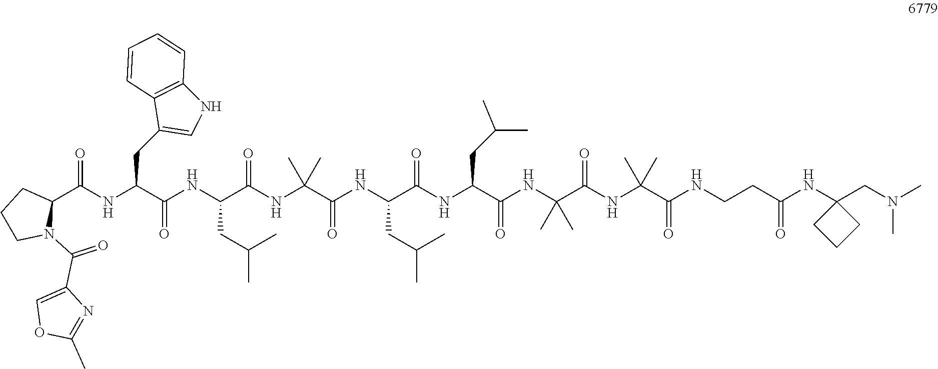 [SMILES: Cc1nc(C(=O)N2CCC[C@H]2C(=O)N[C@@H](Cc2c[nH]c3ccccc23)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NC2(CN(C)C)CCC2)co1]

(2S)-N-(1-{[(1 S)-1-{[(1S)-1-({1[(1{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-2-[(2S)-3-(1H-indol-3-yl)-2-{[(2S)-1-(2-methyl-1,3-oxazole-4-carbonyl)pyrrolidin-2-yl]formamido}propanamido]-4-methylpentanamide

| Cpd | 1 | 2 | 3 | 4 | 5 |
|---|---|---|---|---|---|
| 6779 | Fmoc-Aib- | Fmoc-Aib- | Fmoc-Leu- | Fmoc-Leu- | Fmoc-Aib- |
| | OH | OH | OH | OH | OH |

| 6 | 7 | 8 | 9 | 10 |
|---|---|---|---|---|
| Fmoc-Leu- | Fmoc-Trp- | Fmoc-Pro- | 2-methyloxazole-4- | 1-[(dimethylamino) |
| OH | OH | OH | carboxylic acid | methyl]cyclobutan- |
| | | | | 1-amine |

| Cpd | Molecular mass | Experimentally measured mass |
|---|---|---|
| 6779 [M + H]+ | 1186.74 | 1186.68 |

| Cpd | R1 | R2 | R3 |
|---|---|---|---|
| 6779 | 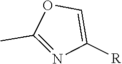 | 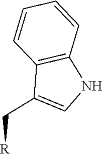 | 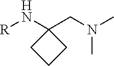 |

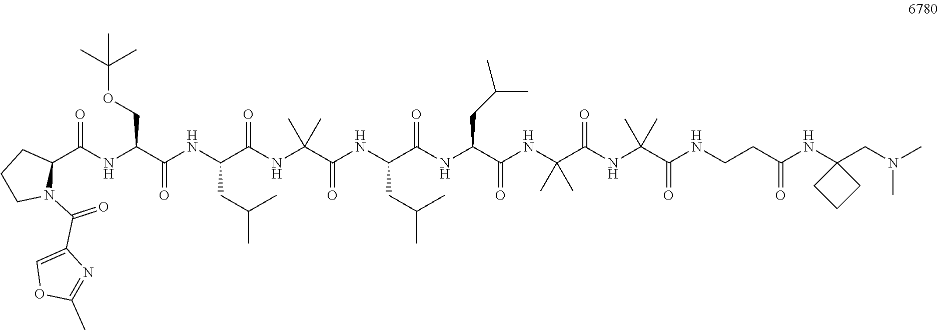 [SMILES: Cc1nc(C(=O)N2CCC[C@H]2C(=O)N[C@@H](COC(C)(C)C)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NC2(CN(C)C)CCC2)co1]

(2S)-2-[(2S)-2-{2-[(2S)-2-[(2S)-3-(tert-butoxy)-2-{[(2S)-1-(2-methyl-1,3-oxazole-4-carbonyl)pyrrolidin-2-yl]formamido}propanamido]-4-methylpentanamido]-2-methylpropanamido}-4-methylpentanamido]-N-{1-[(1-{[2-({1-[(dim ethyl amino)methyl]cyclobutyl}carbamoyl)ethyl]carbamoyl}-1-methylethyl)carbamoyl]-1-methylethyl}-4-methylpentanamide

| Cpd | 1 | 2 | 3 | 4 | 5 |
|---|---|---|---|---|---|
| 6780 | Fmoc-Aib- | Fmoc-Aib- | Fmoc-Leu- | Fmoc-Leu- | Fmoc-Aib- |
| | OH | OH | OH | OH | OH |

| 6 | 7 | 8 | 9 | 10 |
|---|---|---|---|---|
| Fmoc-Leu- | Fmoc- | Fmoc-Pro- | 2-methyloxazole- | 1-[(dimethylamino) |
| OH | Ser(tBu)-OH | OH | 4-carboxylic acid | methyl]cyclobutan- |
| | | | | 1-amine |

| Cpd | Molecular mass | Experimentally measured mass |
|---|---|---|
| 6780 [M + H]+ | 1143.75 | 1143.56 |

| Cpd | R1 | R2 | R3 |
|---|---|---|---|
| 6780 | 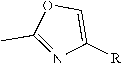 | 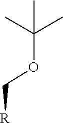 | 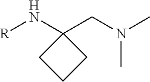 |

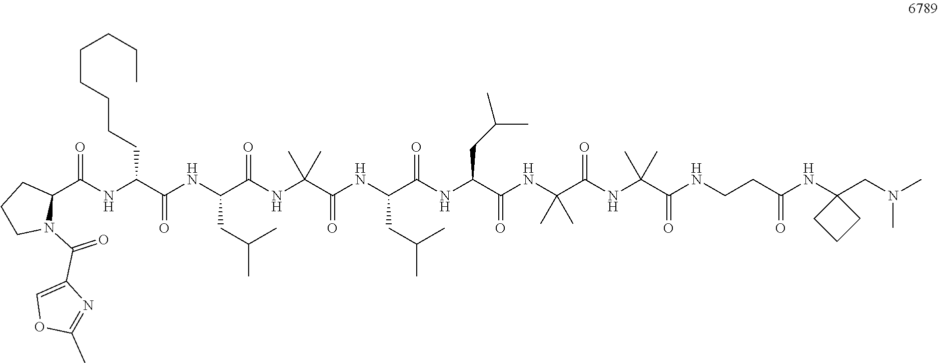 [SMILES: CCCCCCCC[C@@H](NC(=O)[C@@H]1CCCN1C(=O)c1coc(C)n1)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NC1(CN(C)C)CCC1]

(2R)-N-[(1S)-1-[(1-{[(1S)-1-{[(1S)-1-({1-[(1-{[2-({1-[(dim ethyl amino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)carbamoyl]-3-methylbutyl]-2-{[(2S)-1-(2-methyl-1,3-oxazole-4-carbonyl)pyrrolidin-2-yl]formamido}decanamide

| Cpd | 1 | 2 | 3 | 4 | 5 |
|---|---|---|---|---|---|
| 6789 | Fmoc-Aib- | Fmoc-Aib- | Fmoc-Leu- | Fmoc-Leu- | Fmoc-Aib- |
| | OH | OH | OH | OH | OH |

| 6 | 7 | 8 | 9 | 10 |
|---|---|---|---|---|
| Fmoc-Leu- | Fmoc—(R)-2- | Fmoc-Pro- | 2-methyloxazole- | 1-[(dimethylamino) |
| OH | amino-decanoic | OH | 4-carboxylic acid | methyl]cyclobutan- |
| | acid | | | 1-amine |

| Cpd | Molecular mass | Experimentally measured mass |
|---|---|---|
| 6789 [M + H]+ | 1169.80 | 1169.60 |

| Cpd | R1 | R2 | R3 |
|---|---|---|---|
| 6789 | 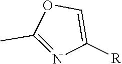 | 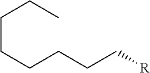 | 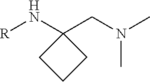 |

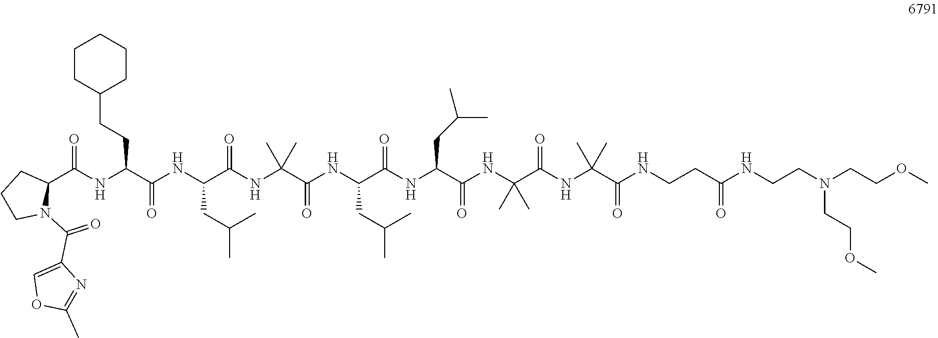 [SMILES: COCCN(CCNC(=O)CCNC(=O)C(C)(C)NC(=O)C(C)(C)NC(=O)[C@H](CC(C)C)NC(=O)[C@H](CC(C)C)NC(=O)C(C)(C)NC(=O)[C@H](CC(C)C)NC(=O)[C@H](CCC1CCCCC1)NC(=O)[C@@H]1CCCN1C(=O)c1coc(C)n1)CCOC]

(2S)-N-(1-{[(1S)-1-{[(1S)-1-({1-[(1-{[2-({2-[bis(2-methoxyethyl)amino]ethyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-2-[(2S)-4-cyclohexyl-2-{[(2S)-1-(2-methyl-1,3-oxazole-4-carbonyl)pyrrolidin-2-yl]formamido}butanamido]-4-methylpentanamide

| Cpd | 1 | 2 | 3 | 4 | 5 |
|---|---|---|---|---|---|
| 6791 | Fmoc-Aib- | Fmoc-Aib- | Fmoc-Leu- | Fmoc-Leu- | Fmoc-Aib- |
| | OH | OH | OH | OH | OH |

| 6 | 7 | 8 | 9 | 10 |
|---|---|---|---|---|
| Fmoc-Leu- | Fmoc-L- | Fmoc-Pro- | 2-methyloxazole- | (2-aminoethyl)bis(2- |
| OH | homocyclohexylalanine | OH | 4-carboxylic acid | methoxyethyl)amine |
| | | | | dihydrochloride |

| Cpd | Molecular mass | Experimentally measured mass |
|---|---|---|
| 6791 [M + H]+ | 1215.81 | 1215.73 |

| Cpd | R1 | R2 | R3 |
|---|---|---|---|
| 6791 | 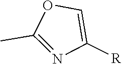 | 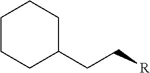 | 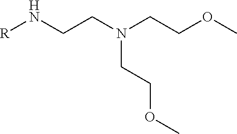 |

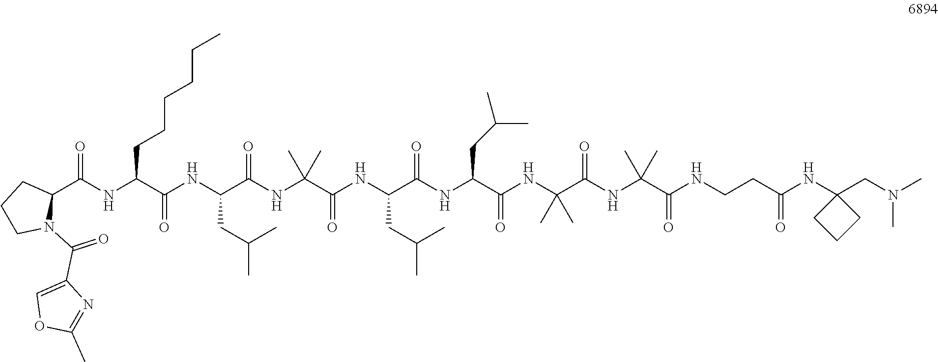 [SMILES: CCCCCC[C@H](NC(=O)[C@@H]1CCCN1C(=O)c1coc(C)n1)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NC1(CN(C)C)CCC1]

(2S)—N-[(1S)-1-[(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)carbamoyl]-3-methylbutyl]-2-{[(2S)-1-(2-methyl-1,3-oxazole-4-carbonyl)pyrrolidin-2-yl]formamido}octanamide

| Cpd | 1 | 2 | 3 | 4 | 5 |
|---|---|---|---|---|---|
| 6894 | Fmoc-Aib- | Fmoc-Aib- | Fmoc-Leu- | Fmoc-Leu- | Fmoc-Aib- |
| | OH | OH | OH | OH | OH |

| 6 | 7 | 8 | 9 | 10 |
|---|---|---|---|---|
| Fmoc-Leu- | Fmoc—(S)-2- | Fmoc-Pro- | 2-methyloxazole- | 1-[(dimethylamino) |
| OH | aminooctanoic | OH | 4-carboxylic acid | methyl]cyclobutan- |
| | acid | | | 1-amine |

| Cpd | Molecular mass | Experimentally measured mass |
|---|---|---|
| 6894 [M + H]+ | 1141.77 | 1141.88 |

| Cpd | R1 | R2 | R3 |
|---|---|---|---|
| 6894 | 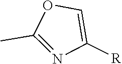 | 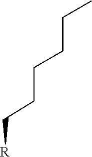 | 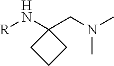 |

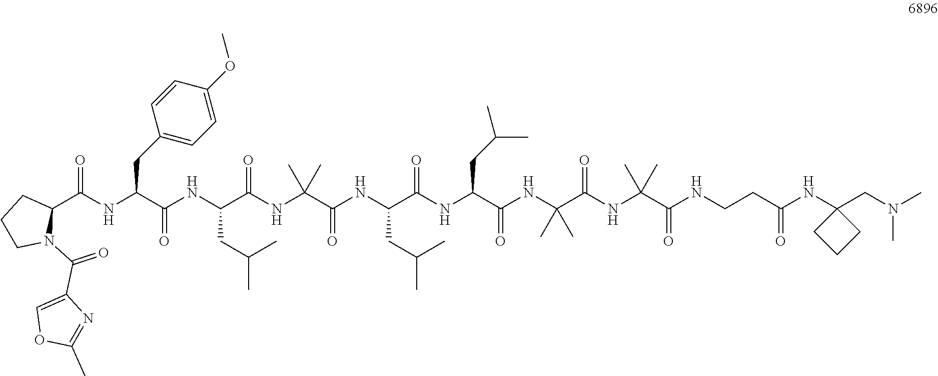 [SMILES: COc1ccc(C[C@H](NC(=O)[C@@H]2CCCN2C(=O)c2coc(C)n2)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NC2(CN(C)C)CCC2)cc1]

(2S)-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-2-[(2S)-3-(4-methoxyphenyl)-2-{[(2S)-1-(2-methyl-1,3-oxazole-4-carbonyl)pyrrolidin-2-yl]formamido}propanamido]-4-methylpentanamide

| Cpd | 1 | 2 | 3 | 4 | 5 |
|---|---|---|---|---|---|
| 6896 | Fmoc-Aib- | Fmoc-Aib- | Fmoc-Leu- | Fmoc-Leu- | Fmoc-Aib- |
| | OH | OH | OH | OH | OH |

| 6 | 7 | 8 | 9 | 10 |
|---|---|---|---|---|
| Fmoc-Leu- | Fmoc- | Fmoc-Pro- | 2-methyloxazole- | 1-[(dimethylamino) |
| OH | Tyr(Me)—OH | OH | 4-carboxylic acid | methyl]cyclobutan- |
| | | | | 1-amine |

| Cpd | Molecular mass | Experimentally measured mass |
|---|---|---|
| 6896 [M + H]+ | 1177.73 | 1177.72 |

| Cpd | R1 | R2 | R3 |
|---|---|---|---|
| 6896 | 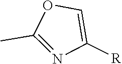 | 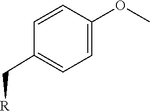 | 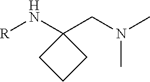 |

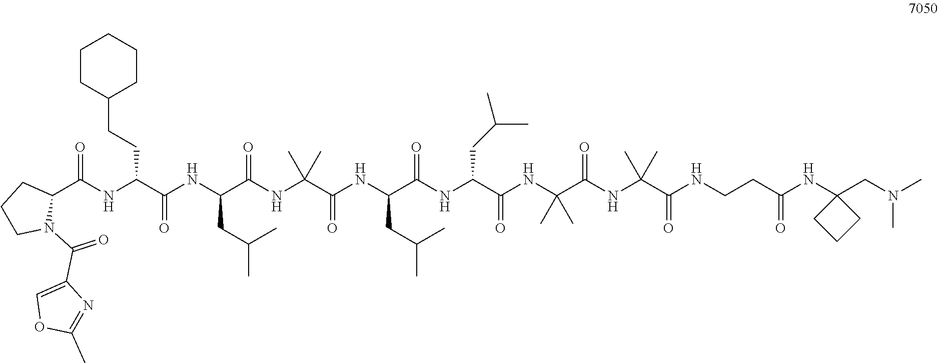 [SMILES: Cc1nc(C(=O)N2CCC[C@@H]2C(=O)N[C@H](CCC2CCCCC2)C(=O)N[C@H](CC(C)C)C(=O)NC(C)(C)C(=O)N[C@H](CC(C)C)C(=O)N[C@H](CC(C)C)C(=O)NC(C)(C)C(=O)NC(C)(C)C(=O)NCCC(=O)NC2(CN(C)C)CCC2)co1]

(2R)-2-[(2R)-4-cyclohexyl-2-{[(2R)-1-(2-methyl-1,3-ox azole-4-carbonyl)pyrrolidin-2-yl]formamido}butanamido]-N-(1-{[(1R)-1-{[(1R)-1-({1-[(1-{[2-({1-[(dim ethyl amino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide

| Cpd | 1 | 2 | 3 | 4 | 5 |
|---|---|---|---|---|---|
| 7050 | Fmoc-Aib- | Fmoc-Aib- | Fmoc-D- | Fmoc-D- | Fmoc-Aib- |
| | OH | OH | Leu-OH | Leu-OH | OH |

| 6 | 7 | 8 | 9 | 10 |
|---|---|---|---|---|
| Fmoc-D- | Fmoc-D- | Fmoc-D- | 2-methyloxazole- | 1-[(dimethylamino) |
| Leu-OH | homocyclohexylalanine | Pro-OH | 4-carboxylic acid | methyl]cyclobutan- |
| | | | | 1-amine |

| Cpd | Molecular mass | Experimentally measured mass |
|---|---|---|
| 7050 [M + H]⁺ | 1167.79 | 1167.64 |

| Cpd | R1 | R2 | R3 |
|---|---|---|---|
| 7050 | 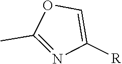 | 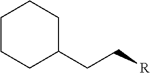 | 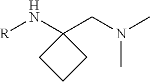 |

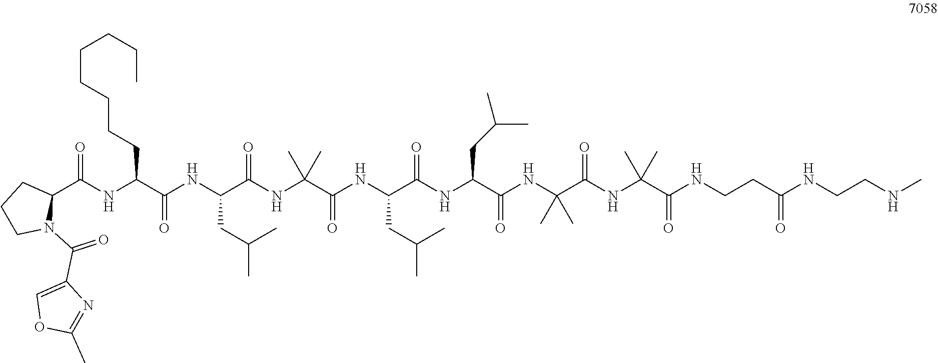 [SMILES: CCCCCCCC[C@H](NC(=O)[C@@H]1CCCN1C(=O)c1coc(C)n1)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NCCNC]

(2S)-2-{[(2S)-1-(2-methyl-1,3-oxazole-4-carbonyl)pyrrolidin-2-yl]formamido 1-N-[(1S)-3-methyl-1-[(1-methyl-1-{[(1S)-3-methyl-1-{[(1S)-3-methyl-1-1 [1-methyl-1-({1-methyl-1-[(2-{[2-(methyl amino)ethyl]carbamoyl}ethyl)carbamoyl]ethyl}carbamoyl) ethyl]carbamoyl}butyl]carbamoyl}butyl]carbamoyl}ethyl)carbamoyl]butyl]decanamide

| Cpd | 1 | 2 | 3 | 4 | 5 |
|---|---|---|---|---|---|
| 7058 | Fmoc-Aib- | Fmoc-Aib- | Fmoc-Leu- | Fmoc-Leu- | Fmoc-Aib- |
| | OH | OH | OH | OH | OH |

| 6 | 7 | 8 | 9 | 10 |
|---|---|---|---|---|
| Fmoc-Leu- | Fmoc—(S)-2- | Fmoc-Pro- | 2-methyloxazole- | tert-Butyl (2- |
| OH | amino-decanoic | OH | 4-carboxylic acid | aminoethyl) |
| | acid | | | (methyl) |
| | | | | carbamate |

| Cpd | Molecular mass | Experimentally measured mass |
|---|---|---|
| 7058 [M + H]+ | 1115.76 | 1115.70 |

| Cpd | R1 | R2 | R3 |
|---|---|---|---|
| 7058 | 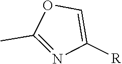 | 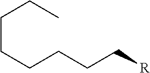 | 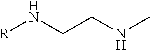 |

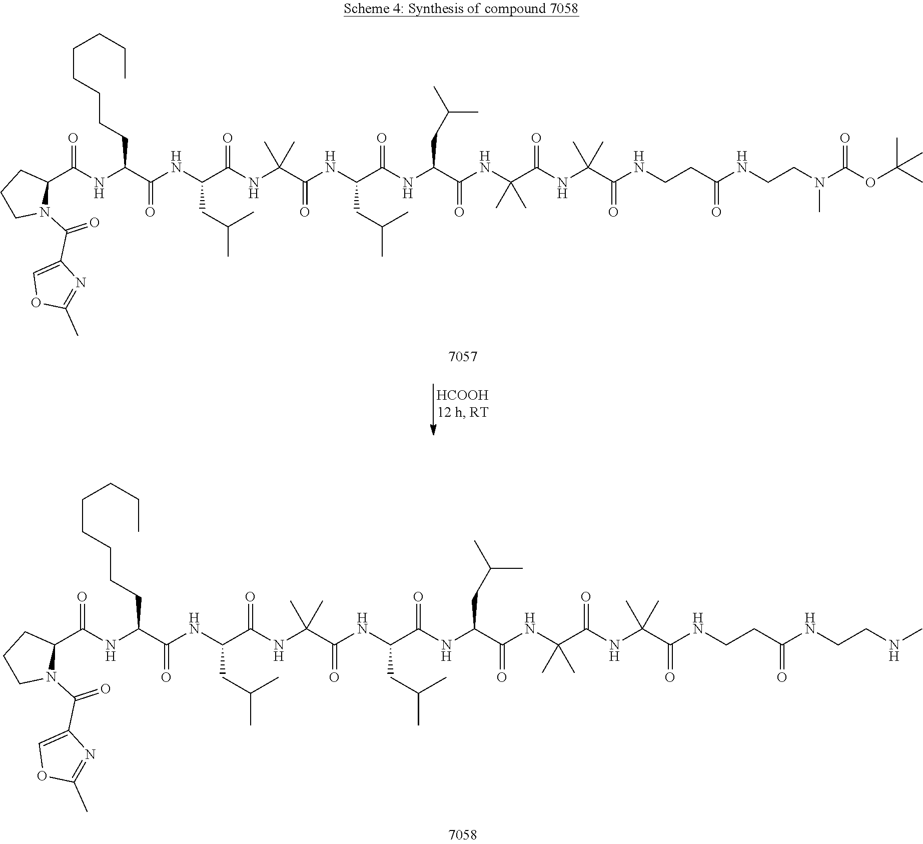 [SMILES: CCCCCCCC[C@H](NC(=O)[C@@H]1CCCN1C(=O)c1coc(C)n1)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NCCN(C)C(=O)OC(C)(C)C ; CCCCCCCC[C@H](NC(=O)[C@@H]1CCCN1C(=O)c1coc(C)n1)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NCCNC]

Compound 7058 was produced as depicted in Scheme 4. More analytically: Formic acid (3.7 mL) was added at compound 7057 (0.1 mmol) and the reaction mixture was left to stir for 12 h at RT. Upon completion, formic acid was removed under vacuo and 7058 was obtained without further purification.

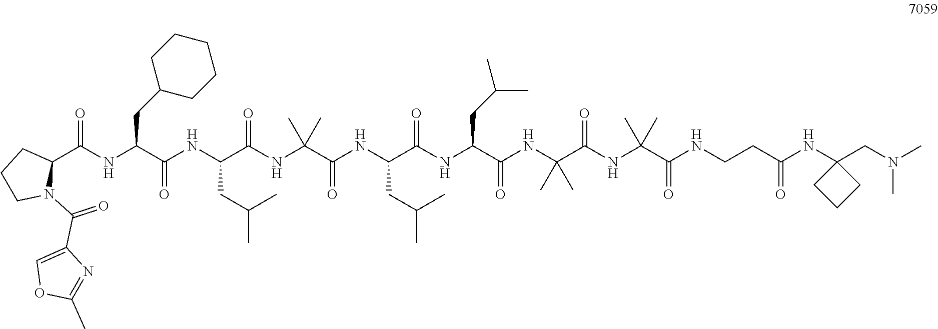 [SMILES: Cc1nc(C(=O)N2CCC[C@H]2C(=O)N[C@@H](CC2CCCCC2)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NC2(CN(C)C)CCC2)co1]

(2S)-2-[(2S)-3-cyclohexyl-2-{[(2S)-1-(2-methyl-1,3-oxazole-4-carbonyl)pyrrolidin-2-yl]formamido}propanamido]-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide

| Cpd | 1 | 2 | 3 | 4 | 5 |
|---|---|---|---|---|---|
| 7059 | Fmoc-Aib- | Fmoc-Aib- | Fmoc-Leu- | Fmoc-Leu- | Fmoc-Aib- |
| | OH | OH | OH | OH | OH |

| 6 | 7 | 8 | 9 | 10 |
|---|---|---|---|---|
| Fmoc-Leu- | Fmoc-Cha- | Fmoc-Pro- | 2-methyloxazole-4- | 1-[(dimethylamino) |
| OH | OH | OH | carboxylic acid | methyl]cyclobutan- |
| | | | | 1-amine |

| Cpd | Molecular mass | Experimentally measured mass |
|---|---|---|
| 7059 [M + H]+ | 1153.77 | 1153.50 |

| Cpd | R1 | R2 | R3 |
|---|---|---|---|
| 7059 | 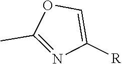 | 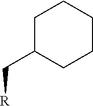 | 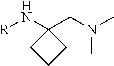 |

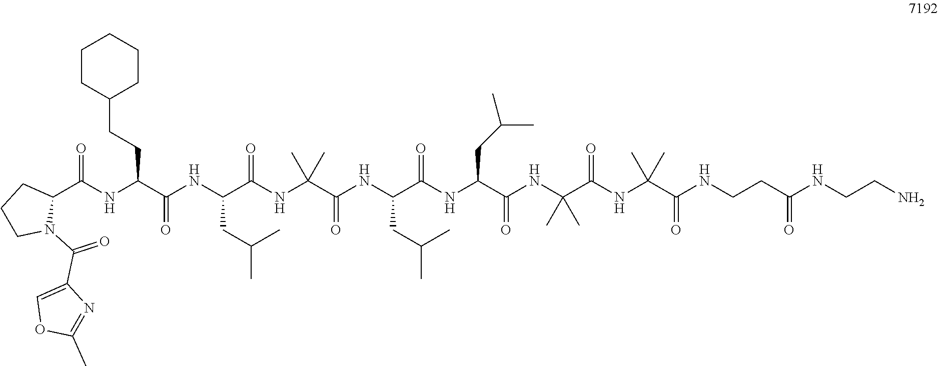 [SMILES: Cc1nc(C(=O)N2CCC[C@@H]2C(=O)N[C@@H](CCC2CCCCC2)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NCCN)co1]

(2S)-N-(1-{[(1S)-1-{[(1S)-1-[(1-{[1-({2-[(2-aminoethyl)carbamoyl]ethyl }carbamoyl)-1-methylethyl]carbamoyl}-1-methylethyl)carbamoyl]-3-methylbutyl]carbamoyl}-3-methylbutyl]carbamoyl}-1-methylethyl)-2-[(2S)-4-cyclohexyl-2-{[(2S)-1-(2-methyl-1,3-oxazole-4-carbonyl)pyrrolidin-2-yl]formamido}butanamido]-4-methylpentanamide

| Cpd | 1 | 2 | 3 | 4 | 5 |
|---|---|---|---|---|---|
| 7192 | Fmoc-Aib- | Fmoc-Aib- | Fmoc-Leu- | Fmoc-Leu- | Fmoc-Aib- |
| | OH | OH | OH | OH | OH |

| 6 | 7 | 8 | 9 | 10 |
|---|---|---|---|---|
| Fmoc-Leu- | Fmoc-L- | Fmoc-Pro- | 2-methyloxazole- | tert-Butyl (2- |
| OH | homocyclohexylalanine | OH | 4-carboxylic acid | aminoethyl) |
| | | | | carbamate |

| Cpd | Molecular mass | Experimentally measured mass |
|---|---|---|
| 7192 [M + H]+ | 1099.72 | 1099.46 |

| Cpd | R1 | R2 | R3 |
|---|---|---|---|
| 7192 | 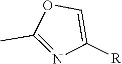 | 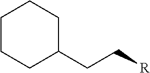 | 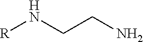 |

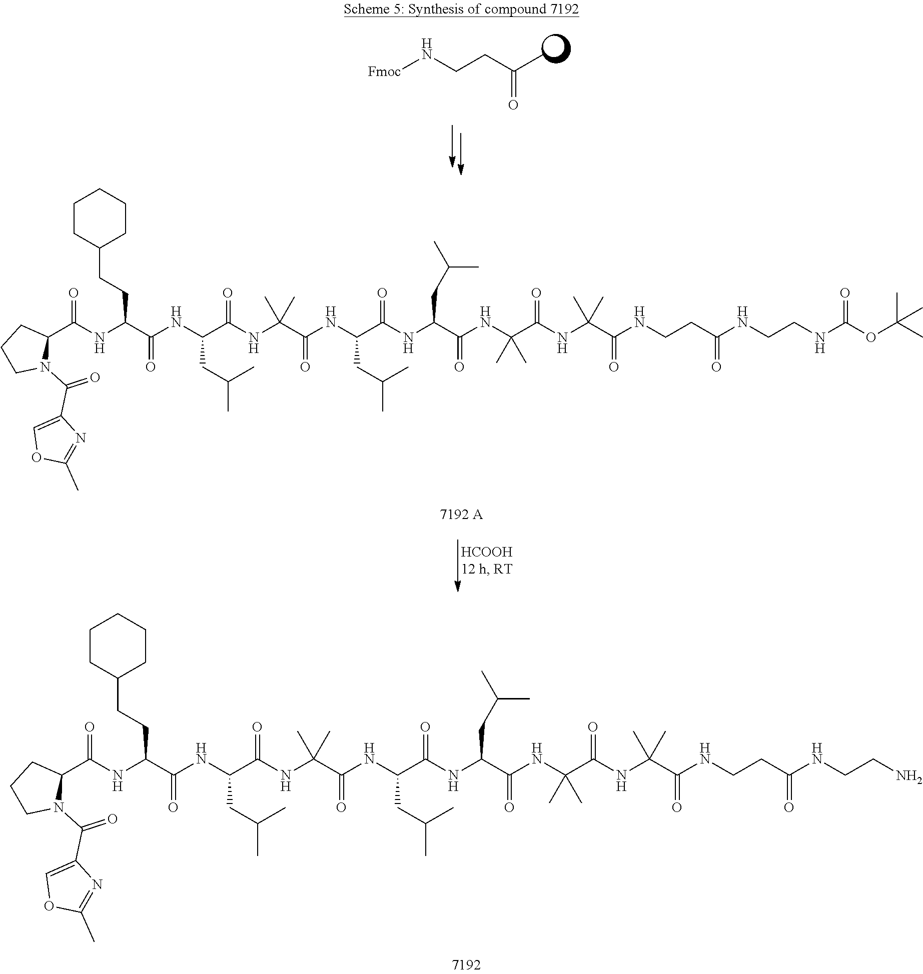

Compound 7192 was produced as depicted in Scheme 5. More analytically: 7192 A was synthesized according the general synthesis depicted at Scheme 1. The amino acids, acid and amine used for its synthesis are described at the table above. Afterwards, formic acid (6.3 mL) was added at compound 7192 A (0.1 mmol) and the reaction mixture was left to stir for 12 h, at RT. Upon completion, formic acid was removed under vacuo and 7192 was obtained without further purification.

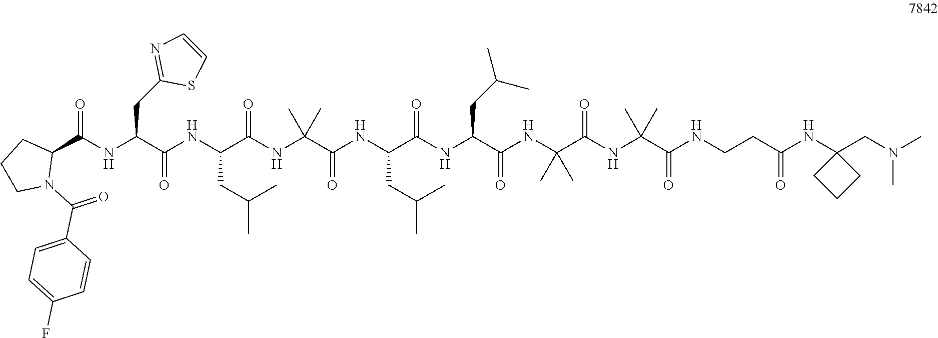 [SMILES: CC(C)C[C@H](NC(=O)[C@H](Cc1nccs1)NC(=O)[C@@H]1CCCN1C(=O)c1ccc(F)cc1)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NC1(CN(C)C)CCC1]

(2S)-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-2-[(2S)-2-{[(2S)-1-(4-fluorobenzoyl)pyrrolidin-2-yl]formamido}-3-(1,3-thiazol-2-yl)propanamido]-4-methylpentanamide

| Cpd | 1 | 2 | 3 | 4 | 5 |
|---|---|---|---|---|---|
| 7842 | Fmoc-Aib- | Fmoc-Aib- | Fmoc-Leu- | Fmoc-Leu- | Fmoc-Aib- |
| | OH | OH | OH | OH | OH |

| 6 | 7 | 8 | 9 | 10 |
|---|---|---|---|---|
| Fmoc-Leu- | Fmoc-4-Thi- | Fmoc-Pro- | 4- | 1-[(dimethylamino) |
| OH | OH | OH | Fluorobenzoic | methyl]cyclobutan- |
| | | | acid | 1-amine |

| Cpd | Molecular mass | Experimentally measured mass |
|---|---|---|
| 7842 [M + H]+ | 1167.68 | 1168.13 |

| Cpd | R1 | R2 | R3 |
|---|---|---|---|
| 7842 | 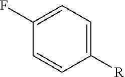 | 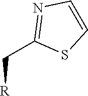 | 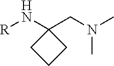 |

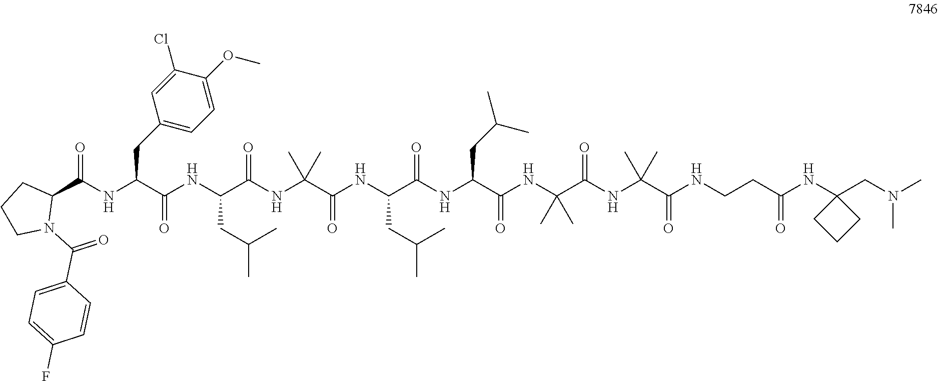 [SMILES: COc1ccc(C[C@H](NC(=O)[C@@H]2CCCN2C(=O)c2ccc(F)cc2)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NC2(CN(C)C)CCC2)cc1Cl]

(2S)-2-[(2S)-3-(3-chloro-4-methoxyphenyl)-2-{[(2S)-1-(4-fluorobenzoyl)pyrrolidin-2-yl]formamido}propanamido]-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-4-methylpentanamide

| Cpd | 1 | 2 | 3 | 4 | 5 |
|---|---|---|---|---|---|
| 7846 | Fmoc-Aib- | Fmoc-Aib- | Fmoc-Leu- | Fmoc-Leu- | Fmoc-Aib- |
| | OH | OH | OH | OH | OH |

| 6 | 7 | 8 | 9 | 10 |
|---|---|---|---|---|
| Fmoc-Leu- | (2S)-3-(3-chloro-4- | Fmoc-Pro- | 4- | 1-[(dimethylamino) |
| OH | methoxyphenyl)-2- | OH | Fluorobenzoic | methyl]cyclobutan- |
| | ({[(9H-fluoren-9-yl) | | acid | 1-amine |
| | methoxy]carbonyl} | | | |
| | amino)propanoic acid | | | |

| Cpd | Molecular mass | Experimentally measured mass |
|---|---|---|
| 7846 [M + H]+ | 1224.69 | 1225.39 |

| Cpd | R1 | R2 | R3 |
|---|---|---|---|
| 7846 | 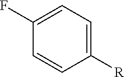 | 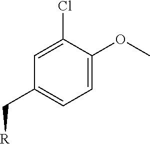 | 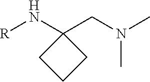 |

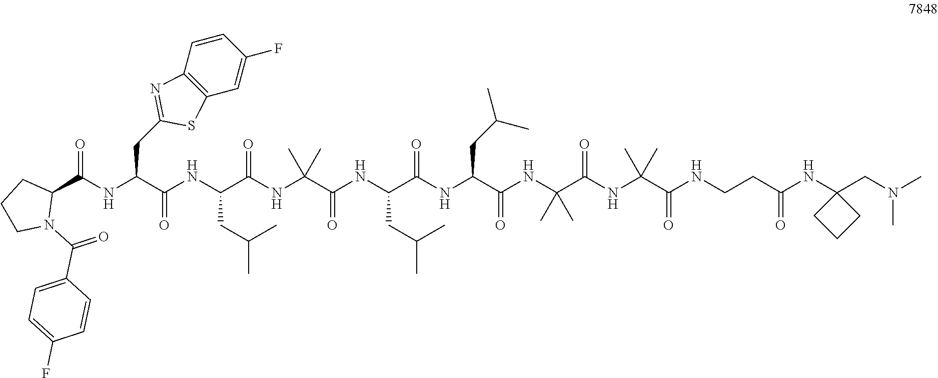 [SMILES: CC(C)C[C@H](NC(=O)[C@H](Cc1nc2ccc(F)cc2s1)NC(=O)[C@@H]1CCCN1C(=O)c1ccc(F)cc1)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NC1(CN(C)C)CCC1]

(2S)-N-(1-{[(1S)-1-{[(1S)-1-({1-[(1-{[2-({1-[(dimethylamino)methyl]cyclobutyl}carbamoyl)ethyl]carbamoyl}-1-methylethyl)carbamoyl]-1-methylethyl}carbamoyl)-3-methylbutyl]carbamoyl}-3-methylbutyl]carbamoyl}-1-methylethyl)-2-[(2S)-3-(6-fluoro-1,3-benzothiazol-2-yl)-2-{[(2S)-1-(4-fluorobenzoyl)pyrrolidin-2-yl]formamido}propanamido]-4-methylpentanamide

| Cpd | 1 | 2 | 3 | 4 | 5 |
|---|---|---|---|---|---|
| 7848 | Fmoc-Aib- | Fmoc-Aib- | Fmoc-Leu- | Fmoc-Leu- | Fmoc-Aib- |
| | OH | OH | OH | OH | OH |

| 6 | 7 | 8 | 9 | 10 |
|---|---|---|---|---|
| Fmoc-Leu- | (2S)-2-({[(9H-fluoren-9- | Fmoc-Pro- | 4- | 1-[(dimethylamino) |
| OH | yl)methoxy]carbonyl}amino)- | OH | Fluorobenzoic | methyl]cyclobutan- |
| | 3-(6-fluoro-1,3- | | acid | 1-amine |
| | benzothiazol-2-yl)propanoic | | | |
| | acid | | | |

| Cpd | Molecular mass | Experimentally measured mass |
|---|---|---|
| 7848 [M + H]+ | 1235.68 | 1236.38 |

| Cpd | R₁ | R₂ | R₃ |
|---|---|---|---|
| 7848 | 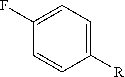 | 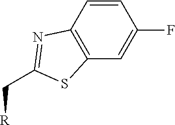 | 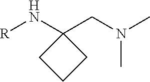 |

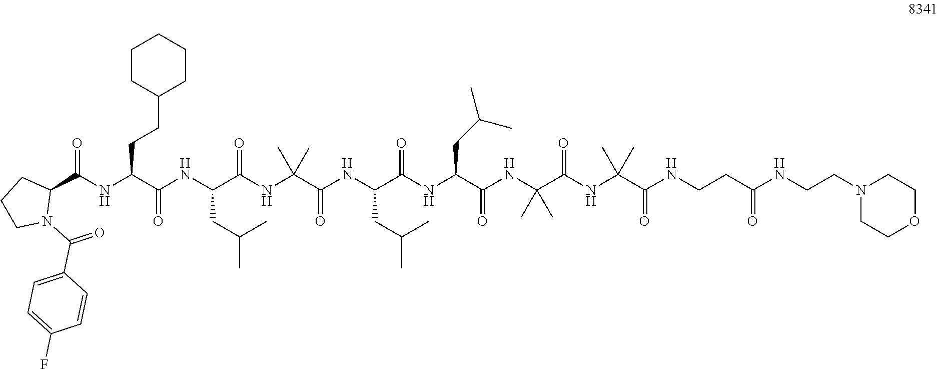 [SMILES: CC(C)C[C@H](NC(=O)[C@H](CCC1CCCCC1)NC(=O)[C@@H]1CCCN1C(=O)c1ccc(F)cc1)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NCCN1CCOCC1]

(2S)-2-[(2S)-4-cyclohexyl-2-{[(2S)-1-(4-fluorobenzoyl)pyrrolidin-2-yl]formamido}butanamido]-4-methyl-N-(1-methyl-1-{[(1S)-3-methyl-1-{[(1S)-3-methyl-1-{[1-methyl-1-({1-methyl-1-[(2-{[2-(morpholin-4-yl)ethyl]carbamoyl}ethyl)carbamoyl]ethyl}carbamoyl)ethyl]carbamoyl}butyl]carbamoyl}butyl]carbamoyl}ethyl)pentanamide

| Cpd | 1 | 2 | 3 | 4 | 5 |
|---|---|---|---|---|---|
| 8341 | Fmoc-Aib- | Fmoc-Aib- | Fmoc-Leu- | Fmoc-Leu- | Fmoc-Aib- |
| | OH | OH | OH | OH | OH |

| 6 | 7 | 8 | 9 | 10 |
|---|---|---|---|---|
| Fmoc-Leu- | Fmoc-L- | Fmoc-Pro- | 4- | 2-(morpholin- |
| OH | homocyclohexylalanine | OH | Fluorobenzoic | 4-yl)ethan-1- |
| | | | acid | amine |

| Cpd | Molecular mass | Experimentally measured mass |
|---|---|---|
| 8341 [M + H]+ | 1182.77 | 1183.04 |

| Cpd | R1 | R2 | R3 |
|---|---|---|---|
| 8341 | 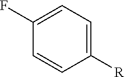 | 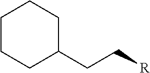 | 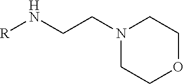 |

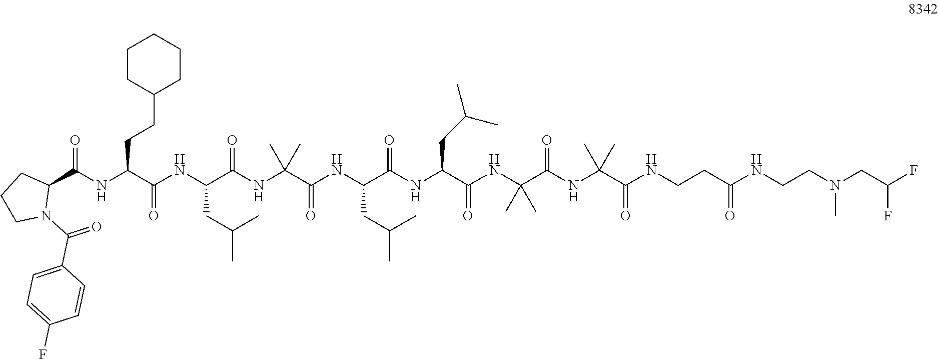 [SMILES: CC(C)C[C@H](NC(=O)[C@H](CCC1CCCCC1)NC(=O)[C@@H]1CCCN1C(=O)c1ccc(F)cc1)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NCCN(C)CC(F)F]

(2S)-2-[(2S)-4-cyclohexyl-2-{[(2S)-1-(4-fluorobenzoyl)pyrrolidin-2-yl]formamido}butanamido]-N-(1-{[(1S)-1-{[(1S)-1-({1-[(1-{[2-({2-[(2,2-difluoroethyl)(methyl)amino]ethyl}carbamoyl)ethyl]carbamoyl}-1-methylethyl)carbamoyl]-1-methylethyl}carbamoyl)-3-methylbutyl]carbamoyl}-methylbutyl]carbamoyl}-1-methylethyl)-4-methylpentanamide

| Cpd | 1 | 2 | 3 | 4 | 5 |
|---|---|---|---|---|---|
| 8342 | Fmoc-Aib- | Fmoc-Aib- | Fmoc-Leu- | Fmoc-Leu- | Fmoc-Aib- |
| | OH | OH | OH | OH | OH |

| 6 | 7 | 8 | 9 | 10 |
|---|---|---|---|---|
| Fmoc-Leu- | Fmoc-L- | Fmoc-Pro- | 4-Fluorobenzoic | (2,2-difluoro- |
| OH | homocyclohexyl | OH | acid | ethyl)-methyl- |
| | alanine | | | amine |

| Cpd | Molecular mass | Experimentally measured mass |
|---|---|---|
| 8342 [M + H]+ | 1190.75 | 1191.09 |

| Cpd | R1 | R2 | R3 |
|---|---|---|---|
| 8342 | 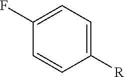 | 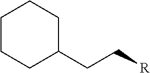 | 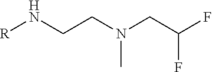 |

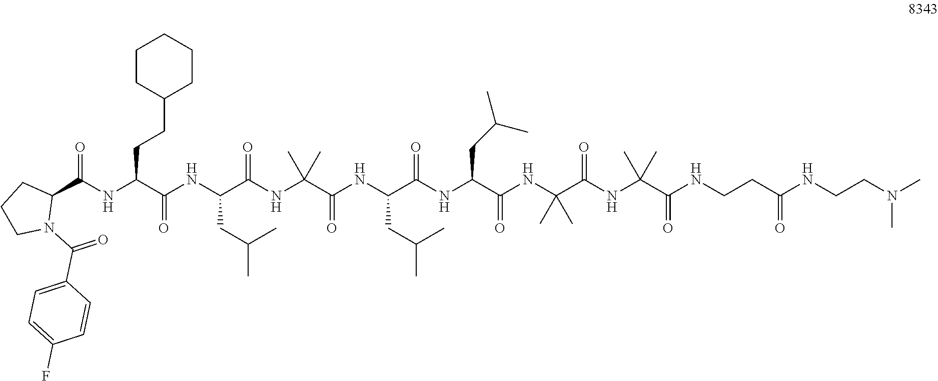 [SMILES: CC(C)C[C@H](NC(=O)[C@H](CCC1CCCCC1)NC(=O)[C@@H]1CCCN1C(=O)c1ccc(F)cc1)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NCCN(C)C]

(2S)-2-[(2S)-4-cyclohexyl-2-{[(28)-1-(4-fluorobenzoyl)pyrrolidin-2-yl]formamido}butanamido]-N-(1-{[(1S)-1-{[(1S)-1-{[1-({1-[(2-{[2-(dimethylamino)ethyl]carbamoyl}ethyl)carbamoyl]-1-methylethyl}carbamoyl)-1-methylethyl]carbamoyl}-3-methylbutyl]carbamoyl}-3-methylbutyl]carbamoyl}-1-methylethyl)-4-methylpentanamide

| Cpd | 1 | 2 | 3 | 4 | 5 |
|---|---|---|---|---|---|
| 8343 | Fmoc-Aib- | Fmoc-Aib- | Fmoc-Leu- | Fmoc-Leu- | Fmoc-Aib- |
| | OH | OH | OH | OH | OH |

| 6 | 7 | 8 | 9 | 10 |
|---|---|---|---|---|
| Fmoc-Leu- | Fmoc-L- | Fmoc-Pro- | 4- | (2-aminoethyl) |
| OH | homocyclohexylalanine | OH | Fluorobenzoic | dimethylamine |
| | | | acid | |

| Cpd | Molecular mass | Experimentally measured mass |
|---|---|---|
| 8343 [M + H]+ | 1140.75 | 1141.04 |

| Cpd | R₁ | R₂ | R₃ |
|---|---|---|---|
| 8343 | 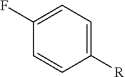 | 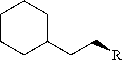 | 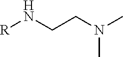 |

### Example 4

### Skin Permeation Studies

Skin permeating experiments were done with pig's skin using Franz Diffusion Cells (FDCs) using exemplarily the very preferred compound of the present invention 6027.

### Chemicals

Acetonitrile (ACN), formic acid (FA), Ethanol (EtOH) and dimethyl sulfoxide (DMSO) were all of HPLC grade and were together with Purified water was produced by reverse osmosis by Arium® pro ultrapure water system from Satorius Stedim Biotech GmbH (Gottingen, Germany).

### Skin Preparation

Skin from pig ears is widely used and accepted for skin permeation studies. Pig ears from freshly slaughtered pigs, not older than 5 months and of both sexes, were obtained from the slaughterhouse Bell Schweiz AG (Basel, Switzerland). The ears were washed with tap water and the hairs were removed with an electric clipper. The skin was separated from the underlying cartilage from the backside of the ear by using a scalpel. After washing the skin again, it was stored in aluminium foil at −20° C. for no longer than one month.

### Permeation Experiment

After thawing the frozen skin, it was cut into adequate circles and the skin thickness was measured with a calliper. The skin was mounted into the FDC, tightly sealed with Teflon band and fixed in special designed holder. Then the acceptor compartment was equipped with a magnetic stirrer, filled with placebo formulation and equilibrated for one hour. To check the barrier integrity and to be sure that there are no invisible damages from skin preparation TEWL (transepidermal water loss) was measured. The FDCs were then placed in the water bath +32° C. ±1 over a magnetic stirrer (500 rpm). Due to differences of each FDC, the skin application area for drug permeation was measured and calculated for each cell in advance. Consequently, the amount of 6027 for the donor compartment had to be adjusted for the infinite-dose experiment to 284.1 μL/cm².

At predefined time points 50 μL samples were taken from the acceptor compartment. The missing volume was replaced with the corresponding placebo formulation.

At the end of the permeation experiment and before reassembling the FDCs, the donor compartments were washed out with 10 mL ACN:H₂O (55:45).

### Skin Extraction

To extract 6027 from the skin, the frozen skin was cut into small pieces and grinded by a cryogenic grinder Freezer/Mill® from Spex SamplePrep (Metuchen, USA). The setting was 10 min precooling and a rate of 10 CPS for 4 cycles with 2 minutes. The resulting powder was resuspended in 2 mL ACN:H₂O (55:45). After being vortex, the mixtures were ultrasonicated with Branson Sonifier 250 (model: 101-cx03-197) from Branson Ultrasonics Corporation (Danbury, USA). The setting for ultrasonification was output control 2 and duty cycle 30% for 60 seconds. After shaking the samples with a horizontal shaker at 37° C. for 3 minutes, the skin powder was separated by centrifugation until the supernatant appeared clear. The extraction procedure was repeated two more times.

### High Performance Liquid Chromatography Mass Spectroscopy (HPLC-MS)

The concentrations of 6027 were determined by using HPLC-MS from Agilent Technologies. The system was equipped with Infinity LAB LC/MSD XT G6135B, a degasser G1379, an isocratic pump G1310A, an autosampler G1329A with thermostat G1330B, a column oven G1316 and a C18 reversed phase column Zobrax SB-C18 Narrow Bore 2.1×150 mm 5 Micron from Agilent.

The mobile phase was a mixture of ACN:H₂O:FA (55:45:0.1) [v/v]. The flow rate was set to 0.5 mL/min, the temperature in the autosampler was set to 25° C. and the column temperature was 50° C. The injection volume was 30 μL and the total runtime was 30 min. All samples were diluted with a mixture of ACN:H₂O (55:45) to be in the calibration range. Further conditions and details such as skin thickness are listed in Table 4.

TABLE 4
Quantification of 6027 in full thickness pigs skin using Franz cells
after 48 hours. Three replicates were done, and analysis was by
HPLC-MS after skin extraction. Data shown in % of total 6027.

| | | | | API |
|---|---|---|---|---|
| | volume | | volume | starting |
| | acceptor | medium | donor | amount |
| medium acceptor | [ml] | donor | [ml] | [mg] |
| PBS + 2% Volpo | 6 | 0.1% 6027 in | 0.5 | 0.5 |
| (Brij O20, | | DMSO | | |
| Polyalkylenglycolether) | | | | |

| | Total | | | Skin |
|---|---|---|---|---|
| skin thickness | recovery | Acceptor | Donor | extraction |
| [mm] | (%) | (%) | (%) | (%) |
| 1.4 | 95.2 | 0.2 | 79 | 16 |

A substantial skin penetration of 16% in FDC has been shown for 6027. This was in particular surprising since 6027, as well as the other compounds of formula (I), has not only a molecular weight above 1000 Da, but, moreover 6027 has a distribution coefficient octanol/water logD of 4.56 and a low water solubility of 0.022 mg/ml.

### Example 5

### In Vitro Antiprotozoal Assay

The testing of the inventive compounds against Leishmania donovani (strain MHOM/ET/67/L82) was performed in 96 well plates at concentrations in serial dilutions. Tests were done in triplicate. IC₅₀ values against the parasites as well as cytotoxic effects against rat myoblast (L6-cells) were determined by serial dilution and repeated twice.

The cytotoxicity assay was performed by a similar protocol as the Alamar Blue assay where L6-cells were seeded in 100 μL RPMI 1640 supplemented in 96-well micro titre plates (4000 cells/well). Podophyllotoxin (Sigma-Aldrich, Switzerland) was used as the reference drug (IC₅₀=0.05±0.01 μM). Its purity according to the supplier was more than 95%. After 68 h of incubation under humidified 5% CO₂ atmosphere, 10 μL of the Alamer Blue marker was added to all wells. The plates were incubated for additional 2 h. A Spectramax Gemini XS micro plate fluorescence reader (Molecular Devices Cooperation, Sunnyvale, CA) was used to measure the plates (Molecular Devices) using an excitation wavelength of 536 nm and an emission wavelength of 588 nm. The IC₅₀ were calculated by Softmax Pro software (Molecular Devices Cooperation, Sunnyvale, CA).

TABLE 5
Tested organisms, test conditions and reference compounds for the antiprotozoal
potency as well as cytotoxicity against rat myoblast cells (Cytotox L6)

| | | | | | IC₅₀ |
|---|---|---|---|---|---|
| | | | | Reference | Concurrent |
| Strain | Organism/Cells | Short form | Stage | compound | (nM) |
| MHOM-ET- | axenic Leishmania | L.don.ax. | amastigotes | Miltefosine | 162 |
| 67/L82 | donovani | | | | |
| MHOM-ET- | intracellular | L.don.InMac | amastigotes | Miltefosine | 162 |
| 67/L82 | Leishmania donovani | | | | |
| L6 | rat myoblast cells | Cytotox L6 | — | Podophyllotoxin | 19.3 |

TABLE 6
Antiprotozoal potency of compounds of formula (I) as
well as cytotoxicity against rat myoblast cells (Cytotox
L6). The values correspond to IC50 given in nM.

| Compound | L.don.ax. | L.don.InMac | Cytotox L6 |
|---|---|---|---|
| 5649 | 3.5 | 27.2 | 274.0 |
| 5768 | 33.5 | 257.3 | 627.4 |
| 5769 | 8.1 | 215.9 | 422.9 |
| 5770 | 1.0 | 338.4 | 185.1 |
| 5904 | 3.1 | 11.4 | 215.8 |
| 5905 | 7.9 | 13.1 | 851.8 |
| 5906 | 3.7 | 14.1 | 444.6 |
| 5910 | 2.1 | 6.7 | 230.2 |
| 5911 | 14.4 | 34.8 | 456.9 |
| 5912 | 5.1 | 12.8 | 253.7 |
| 5934 | 4.8 | <34.6 | 306.1 |
| 5936 | 3.3 | <33.2 | 410.7 |
| 5937 | 21.2 | 58.5 | 1161.5 |
| 5938 | 13.0 | | 713.7 |
| 5939 | 8.0 | | 611.1 |
| 6025 | 9.9 | 41.1 | 1910.0 |
| 6026 | 4.2 | 50.7 | 1584.3 |
| 6027 | 5.7 | 54.2 | 1342.6 |
| 6028 | 3.3 | 417.9 | 1592.1 |
| 6253 | 2.6 | | 944.8 |
| 6254 | 2.5 | | 527.6 |
| 6328 | 380.8 | | 8715.0 |
| 6483 | 4.1 | | 485.6 |
| 6488 | 428.9 | | 7176.4 |
| 6779 | 27.0 | 202.7 | 6679.4 |
| 6780 | 166.2 | 997.0 | 8745.4 |
| 6789 | 3.4 | 65.0 | 4279.5 |
| 6791 | 2.1 | 179.3 | 8226.6 |
| 6894 | 20.6 | 382.8 | 4743.8 |
| 6896 | 235.7 | 1638.1 | 8492.8 |
| 7050 | 5.1 | 43.7 | 4967.7 |
| 7058 | 23.8 | 2620.9 | 5044.2 |
| 7059 | 1.3 | | 6848.7 |
| 8341 | | | 7169 |
| 8342 | | | 6791 |
| 8343 | | | 6608 |

### Example 6

### In Vivo Antiprotozoal Assay

### Antileishmanial Effects In Vivo Model of Cutaneous Leishmaniasis (CL) Using the Rump Back Model in C67BLC/6 with Leishmania mexicana Infection

Mice. C67BLC/6 female mice were purchased from Envigo (Harlan laboratories) Indianapolis, IN, USA. 129SVE female mice were purchased from Taconic Farms, Cambridge City, IN, USA. All experimental mice were maintained at an OSU-ULAR facility in compliance with OSUIACUC.

Parasites and infection. L. mexicana (MNYC/BZ/62/m379) parasites were obtained and maintained as described previously. Promastigotes were maintained, passaged, and subsequently injected into experimental mice (5×10⁶ parasites) subcutaneously into their shaven rumps. C67BLC/6 mice were infected with L. mexicana parasites for 6 weeks after which they developed lesions on their back rumps. Mice with similar lesion sizes were selected and randomized into control and treatment groups. From the beginning of treatment animals were housed individually.

Treatment. Treatment group received 20 μl of ZHAW6027 0.1% weight by volume in DMSO, twice a day (bid) for 14 days. The test article was pipetted onto the lesion with a standard laboratory pipette. Control groups received 20 μl DMSO twice a day which was applied in the same way.

Topical drug treatments and lesion measurements. Lesion sizes were measured by using a small animal caliper twice weekly over the 14-day treatment duration and the area of the lesion was calculated by L×W, where L is the length and W is the width. FIG. 1 shows the cutaneous leishmaniasis (CL) lesion progression in L. mexicana infected C67BLC/6 mice. Measurements were done every 3 days by calliper over the 21-day treatment duration and shown in mm³ with standard deviation (SD). The volume of the induced CL lesions gradually reduced in the 6027 treated group over time, whereas lesions in the vehicle control group (DMSO) increased in volume.

Quantitation of parasitic load. After 2 weeks of treatment animals were sacrificed, and the parasite load in the lesions was quantified. Back rump lesions were harvested from all experimental mice, mashed in Schneider's medium supplemented with 10% FBS and 1% Penicillin/streptomycin. The suspensions were centrifuged at 3000 rpm and resuspended in 0.4 ml of Schneider's media. Viability of parasites was determined at 10 days of parasite culture. A limiting dilution assay was performed as previously described. The values reported represent the highest log dilution with viable parasites. The parasite loads in the 6027 treated group were reduced by about 3 orders of magnitude, compared to the control (FIG. 2).

### Example 7

### In Vitro Antibacterial Assay Method 1

The antibacterial activity of inventive compounds was determined in duplicate in a Minimum Inhibitory Concentration (MIC) assay.

The antibacterial activity of 6027 acetate was measured using the in vitro broth microdilution assay under assay conditions described by the Clinical and Laboratory Standards Institute. In this assay, the Minimum Inhibitory Concentration (MIC) is defined as the lowest concentration of an agent that completely inhibits visible growth in vitro of the microorganism. Pre-formulated DMSO stock solution (26.67 mg/mL) was provided by sponsor. The 26.67 mg/mL stock solution was then diluted in DMSO to prepare a 6.4 mg/mL working stock solution. The 6.4 mg/mL working stock solution was diluted by 2-fold serial titrations in DMSO, for a total of 11 test concentrations. A 4 μl aliquot of each dilution was added to 196 of broth medium seeded with the organism suspension in wells of a 96 well plate (bacterial count: 2 −8×105 colony forming units/mL final). The final volume was 200 μl in each well and the final DMSO concentration was 2 percent. Test concentrations were 128 to 0.125 μg/mL.

The medium, the incubation time and temperature are listed in the Table 7.

TABLE 7

| | | Broth | Aerobic/ | Incubation |
|---|---|---|---|---|
| Strain | Organism | medium | Anaerobic | time |
| ATCC 12386 | Streptococcus | CAMHB II, | Aerobic | 1 day at |
| | agalactiae | 5% LHB | | 36° C. |
| ATCC 19615 | Streptococcus | CAMHB II, | Aerobic | 1 day at |
| | pyogenes | 5% LHB | | 36° C. |
| ATCC BAA-1717 | Staphylococcus | CAMHB II | Aerobic | 1 day at |
| | aureus | | | 36° C. |
| ATCC BAA-1556 | Staphylococcus | CAMHB II | Aerobic | 1 day at |
| | aureus | | | 36° C. |
| ATCC 29213 | Staphylococcus | CAMHB II | Aerobic | 1 day at |
| | aureus | | | 36° C. |
| ECL 2963646 | Staphylococcus | CAMHB II | Aerobic | 1 day at |
| | aureus | | | 36° C. |
| NARSA NRS119 | Staphylococcus | CAMHB II | Aerobic | 1 day at |
| | aureus | | | 36° C. |
| NARSA NRS127 | Staphylococcus | CAMHB II | Aerobic | 1 day at |
| | aureus | | | 36° C. |
| NARSA NRS22 | Staphylococcus | CAMHB II | Aerobic | 1 day at |
| | aureus | | | 36° C. |
| NARSA NRS382 | Staphylococcus | CAMHB II | Aerobic | 1 day at |
| | aureus | | | 36° C. |
| NARSA NRS383 | Staphylococcus | CAMHB II | Aerobic | 1 day at |
| | aureus | | | 36° C. |
| NARSA VRS2 | Staphylococcus | CAMHB II | Aerobic | 1 day at |
| | aureus | | | 36° C. |
| ATCC 12228 | Staphylococcus | CAMHB II | Aerobic | 1 day at |
| | epidermidis | | | 36° C. |
| ATCC 33591 | Staphylococcus | CAMHB II | Aerobic | 1 day at |
| | aureus | | | 36° C. |
| ATCC BAA-1805 | Clostridium | RCM | Anaerobic | 2 days at |
| | difficile | | | 36° C. |

* CAMHB II = Cation-adjusted Mueller-Hinton Broth II; CAMHB II, 5% LHB = Cation-adj. Muller-Hinton Broth with 5% lysed horse blood; RCM = Reinforced Clostridial Medium.

Following incubation, the plates were visually examined, and wells were scored for growth or complete growth inhibition to define the minimum inhibitory concentration. Each test substance was evaluated in duplicate and the results below are the duplicate test values. Vehicle-control and Vancomycin or Linezolid, as depicted at Table 8, were used as blank and positive controls, respectively.

TABLE 8

| | | | MIC | |
|---|---|---|---|---|
| | MIC of | Reference | (Concurrent) | |
| Strain | 6027 | compound | μg/mL | Resistance |
| ATCC 12386 | 1 | Vancomycin | 0.5 | — |
| ATCC 19615 | 4 | Vancomycin | 0.25 | — |
| ATCC BAA-1717 | 1 | Vancomycin | 0.5 | Methicillin resistant |
| ATCC BAA-1556 | 2 | Linezolid | 2 | Methicillin resistant (Oxacillin |
| | | | | resistance confirmed) |
| ATCC 29213 | 1 | Vancomycin | 0.5 | — |
| ECL 2963646 | 2 | Linezolid | 2 | Gentamicin, Levofloxacin, |
| | | | | Vancomycin, Clindamycin, |
| | | | | Erythromycin resistant |
| NARSA NRS119 | 2 | Vancomycin | 1 | Linezolid-resistant, Methicillin |
| | | | | resistant |
| NARSA NRS127 | 2 | Vancomycin | 1 | Linezolid-resistant, Methicillin |
| | | | | resistant |
| NARSA NRS22 | 2 | Linezolid | 2 | Methicillin resistant |
| NARSA NRS382 | 1 | Vancomycin | 1 | Methicillin resistant |
| NARSA NRS383 | 2 | Vancomycin | 0.5 | Methicillin resistant |
| NARSA VRS2 | 2 | Linezolid | 2 | — |
| ATCC 12228 | 2 | Vancomycin | 1 | — |
| ATCC 33591 | 4 | Vancomycin | 1 | Methicillin resistant |
| ATCC BAA-1805 | 0.25 | Vancomycin | 0.5 | — |

The antibacterial potency of inventive compounds was measured using the in vitro broth microdilution assay under assay conditions described by the Clinical and Laboratory Standards Institute. In this assay, the Minimum Inhibitory Concentration (MIC) is defined as the lowest concentration of an agent that completely inhibits visible growth in vitro of the microorganism. Pre-formulated compound solution was diluted in 100% DMSO to prepare a 1.6 mg/mL working stock solution. The 1.6 mg/mL working stock solution was diluted by 2-fold serial titrations in 100% DMSO, for a total of 11 test concentrations. A 4 μL aliquot of each dilution was added to 196 μL of broth medium seeded with the organism suspension in wells of a 96 well plate (bacterial cell count: 2−5×105 colony forming units/mL final). The final volume was 200 μL in each well and the final DMSO concentration was 2 percent. The concentration range was 0.032 to 32 μg/mL. The medium, the incubation time and temperature are listed in the Table 7. Following incubation, the test plates were visually examined, and wells were scored for growth or complete growth inhibition to define the minimum inhibitory concentration (MIC). Vehicle-control and Vancomycin were used as blank and positive controls, respectively.

TABLE 9
Antibacterial potency of inventive compounds against
Staphylococcus aureus ATCC BAA-1556, USA300 MRSA

| Compound | MIC (μg/mL) |
|---|---|
| Vancomycin | 1 |
| 6025 | 4 |
| 6027 | 1 |
| 6483 | 8 |
| 6789 | 16 |
| 6791 | 32 |
| 7058 | 4 |
| 7059 | 4 |
| 7192 | 16 |

### Example 8

### In Vitro Antibacterial Assay Method 2

The antibacterial activity of inventive compounds was evaluated in duplicate in a further Minimum Inhibitory Concentration (MIC) assay.

Bacteria were cultured in Cation-adjusted Mueller Hinton broth (CAMHB) at 37° C. overnight. A sample of each culture was then diluted 40-fold in fresh broth and incubated at 37° C. for 1.5-3 h. The resultant mid-log phase cultures were diluted (CFU/mL measured by OD600), then added to each well of the compound containing plates, giving a cell density of 5×10⁵ CFU/mL and a total volume of 50 μL. All the plates were covered and incubated at 37° C. for 18 h without shaking.

Samples were prepared in DMSO and serially diluted 2 fold 8 times. Each sample concentration was prepared in 384-well plates, non-binding surface plate (NBS; Corning 3640) for the bacterial strain ATCC 43300, MRSA. The final DMSO concentration was at a maximum of 0.5%.

Inhibition of bacterial growth was determined measuring absorbance at 600 nm (OD600), using a Tecan M1000 Pro monochromator plate reader. The percentage of growth inhibition was calculated for each well, using the negative control (media only) and positive control (bacteria without inhibitors) on the same plate as references. The MIC was determined as the lowest concentration at which the growth was fully inhibited, defined by an inhibition >80%. Vancomycin was used as positive bacterial inhibitor standards.

TABLE 10
Antibacterial potency of inventive compounds against
Staphylococcus aureus ATCC 43300, MRSA

| Compound | MIC (μg/mL) |
|---|---|
| 6027 | 2 |
| 6483 | 8 |
| 6779 | 8 |
| 7059 | 32 |
| 7192 | 8 |
| 7846 | 4 |
| 7848 | 4 |
| 7842 | 32 |

### Example 9

### In Vivo Antibacterial Assay

### S. aureus (USA300 MRSA, BAA-1556) Dermal Infection Model, CFU/Skin

Inoculum preparation. The testing strain, S. aureus USA300 MRSA (BAA-1556), was obtained from the frozen working stock and thawed at room temperature. A 0.2 mL aliquot was inoculated into 20 mL Brain-Heart Infusion broth (BHI) and then incubated at 35-37° C. with shaking (120 rpm) for 8 h. Bacterial cells in the 20 mL culture were pelleted by centrifugation at 3,500×g for 15 minutes and then re-suspended in 10 mL cold PBS (>8.0×109 CFU/mL, OD620 1.8-2.0). The culture was then diluted in PBS to the target inoculum of 1.0×107 CFU/mL. The actual bacterial counts were determined by plating dilutions onto nutrient agar (NA) plates followed by 20-24 h incubation and colony counting. The actual CFU counts were 1.04×107 CFU/mL.

S. aureus (USA300 MRSA, BAA-1556) dermal infection model. Groups of 5 female ICR mice weighing 24±2 g were used. Animals were anesthetized with etomidate-lipuro emulsion (20 mg/10 mL) at 20 mg/kg by intravenous (IV) injection, and then the fur on the back was removed by an electric shaver, and the epidermal layer was disrupted with an abrasive paper. Mice were inoculated topically (TOP) on the wound area with S. aureus (MRSA, BAA-1556) suspension, 5 μL/mouse. The target inoculation density was 5×104 CFU/mouse and the actual inoculum count 5.2×104 CFU/mouse. Animals were housed separately after infection. Compound 6027 in DMSO at 1% was administered topically (TOP) twice (BID) at 1 and 7 h post infection. Reference control agent, fusidic acid cream at 20 mg/gram (2%), was administered TOP BID at 1 and 7 h post infection. One infected and untreated group was sacrificed at 1 h after infection for the initial (baseline) bacterial counts. The dose volumes were 20 μL/mouse for all dosing groups.

Animals were sacrificed at 1 or 25 h after inoculation with CO₂ asphyxiation. The infected skin samples of the wound infection (around 2 cm 2 areas) were excised and were homogenized in 1 mL PBS (pH 7.4) with a polytron homogenizer. A 0.1 mL aliquot of each homogenate was used for serial 10-fold dilutions and plated onto NA plates for bacterial enumeration. The bacterial counts (CFU/skin) in skin tissues were calculated and the percentage decrease in counts compared to the corresponding vehicle control (placebo) was calculated with the following formula: Decrease (%)=[(CFU/skin of placebo —CFU/skin of treatment)/(CFU/skin of placebo)]×100%.

Results. Twenty-five hours post inoculation the group treated with 1% 6027 in DMSO showed a 71% reduction in colony forming units (CFUs) over the DMSO treated DMSO control group. The positive control fusidic acid (2%) showed 99% reduction in CFUs over control.

TABLE 11
In vivo reduction of Staphylloccocus areus (USA300 MRSA, BAA-1556)
colony forming units (CFU) of 6027 (1% in DMSO) and positive
control fusidic acid (2%) after 25 hours post inoculation, and
treatment twice a day (hour 1 and 7 post inoculation). Results
shown in Log CFU with standard error of the mean (SEM).

| | | LogCFU/skin after |
|---|---|---|
| | Decrease | 25 hours (Standard error |
| Sample | (%) | of the mean − SEM) |
| 6027 (1% in DMSO) | 71 | 6.50 (0.16) |
| DMSO | 0 | 7.09 (0.13) |
| Fusidic acid 2% | 99 | 4.82 (0.26) |

### Example 10

### In Vitro Antifungal Activity

The antifungal potency of the very preferred compound of the present invention 6027 was measured using the in vitro broth microdilution assay. In this assay, the Minimum Inhibitory Concentration (MIC) is defined as the lowest concentration of an agent that completely inhibits visible growth in vitro of the microorganism. The assay conditions, described by the Clinical and Laboratory Standards Institute were used for preparation of the inoculum, growth medium, and end point reading. Pre-formulated compound solution was diluted in 100% DMSO to prepare a 1.6 mg/mL working stock solution. The 1.6 mg/mL working stock solution was diluted by 2-fold serial titrations in 100% DMSO, for a total of 11 test concentrations. A 4 μl aliquot of each dilution was added to 196 μl of broth medium seeded with the organism suspension in wells of a 96 well plate (fungal cell count: 1×103 to 1×104 final). The final volume was 200 μl in each well and the final DMSO concentration was 2 percent. Test concentration range was 0.032 to 32 μg/mL. The medium, the incubation time and temperature are listed in the Table 12.

TABLE 12
Tested organisms and test conditions
for the antifungal potency assay.

| | | | Aerobic/ | Incubation |
|---|---|---|---|---|
| Strain | Organism | Broth medium | Anaerobic | time |
| ATCC | Trichophyton | RPMI-1640, | Aerobic | 2 d at 28° C. |
| 9533 | mentagrophytes | MOPS | | |
| ATCC | Trichophyton | RPMI-1640, | Aerobic | 7 d at 28° C. |
| 10218 | rubrum | MOPS | | |

* RPMI-1640, MOPS = RPMI-1640 with Glutamine, Phenol Red, and 165 mM MOPS pH 7.0; RPMI-1640, MOPS = RPMI-1640 with Glutamine, Phenol Red, and 165 mM MOPS pH 7.0.

Following incubation, the plates were visually examined, and wells were scored for growth or complete growth inhibition to define the minimum inhibitory concentration. Vehicle-control and an active reference agent were used as blank and positive controls.

TABLE 13
Antifungal potency of inventive compounds against
Trichophyton mentagrophytes (ATCC 9533) &
Trichophyton rubrum (ATCC 10218)

| | T. mentagrophytes | T. rubrum |
|---|---|---|
| | (ATCC 9533) | (ATCC 10218) |
| Compound | MIC (μg/mL) | MIC (μg/mL) |
| 6027 | 16 | 32 |

1. A compound of formula (I) for use in a method of topical treatment of a skin disease or skin disorder of a mammal, wherein said method comprises topical administration of said compound to said mammal, 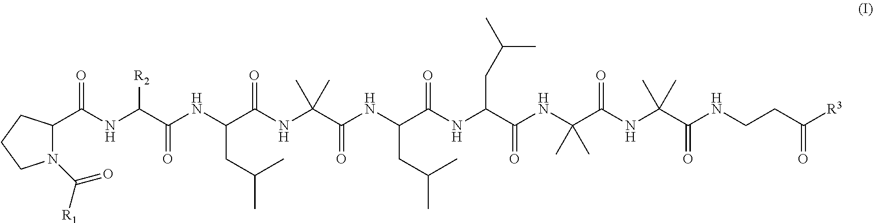
wherein
R₁ is selected from carbocyclyl or heterocyclyl, each independently optionally substituted with C₁-C₄alkyl, halogen, oxo, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl;
R₂ is selected from C₅-C₁₂alkyl, C₄-C₁₀alkoxy, C₁-C₃alkylene-cycloalkyl, C₁-C₃alkylene-aryl, C₁-C₃alkylene-heteroaryl, wherein said alkyl, cycloalkyl, aryl and heteroaryl are each independently optionally substituted with one or more, typically and preferably one or two, substituents selected from C₁-C₂alkyl, C₁-C₂haloalkyl, halogen, C₁-C₂alkoxy;
R₃ is 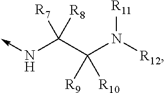
wherein
R₇, R₈, R₉ and R₁₀ are independently at each occurrence H or C₁-C₃alkyl, preferably H or methyl, or independently at each occurrence two of said R₇, R₈, R₉ and R₁₀ together with the carbon atom to which they are attached form a carbocyclic or heterocyclic ring, preferably a carbocyclic ring, and wherein
R₁₁ and R₁₂ are independently of each other H or C₁-C₄alkyl optionally substituted with halogen, hydroxyl or C₃-C₆cycloalkyl; or together with the nitrogen atom to which they are attached form independently at each occurrence a heteroaryl or a heterocyclyl, each independently optionally substituted with halogen, C₁-C₄alkyl, OR₁₃, NR₁₄R₁₅; wherein R₁₃, R₁₄, R₁₅ are independently at each occurrence H, C₁-C₄alkyl, and wherein the arrow indicates the attachment to the C(O)-moiety depicted in formula (I);
and pharmaceutically acceptable salts of said compound of formula (I).

2. The compound for use of claim 1, wherein said compound of formula (I) is a compound of any one of the formula (II) to (IV) 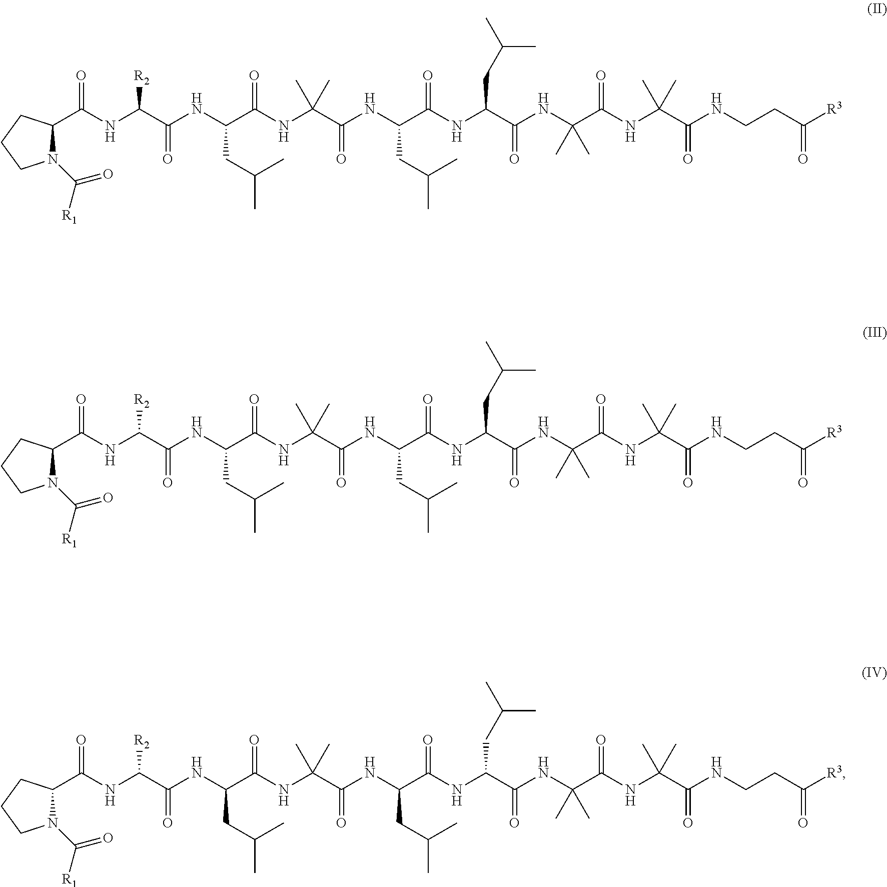
wherein preferably said compound of formula (I) is a compound of formula (II).

3. The compound for use of claim 1 or claim 2, wherein said R₁ is selected from cycloalkyl, aryl or heteroaryl, preferably selected from cycloalkyl, monocyclic or bicyclic aryl or monocyclic or bicyclic heteroaryl, each independently optionally substituted with C₁-C₄alkyl, halogen, oxo, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl.

4. The compound for use of any one of the claims 1 to 3, wherein said R₁ is selected from phenyl or a monocyclic heteroaryl comprising one or two heteroatoms selected from N, O and S, preferably from phenyl or oxazolyl; each independently optionally substituted with C₁-C₄alkyl, halogen, CF₃, OR₄, NR₅R₆, C₆H₅, C₆H₅ substituted with halogen, C₁-C₃alkyl, OR₄, NR₅R₆, wherein R₄, R₅, R₆ are independently at each occurrence H, C₁-C₃alkyl.

5. The compound for use of any one of the claims 1 to 4, wherein said R₁ is selected from the formula 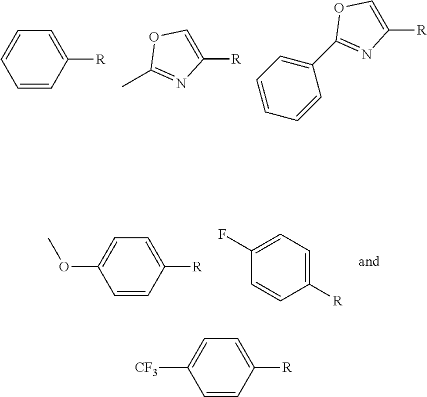
wherein R indicates the attachment to the C(O)-moiety depicted in formula (I).

6. The compound for use of any one of the claims 1 to 5, wherein said R₂ is selected from C₅-C₁₂alkyl, C₄-C₁₀alkoxy, C₁-C₃alkylene-C₅-C₆cycloalkyl, C₁-C₃alkylene-phenyl, C₁-C₃alkylene-(mono- or bicyclic-heteroaryl), wherein said phenyl, C₅-C₆-cycloalkyl, and mono- or bicyclic-heteroaryl are each independently optionally substituted with one or more, typically and preferably with one or two, substituents selected from C₁-C₂alkyl, C₁-C₂haloalkyl, halogen, C₁-C₂alkoxy.

7. The compound for use of any one of the claims 1 to 6, wherein said R₂ is selected from C₅-C₁₂alkyl, C₁-C₂alkylene-C₅-C₆cycloalkyl and CH₂-phenyl, wherein said phenyl is optionally substituted with one or two substituents selected from methyl, ethyl, fluorine, chlorine and methoxy.

8. The compound for use of any one of the claims 1 to 7, wherein said R₂ is selected from 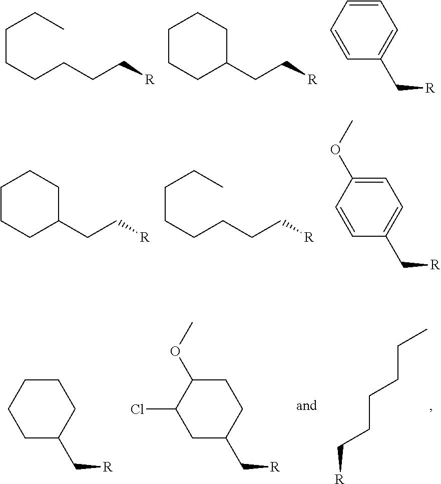
wherein R indicates the attachment to the CH-moiety depicted in formula (I).

9. The compound for use of any one of the claims 1 to 8, wherein said R₇, R₈, R₉ and R₁₀ are independently at each occurrence H or C₁-C₃alkyl, preferably H or methyl, or independently at each occurrence two of said R₇, R₈, R₉ and R₁₀ together with the carbon atom to which they are attached form a monocyclic carbocyclic or monocyclic heterocyclic ring, preferably a monocyclic carbocyclic ring, and wherein R₁₁ and R₁₂ are independently of each other H or C₁-C₄alkyl optionally substituted with halogen, hydroxyl or C₃-C₆cycloalkyl; or together with the nitrogen atom to which they are attached form independently at each occurrence a monocyclic heteroaryl or a monocyclic heterocyclyl, each independently optionally substituted with halogen, C₁-C₄alkyl, OR₁₃, NR₁₄R₁₅; wherein R₁₃, R₁₄, R₁₅ are independently at each occurrence H, C₁-C₄alkyl.

10. The compound for use of any one of the claims 1 to 9, wherein said R₃ is selected from 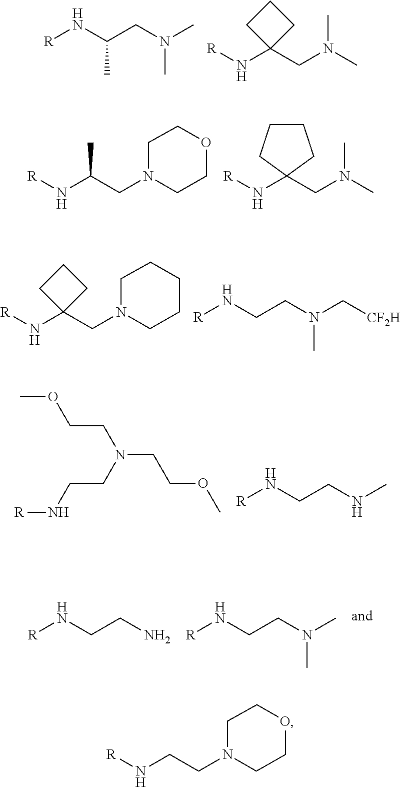
wherein R indicates the attachment to the C(O)-moiety depicted in formula (I).

11. The compound for use according to claim 1, wherein said compound is selected from

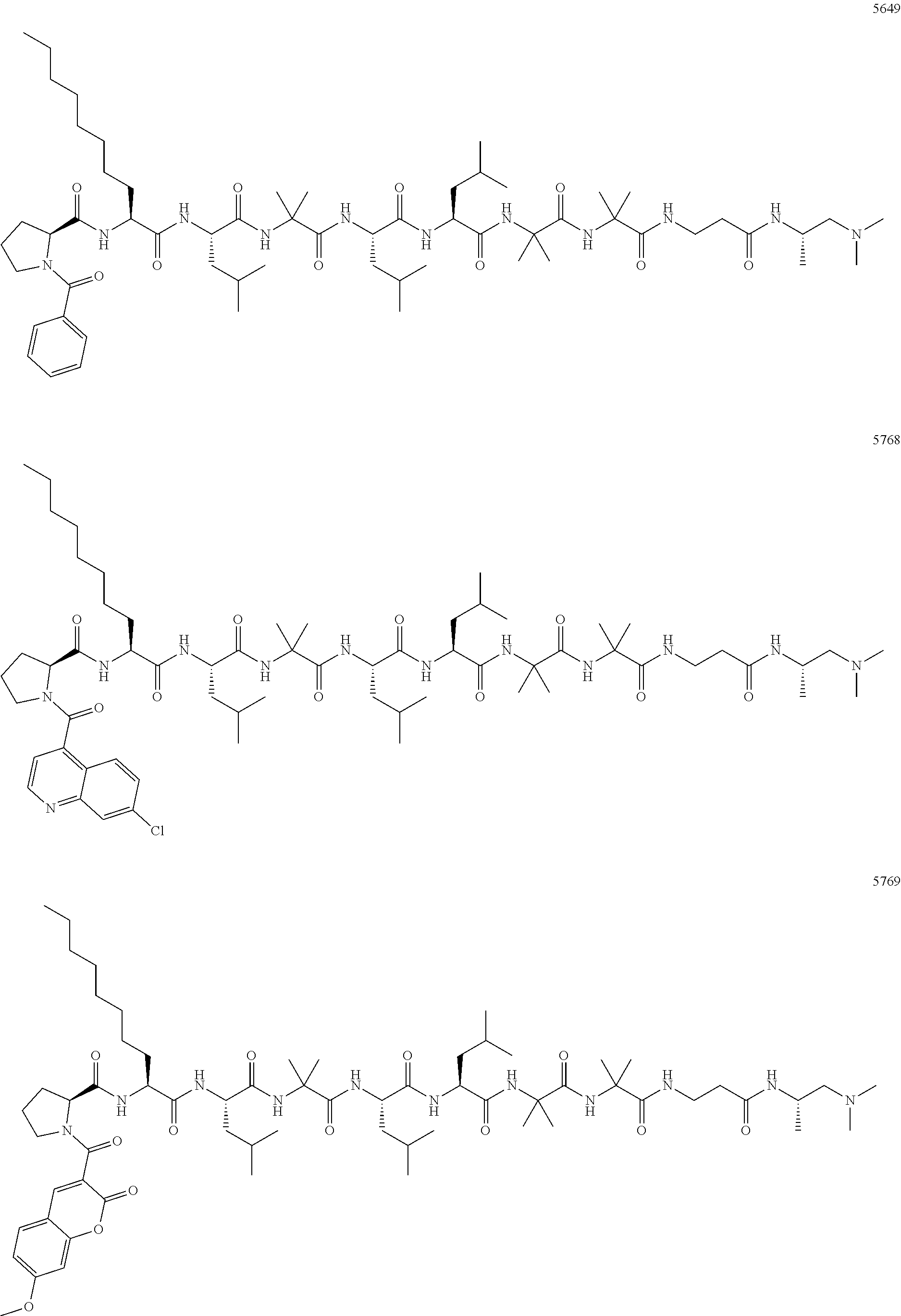

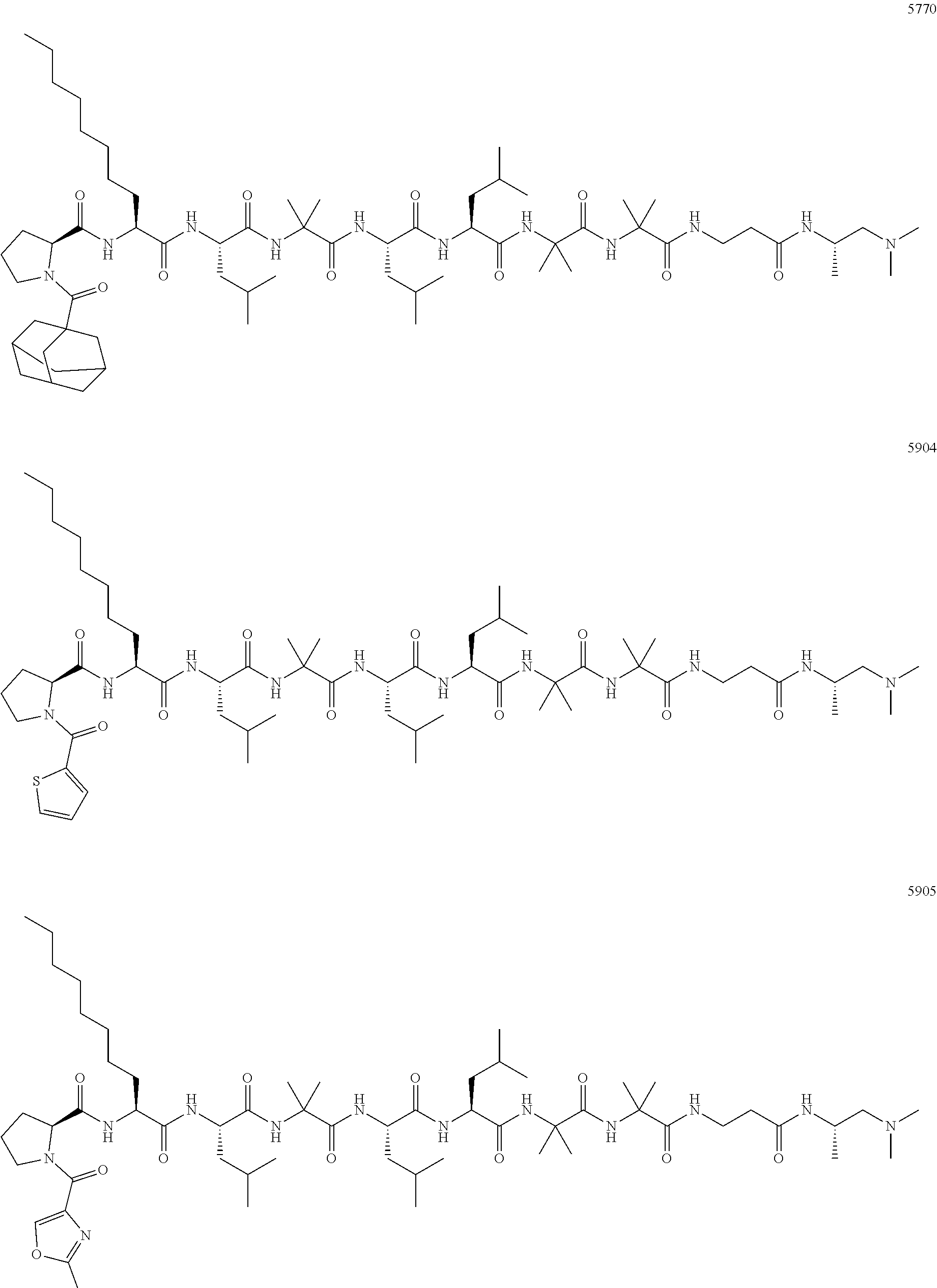

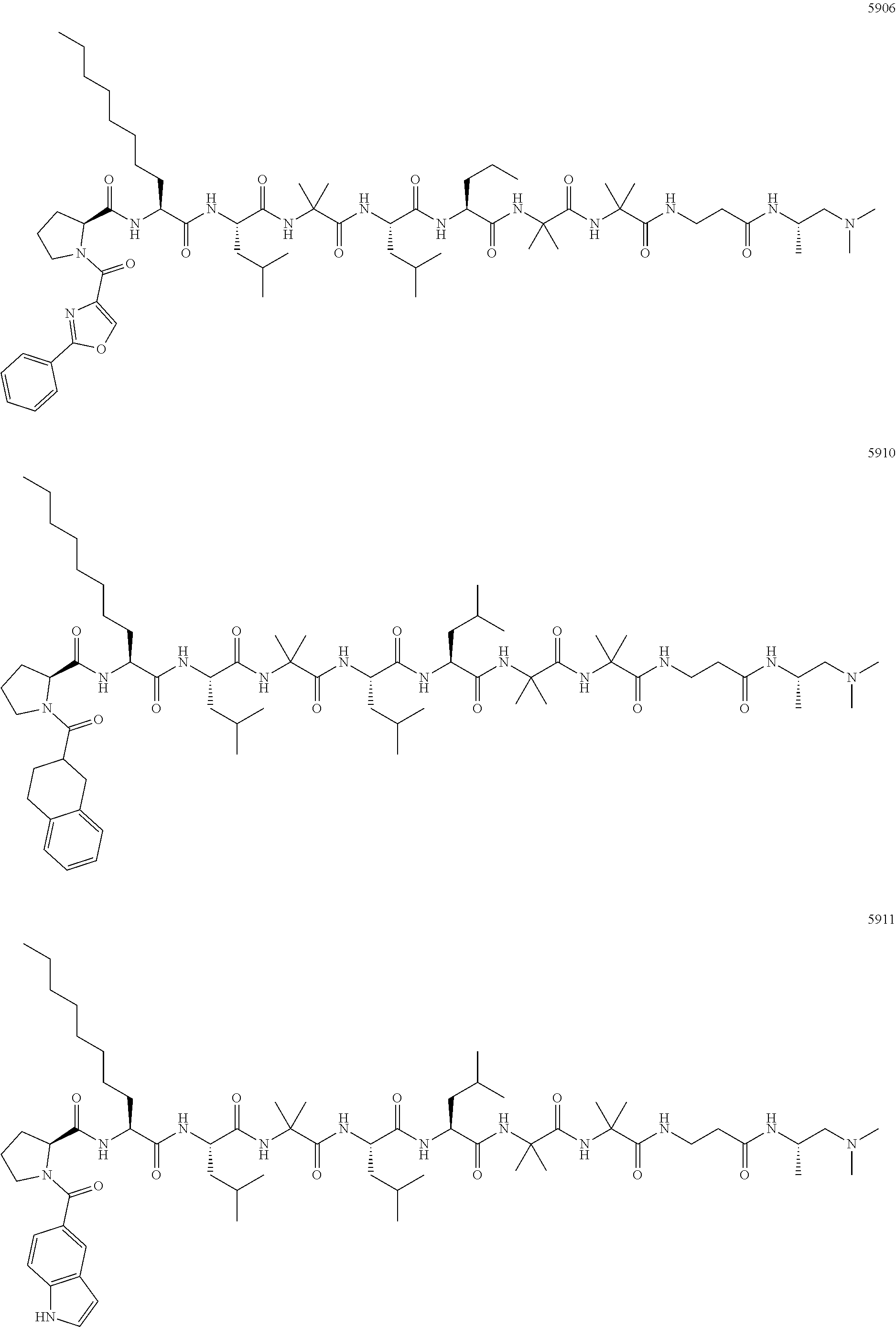

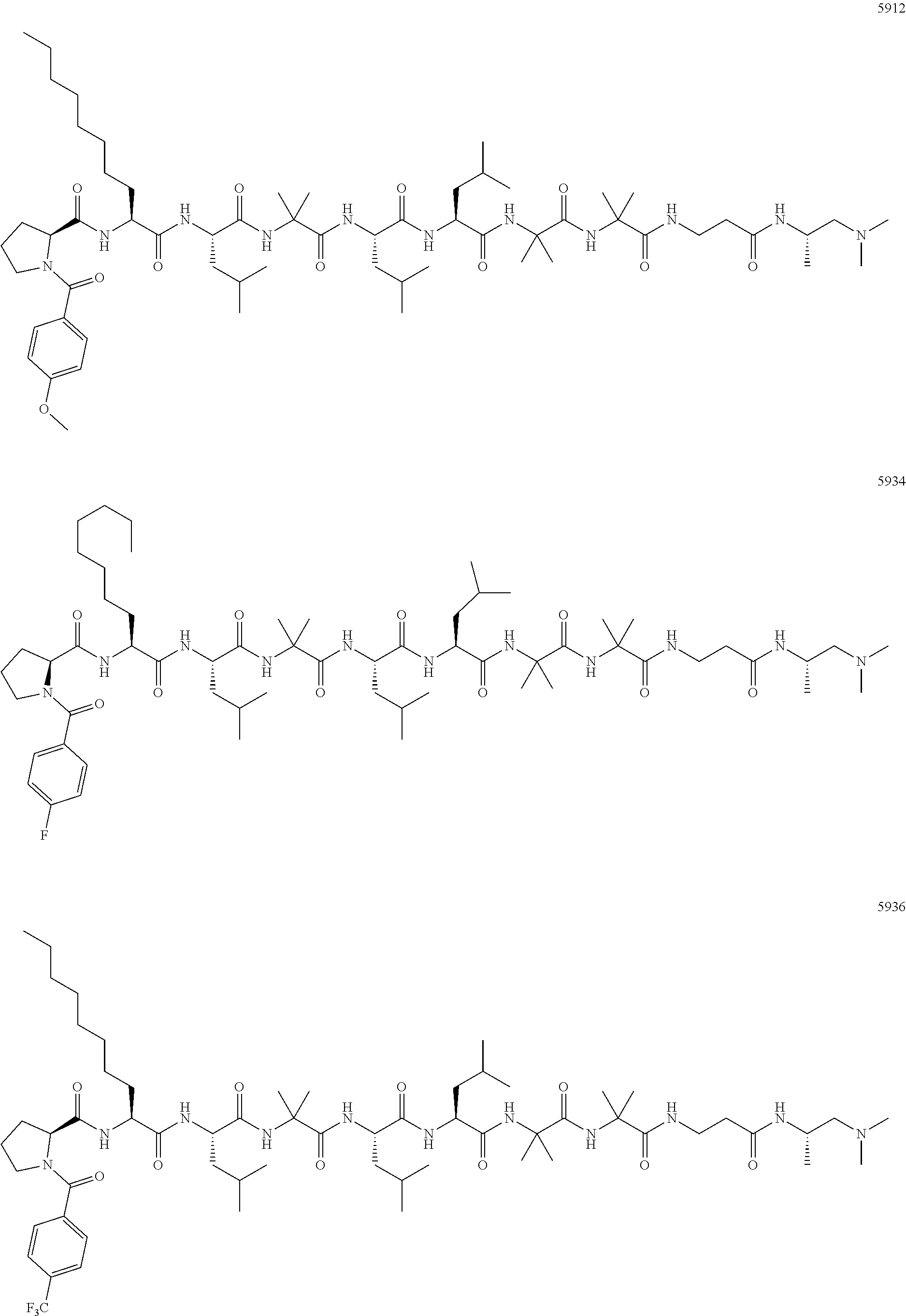

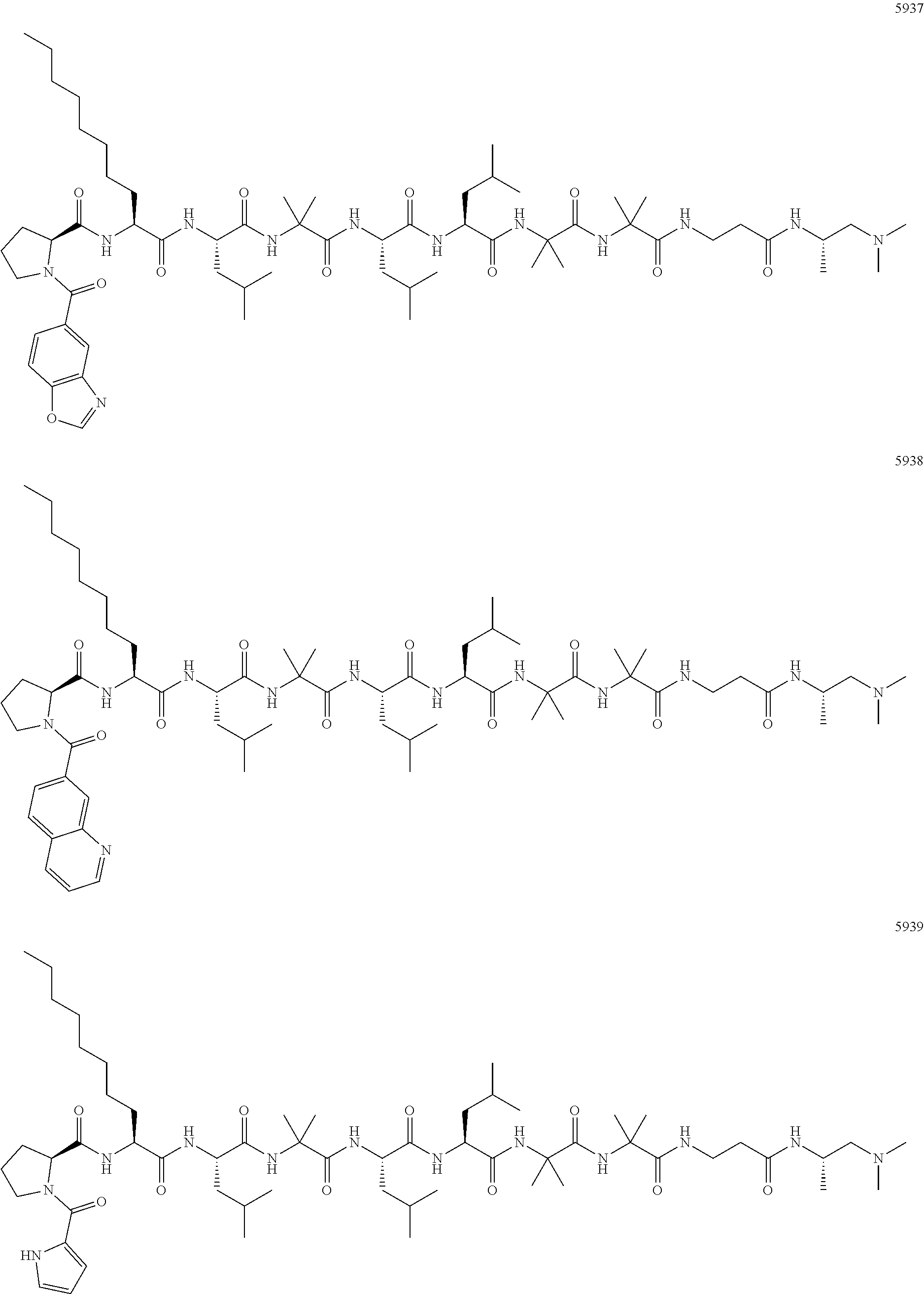

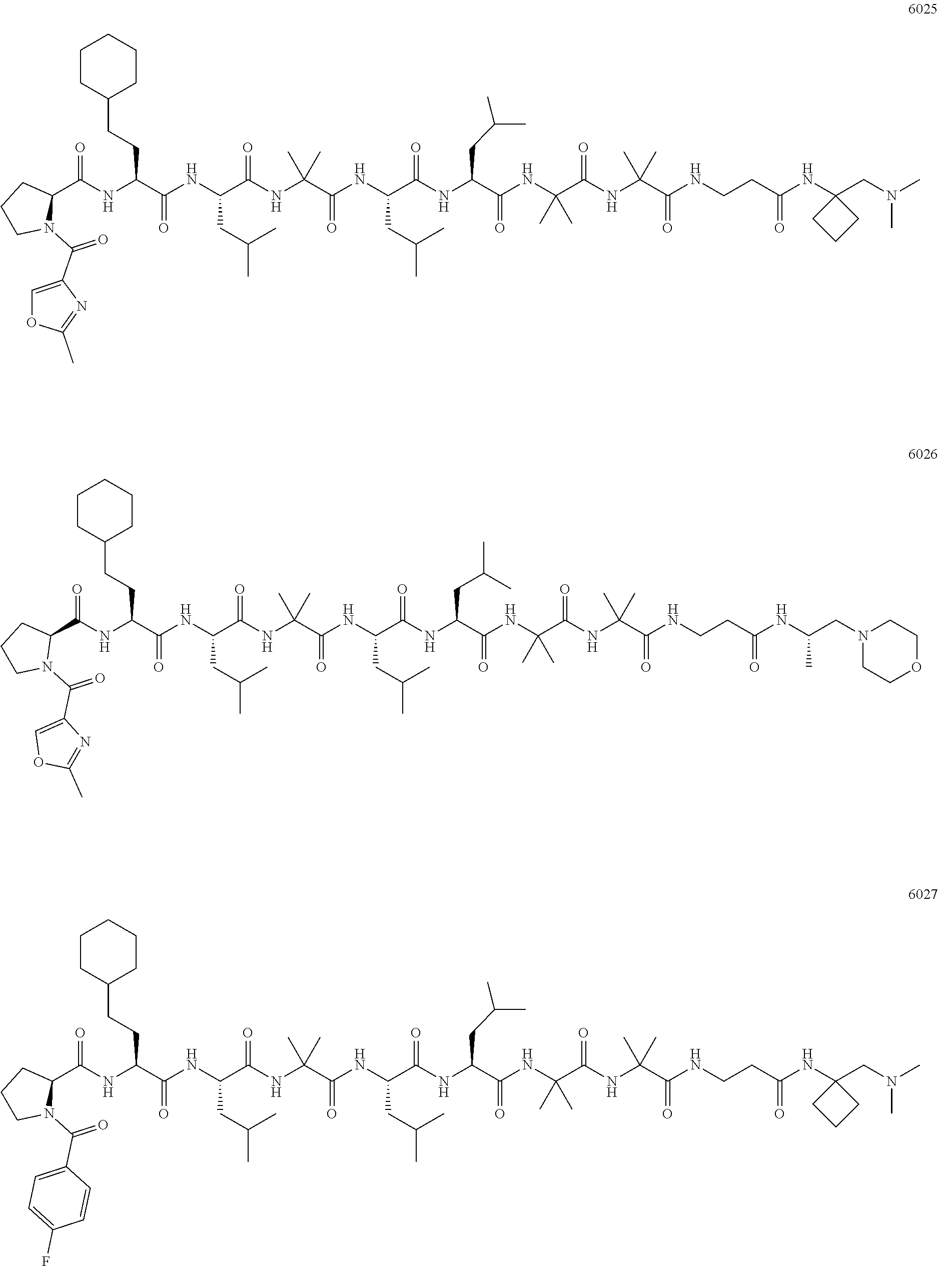

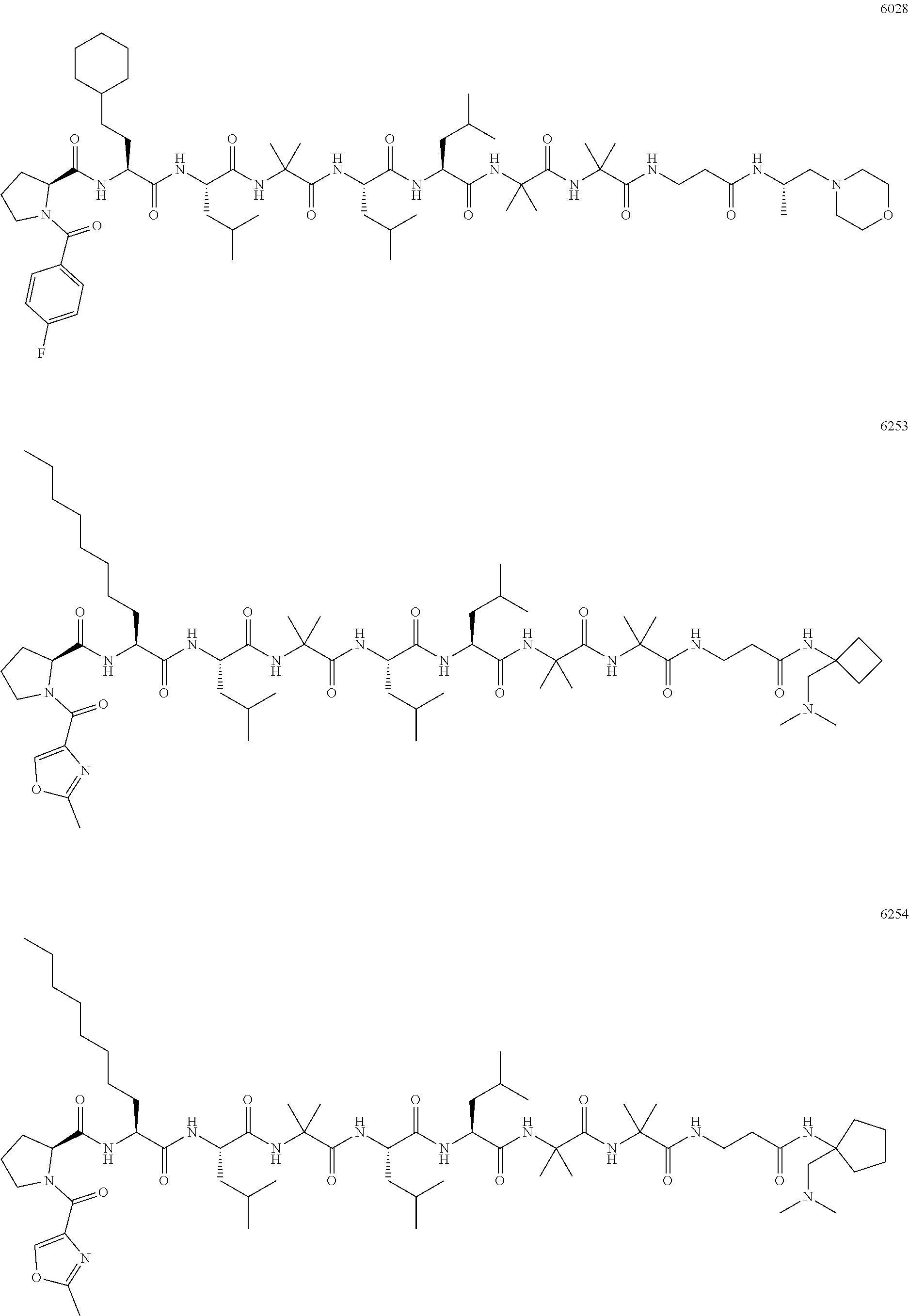

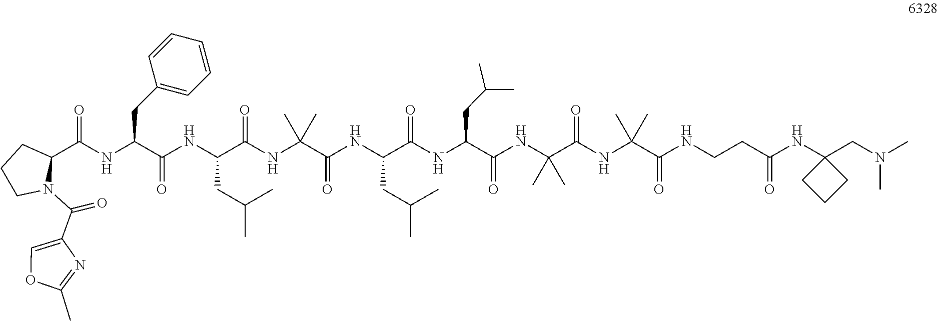 [SMILES: Cc1nc(C(=O)N2CCC[C@H]2C(=O)N[C@@H](Cc2ccccc2)C(=O)N[C@@H](CC(C)C)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NC2(CN(C)C)CCC2)co1]

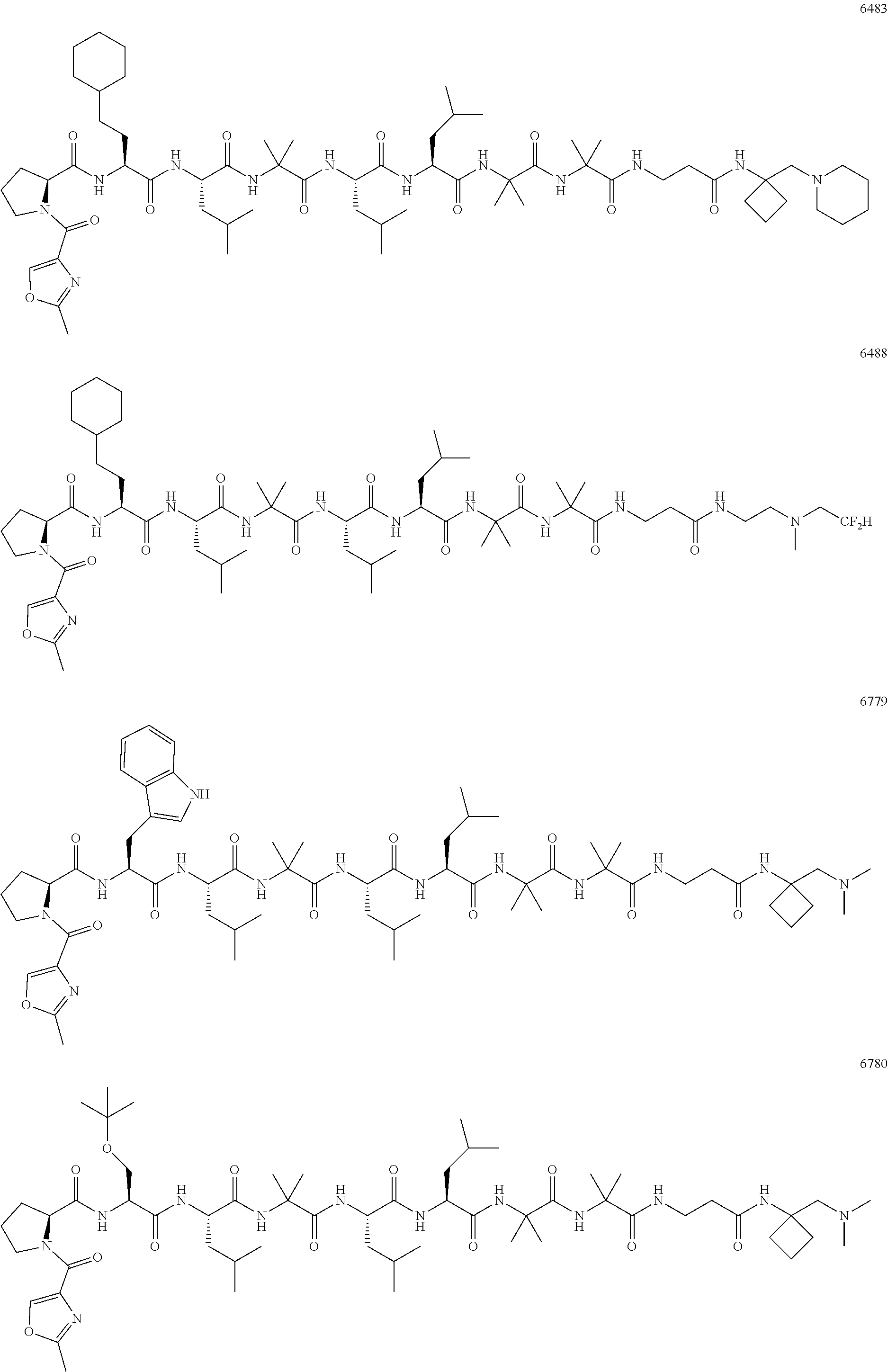

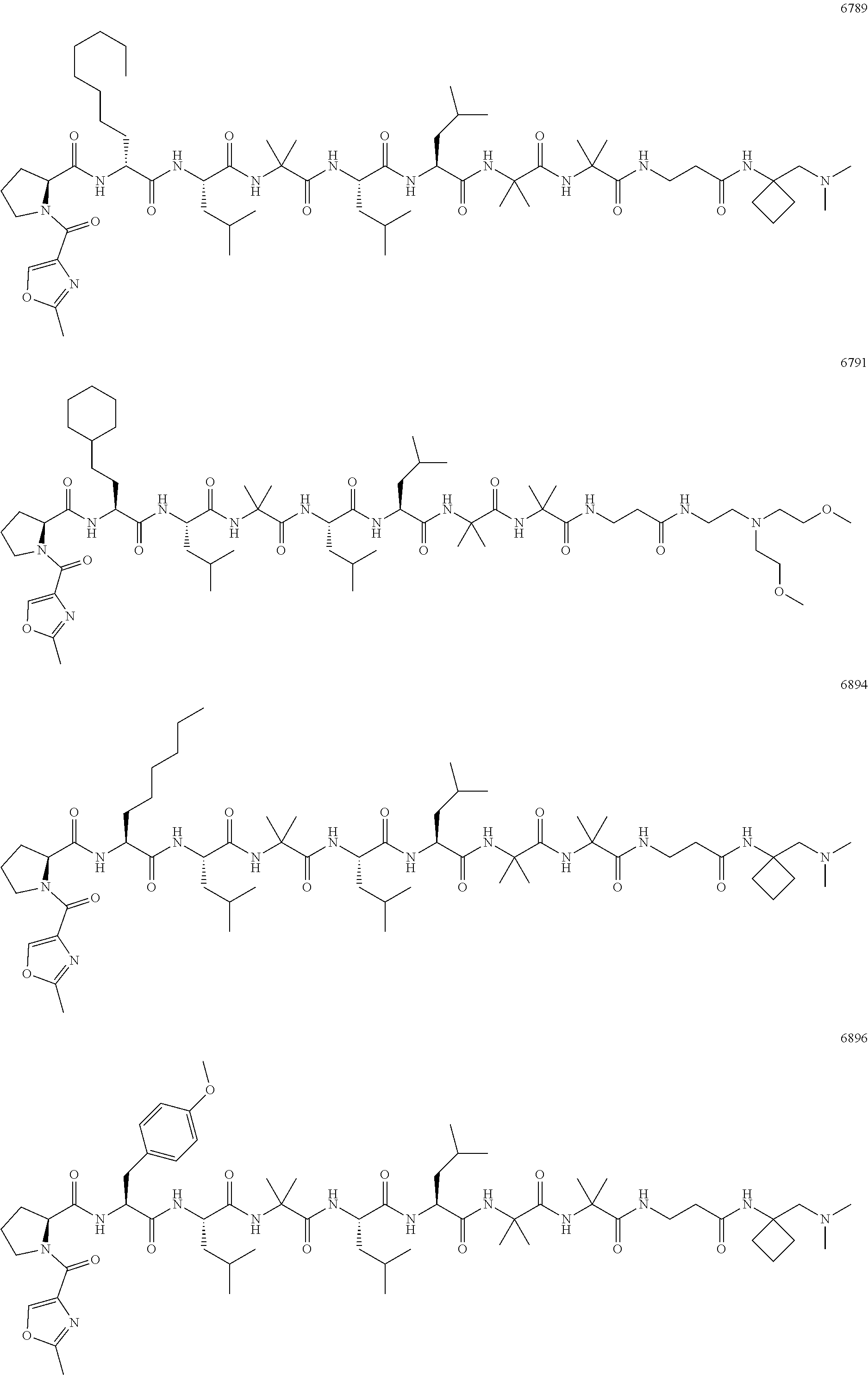

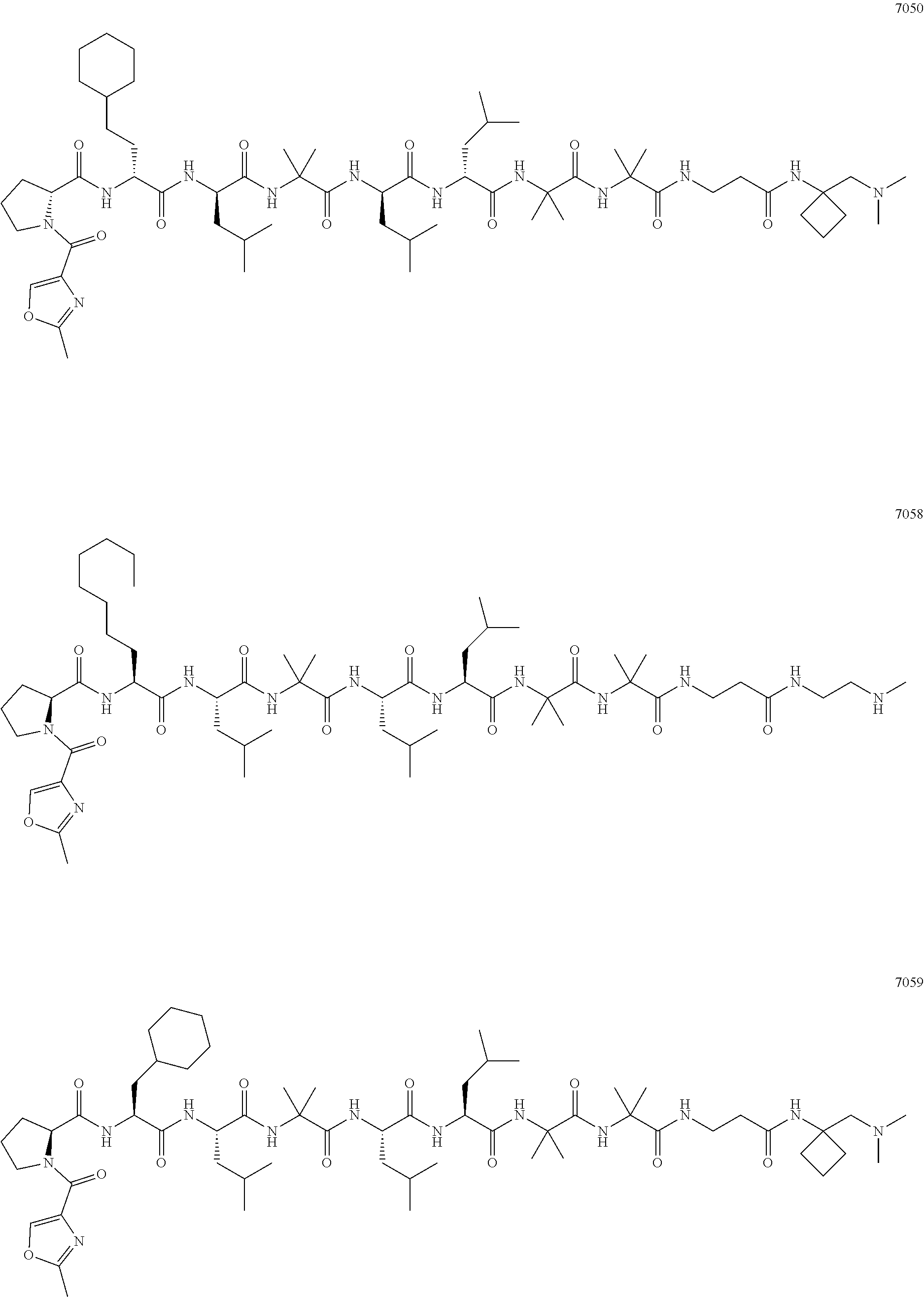

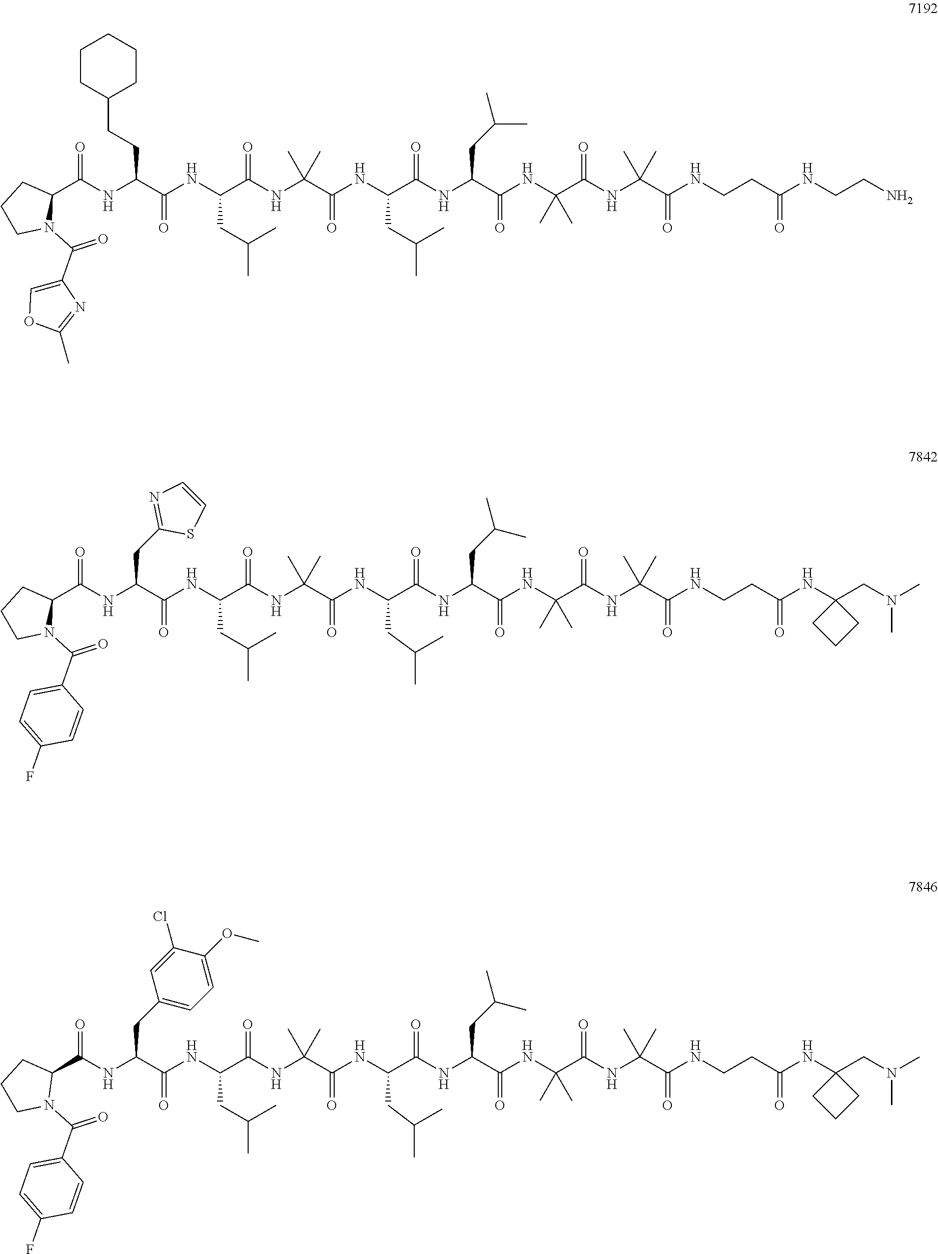

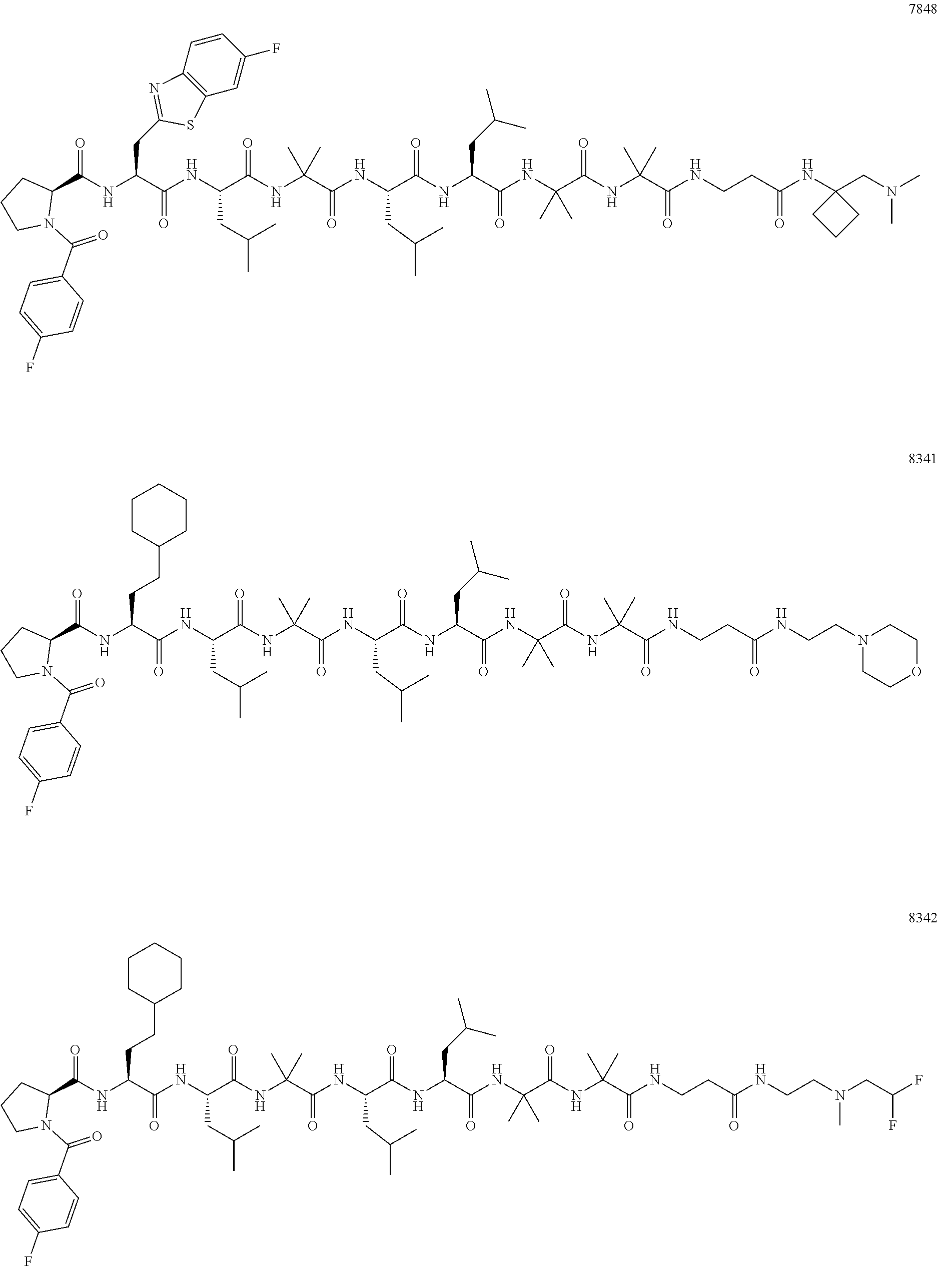

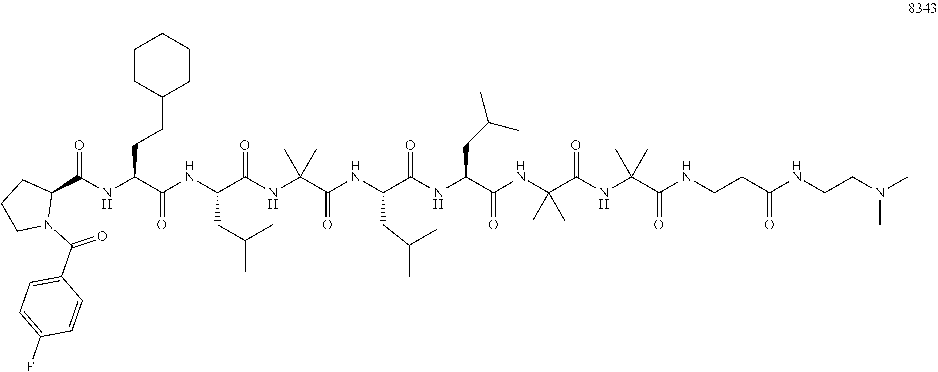 [SMILES: CC(C)C[C@H](NC(=O)[C@H](CCC1CCCCC1)NC(=O)[C@@H]1CCCN1C(=O)c1ccc(F)cc1)C(=O)NC(C)(C)C(=O)N[C@@H](CC(C)C)C(=O)N[C@@H](CC(C)C)C(=O)NC(C)(C)C(=O)NC(C)(C)C(=O)NCCC(=O)NCCN(C)C]

12. The compound for use of any one of the claims 1 to 11, wherein said topical administration is applying said compound to a skin of a mammal, preferably to a skin of a human.

13. The compound for use of any one of the claims 1 to 12, wherein said skin disease or skin disorder is selected from a protozoan disease, a bacterial infection or a fungal infection.

14. The compound for use of any one of the claims 1 to 12, wherein said skin disease or skin disorder is cutaneous leishmaniasis.

15. A pharmaceutical composition for use in a method of topical treatment of a skin disease or skin disorder of a mammal, wherein said pharmaceutical composition comprises a compound of formula (I) or a pharmaceutically acceptable salt thereof as defined in any one of the claims 1 to 14 and a pharmaceutically acceptable carrier or adjuvant, and wherein said method comprises topical administration of said pharmaceutical composition to said mammal.
